(19) United States
(12) Patent Application Publication
(10) Pub. No.: US 2026/0308262 A1
(43) Pub. Date: Oct. 8, 2026

(54) ANTI-CD161 ANTIBODIES AND METHODS OF USE THEREOF

(71) Applicant: NOVASENTA, INC., Pittsburgh, PA (US)

(72) Inventors: Jiayi YU, Pittsburgh, PA (US); Sanjay RATHOD, Pittsburgh, PA (US); Krishnadatt PERSAUD, Pittsburgh, PA (US); Mani MOHINDRU, Pittsburgh, PA (US); Pallavi TAWDE, Pittsburgh, PA (US)

(73) Assignee: NOVASENTA, INC., Pittsburgh, PA (US)

(21) Appl. No.: 19/481,438

(22) PCT Filed: May 6, 2024
(86) PCT No.: PCT/US2024/028001

§ 371 Date: Nov. 4, 2025

Related U.S. Application Data
(60) Provisional application No. 63/500,122, filed on May 4, 2023.

(51) Int. Cl. C07K 16/28; A61K 39/00; A61P 35/00

(52) U.S. Cl. CPC C07K 16/2851; A61P 35/00; A61K 2039/505

(57) ABSTRACT

The present disclosure provides antibodies and antigen-binding portions thereof that bind CD161, bispecific antibodies and multispecific antibodies comprising the same, nucleic acids and vectors encoding the same, and methods of using the same to treat a subject in need thereof.

### CROSS-REFERENCE TO RELATED APPLICATIONS

This PCT application claims the priority benefit of U.S. Provisional Application No. 63/500,122, filed May 4, 2023, which is herein incorporated by reference in its entirety.

### REFERENCE TO SEQUENCE LISTING SUBMITTED ELECTRONICALLY

The content of the sequence listing is submitted electronically (Name: 5037_001PC01_Seqlisting_ST26; Size: 304,752 bytes; and Date of Creation: May 3, 2024) with the application and herein incorporated by reference in its entirety.

### FIELD

The present disclosure relates to antibodies and antigen-binding portions thereof that bind CD161 and methods of using the same to treat a subject in need thereof.

### BACKGROUND

Human cancers harbor numerous genetic and epigenetic alterations, generating neoantigens potentially recognizable by the immune system (Sjoblom et al., Science 2006) 314 (5797): 268-274). The adaptive immune system, comprised of T and B lymphocytes, has powerful anti-cancer potential, with a broad capacity and exquisite specificity to respond to diverse tumor antigens. The use of immunotherapy in the treatment of cancer is based on the premise that tumors evade the endogenous immune response by being recognized as self and non-self. Tumors can escape immune surveillance and develop immune resistance using different mechanisms. Novel approaches in cancer immunotherapy are to counteract these resistance mechanisms, allowing the endogenous immune system to reject tumors. The recent success of immune-modulating agents in patients with refractory solid tumors has provided proof-of-concept of the efficacy of immune system activation as a immunotherapeutic modality, such as anti-PD-1 and anti-CTLA-4 treatments.

Despite those advances in immunotherapy, not all tumors respond to approved immune-modulating agents (Filley et al. 2017). Additionally, tumors generate a suppressive microenvironment to evade and inhibit the immune response which leads to variation of anti-cancer agents' effectiveness based on the unique patient characteristics (Topalian et al. 2014). The immuno-resistance to current immunotherapies remains a significant challenge. Accordingly, there is a need for additional therapies that can induce an immune response in the immunosuppressive environment targeting cancer and other malignancies. Combination immunotherapies with radiation, chemotherapy, and/or surgery can provide effective means of treatment for cancers. Novel druggable targets and treatment combinations will play a critical role in expanding the horizons of immunotherapy in the treatment of various cancers.

### BRIEF SUMMARY

Some aspects of the present disclosure are directed to an antibody or an antigen-binding portion thereof that specifically binds CD161, comprising a heavy chain variable region (VH) and a light chain variable region (VL); wherein the VH comprises a VH complementarity determining region 1 (VH-CDR1), a VH-CDR2, and a VH-CDR3; wherein the VL comprises a VL-CDR1, a VL-CDR2, and a VL-CDR3; and wherein the VH-CDR3 comprises an amino acid sequence selected from the sequences set forth in SEQ ID NOs: 3, 13, 23, 33, 43, 53, 63, 73, 83, 93, 103, 113, 123, 133, 143, 153, 163, 173, 183, 193, 203, 213, 223, 233, 243, 253, 263, 273, 283, 293, 303, 313, 323, and 333.

In some aspects, the VH-CDR2 comprises an amino acid sequence selected from the sequences set forth in SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, and 332. In some aspects, the VH-CDR1 comprises an amino acid sequence selected from the sequences set forth in SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, and 331. In some aspects, the VL-CDR3 comprises an amino acid sequence selected from the sequences set forth in SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, and 336. In some aspects, the VL-CDR2 comprises an amino acid sequence selected from the sequences set forth in SEQ ID NOs: 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, and 335. In some aspects, the VL-CDR1 comprises an amino acid sequence selected from the sequences set forth in SEQ ID NOs: 4, 14, 24, 34, 44, 54, 64, 74, 84, 94, 104, 114, 124, 134, 144, 154, 164, 174, 184, 194, 204, 214, 224, 234, 244, 254, 264, 274, 284, 294, 304, 314, 324, and 334.

In some aspects, the antibody or the antigen-binding portion thereof comprises: (i) a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6; (ii) a VH-CDR1 comprising the amino acid sequence set forth in SEQ ID NO: 91, a VH-CDR2 comprising the amino acid sequence set forth in SEQ ID NO: 92, a VH-CDR3 comprising the amino acid sequence set forth in SEQ ID NO: 93, a VL-CDR1 comprising the amino acid sequence set forth in SEQ ID NO: 94, a VL-CDR2 comprising the amino acid sequence set forth in SEQ ID NO: 95, and a VL-CDR3 comprising the amino acid sequence set forth in SEQ ID NO: 96; (iii) a VH-CDR1 comprising the amino acid sequence set forth in SEQ ID NO: 111, a VH-CDR2 comprising the amino acid sequence set forth in SEQ ID NO: 112, a VH-CDR3 comprising the amino acid sequence set forth in SEQ ID NO: 113, a VL-CDR1 comprising the amino acid sequence set forth in SEQ ID NO: 114, a VL-CDR2 comprising the amino acid sequence set forth in SEQ ID NO: 115, and a VL-CDR3 comprising the amino acid sequence set forth in SEQ ID NO: 116; (iv) a VH-CDR1 comprising the amino acid sequence set forth in SEQ ID NO: 141, a VH-CDR2 comprising the amino acid sequence set forth in SEQ ID NO: 142, a VH-CDR3 comprising the amino acid sequence set forth in SEQ ID NO: 143, a VL-CDR1 comprising the amino acid sequence set forth in SEQ ID NO: 144, a VL-CDR2 comprising the amino acid sequence set forth in SEQ ID NO: 145, and a VL-CDR3 comprising the amino acid sequence set forth in SEQ ID NO: 146; (v) a VH-CDR1 comprising the amino acid sequence set forth in SEQ ID NO: 211, a VH-CDR2 comprising the amino acid sequence set forth in SEQ ID NO: 212, a VH-CDR3 comprising the amino acid sequence set forth in SEQ ID NO: 213, a VL-CDR1 comprising the amino acid sequence set forth in SEQ ID NO: 214, a VL-CDR2 comprising the amino acid sequence set forth in SEQ ID NO: 215, and a VL-CDR3 comprising the amino acid sequence set forth in SEQ ID NO: 216; (vi) a VH-CDR1 comprising the amino acid sequence set forth in SEQ ID NO: 221, a VH-CDR2 comprising the amino acid sequence set forth in SEQ ID NO: 222, a VH-CDR3 comprising the amino acid sequence set forth in SEQ ID NO: 223, a VL-CDR1 comprising the amino acid sequence set forth in SEQ ID NO: 224, a VL-CDR2 comprising the amino acid sequence set forth in SEQ ID NO: 225, and a VL-CDR3 comprising the amino acid sequence set forth in SEQ ID NO: 226; (vii) a VH-CDR1 comprising the amino acid sequence set forth in SEQ ID NO: 271, a VH-CDR2 comprising the amino acid sequence set forth in SEQ ID NO: 272, a VH-CDR3 comprising the amino acid sequence set forth in SEQ ID NO: 273, a VL-CDR1 comprising the amino acid sequence set forth in SEQ ID NO: 274, a VL-CDR2 comprising the amino acid sequence set forth in SEQ ID NO: 275, and a VL-CDR3 comprising the amino acid sequence set forth in SEQ ID NO: 276; (viii) a VH-CDR1 comprising the amino acid sequence set forth in SEQ ID NO: 281, a VH-CDR2 comprising the amino acid sequence set forth in SEQ ID NO: 282, a VH-CDR3 comprising the amino acid sequence set forth in SEQ ID NO: 283, a VL-CDR1 comprising the amino acid sequence set forth in SEQ ID NO: 284, a VL-CDR2 comprising the amino acid sequence set forth in SEQ ID NO: 285, and a VL-CDR3 comprising the amino acid sequence set forth in SEQ ID NO: 286; (ix) a VH-CDR1 comprising the amino acid sequence set forth in SEQ ID NO: 291, a VH-CDR2 comprising the amino acid sequence set forth in SEQ ID NO: 292, a VH-CDR3 comprising the amino acid sequence set forth in SEQ ID NO: 293, a VL-CDR1 comprising the amino acid sequence set forth in SEQ ID NO: 294, a VL-CDR2 comprising the amino acid sequence set forth in SEQ ID NO: 295, and a VL-CDR3 comprising the amino acid sequence set forth in SEQ ID NO: 296; (x) a VH-CDR1 comprising the amino acid sequence set forth in SEQ ID NO: 301, a VH-CDR2 comprising the amino acid sequence set forth in SEQ ID NO: 302, a VH-CDR3 comprising the amino acid sequence set forth in SEQ ID NO: 303, a VL-CDR1 comprising the amino acid sequence set forth in SEQ ID NO: 304, a VL-CDR2 comprising the amino acid sequence set forth in SEQ ID NO: 305, and a VL-CDR3 comprising the amino acid sequence set forth in SEQ ID NO: 306; or (xi) any combination of (i) to (x).

In some aspects, the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 707, 317, 327, and 337. In some aspects, the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, and 338.

Some aspects of the present disclosure are directed to an antibody or an antigen-binding portion thereof that specifically binds CD161, comprising a variable heavy (VH) domain and a variable light (VL) domain, wherein the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 707, 317, 327, and 337; and wherein the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, and 338.

In some aspects, the VH comprises an amino acid sequence selected from SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 707, 317, 327, and 337. In some aspects, the VL comprises an amino acid sequence selected from SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, and 338.

In some aspects, (i) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 7; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 8; (ii) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 97; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 98; (iii) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 117; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 118; (iv) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 147; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 148; (v) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 217; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 218; (vi) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 227; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 228; (vii) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 277; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 278; (viii) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 287; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 288; (ix) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 297; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 298; (x) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 307; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 308; or (xi) any combination of (i) to (x).

In some aspects, the antibody or antigen-binding portion thereof comprises: (i) a VH comprising the amino acid sequence set forth in SEQ ID NO: 7, and a VL comprising the amino acid sequence set forth in SEQ ID NO: 8; (ii) a VH comprising the amino acid sequence set forth in SEQ ID NO: 97, and a VL comprising the amino acid sequence set forth in SEQ ID NO: 98; (iii) a VH comprising the amino acid sequence set forth in SEQ ID NO: 117, and a VL comprising the amino acid sequence set forth in SEQ ID NO: 118; (iv) a VH comprising the amino acid sequence set forth in SEQ ID NO: 147, and a VL comprising the amino acid sequence set forth in SEQ ID NO: 148; (v) a VH comprising the amino acid sequence set forth in SEQ ID NO: 217, and a VL comprising the amino acid sequence set forth in SEQ ID NO: 218; (vi) a VH comprising the amino acid sequence set forth in SEQ ID NO: 227, and a VL comprising the amino acid sequence set forth in SEQ ID NO: 228; (vii) a VH comprising the amino acid sequence set forth in SEQ ID NO: 277, and a VL comprising the amino acid sequence set forth in SEQ ID NO: 278; (viii) a VH comprising the amino acid sequence set forth in SEQ ID NO: 287, and a VL comprising the amino acid sequence set forth in SEQ ID NO: 288; (ix) a VH comprising the amino acid sequence set forth in SEQ ID NO: 297, and a VL comprising the amino acid sequence set forth in SEQ ID NO: 298; (x) a VH comprising the amino acid sequence set forth in SEQ ID NO: 307, and a VL comprising the amino acid sequence set forth in SEQ ID NO: 308; or (xi) any combination of (i) to (x). Some aspects of the present disclosure are directed to an antibody or antigen-binding portion thereof that binds the same epitope as an antibody or antigen-binding portion thereof disclosed herein. Some aspects of the present disclosure are directed to an antibody or antigen-binding portion thereof that cross-competes for binding CD161 with an antibody or antigen-binding portion thereof disclosed herein.

In some aspects, the antibody or antigen-binding portion thereof binds CD161 with a K_{D} of less than about 1000 nM, less than about 500 nM, less than about 100 nM, less than about 50 nM, or less than about 10 nM. In some aspects, the antibody or antigen-binding portion thereof binds CD161 with a K_{D} of less than about 50 nM. In some aspects, the antibody or antigen-binding portion thereof binds CD161 with a K_{D} of less than about 10 nM

In some aspects, the antibody or antigen-binding portion thereof inhibits the interaction between CD161 and C-type lectin domain family 2 member D (CLEC2D).

In some aspects, the antibody or antigen-binding portion thereof is capable of inducing or enhancing production of one or more cytokine by an immune cell. In some aspects, the one or more cytokine comprises IL2, TNFa, IFNg, or any combination thereof.

In some aspects, the antigen-binding portion of the antibody comprises a VHH, a vNAR, a microbody, a nanobody, an scFv, or any combination thereof.

Some aspects of the present disclosure are directed to a multi-specific antibody comprising an antibody or antigen-binding portion thereof disclosed herein. Some aspects of the present disclosure are directed to a bispecific antibody comprising an antibody or antigen-binding portion thereof disclosed herein.

Some aspects of the present disclosure are directed to a nucleic acid molecule or a set of nucleic acid molecules encoding an antibody or antigen-binding portion thereof disclosed herein, a multi-specific antibody disclosed herein, or a bispecific antibody disclosed herein.

Some aspects of the present disclosure are directed to a vector or a set of vectors comprising a nucleic acid molecule or a set of nucleic acid molecules disclosed herein. In some aspects, the vector is a viral vector.

Some aspects of the present disclosure are directed to a host cell comprising a nucleic acid molecule or a set of nucleic acid molecules disclosed herein or a vector or a set of vectors disclosed herein.

Some aspects of the present disclosure are directed to a pharmaceutical composition comprising an antibody or antigen-binding portion thereof disclosed herein, a multi-specific antibody disclosed herein, a bispecific antibody disclosed herein, a nucleic acid molecule or a set of nucleic acid molecules disclosed herein, a vector or a set of vectors disclosed herein, or a host cell disclosed herein and a pharmaceutically acceptable carrier.

Some aspects of the present disclosure are directed to a method of treating a disease or disorder in a subject in need thereof, comprising administering to the subject an antibody or antigen-binding portion thereof disclosed herein, a multi-specific antibody disclosed herein, a bispecific antibody disclosed herein, a nucleic acid molecule or a set of nucleic acid molecules disclosed herein, a vector or a set of vectors disclosed herein, a host cell disclosed herein, or a pharmaceutical composition disclosed herein. In some aspects, the disease or disorder comprises a cancer.

Some aspects of the present disclosure are directed to a method of inducing an immune response in a subject in need thereof, comprising administering to the subject an antibody or antigen-binding portion thereof disclosed herein, a multi-specific antibody disclosed herein, a bispecific antibody disclosed herein, a nucleic acid molecule or a set of nucleic acid molecules disclosed herein, a vector or a set of vectors disclosed herein, a host cell disclosed herein, or a pharmaceutical composition disclosed herein. In some aspects, the subject is afflicted with a cancer.

Some aspects of the present disclosure are directed to a method of treating a cancer in a subject in need thereof, comprising administering to the subject an antibody or antigen-binding portion thereof disclosed herein, a multi-specific antibody disclosed herein, a bispecific antibody disclosed herein, a nucleic acid molecule or a set of nucleic acid molecules disclosed herein, a vector or a set of vectors disclosed herein, a host cell disclosed herein, or a pharmaceutical composition disclosed herein.

In some aspects, the cancer is selected from acoustic neuroma, acute lymphocytic leukemia, acute myelocytic leukemia, adenocarcinoma, and cancer of the urinary system, and carcinomas, angiosarcoma, astrocytoma, basal cell carcinoma, bile duct carcinoma, biliary tract cancer, bladder cancer, bone cancer, brain cancer, brain stem glioma, breast cancer, bronchogenic carcinoma, Burkitt's lymphoma and marginal zone B cell lymphoma, cancer of the adrenal gland, cancer of the anal region, cancer of the digestive system, cancer of the endocrine system, cancer of the esophagus, cancer of the parathyroid gland, cancer of the penis, cancer of the respiratory system, cancer of the small intestine, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, central nervous system (CNS) cancer, cervical cancer, chondrosarcoma, chordoma, choriocarcinoma, chronic leukemia, chronic lymphocytic leukemia, chronic myelocytic (granulocytic) leukemia, colon carcinoma, colon sarcoma, colorectal cancer, connective tissue cancer, craniopharyngioma, cystadenocarcinoma, embryonal carcinoma, endometrial cancer, endotheliosarcoma, environmentally-induced cancers including those induced by asbestos, ependymoma, epidermoid cancer, epithelial carcinoma, esophageal cancer, esophageal carcinoma, Ewing's tumor, eye cancer, fibrosarcoma, gastric cancer, gastrointestinal cancer, germ cell tumor, glioblastoma (e.g. glioblastoma multiforme), glioma, head and neck cancer, heavy chain disease, hemangioblastoma, hepatoma, Hodgkin's disease, intraepithelial neoplasm, Kaposi's sarcoma, kidney cancer (e.g.renal cell carcinoma (RCC)), larynx cancer, leiomyosarcoma, leukemia, liposarcoma, liver cancer, lung cancer (small cell, large cell), lung carcinoma, lymphangioendotheliosarcoma, lymphangiosarcoma, mantle cell lymphoma, medullary carcinoma, medulloblastoma, melanoma, menangioma, mesothelioma, multiple myeloma, myeloblasts promyelocyte myelomonocytic monocytic erythroleukemia, myxosarcoma, nasopharyngeal carcinoma, neoplasm of the central nervous system (CNS), neuroblastoma, non-Hodgkin's disease, non-small cell lung cancer (NSCLC), non-small cell lung carcinoma, oligodendroglioma, oral cavity cancer (for example lip, tongue, mouth and pharynx), osteogenic sarcoma, osteosarcoma, ovarian cancer, pancreatic cancer, papillary adenocarcinomas, papillary carcinoma, pediatric sarcoma, pinealoma, pituitary adenoma, Polycythemia vera Lymphoma, primary CNS lymphoma, prostate cancer (e.g. hormone refractory prostate adenocarcinoma), rectal cancer, renal cancer (e.g. clear cell carcinoma), retinoblastoma, rhabdomyosarcoma, sarcoma, sarcoma of soft tissue, sebaceous gland carcinoma, seminoma, sinonasal natural killer, skin cancer, small-cell lung cancer (SCLC), solid tumors of childhood, spinal axis tumor, squamous cell cancer, squamous cell carcinoma, stomach cancer, sweat gland carcinoma, synovioma, testicular cancer, thyroid cancer, tumor angiogenesis, uterine cancer, virus-related cancers or cancers of viral origin (e.g. human papilloma virus (HPV-related or- originating tumors)), Waldenstrom's macroglobulinemia, and Wilm's tumor; and any combinations of said cancers.

Some aspects of the present disclosure are directed to a method of treating an infectious disease in a subject in need thereof, comprising administering to the subject an antibody or antigen-binding portion thereof disclosed herein, a multi-specific antibody disclosed herein, a bispecific antibody disclosed herein, a nucleic acid molecule or a set of nucleic acid molecules disclosed herein, a vector or a set of vectors disclosed herein, a host cell disclosed herein, or a pharmaceutical composition disclosed herein. In some aspects, the infectious disease comprises: (i) infection by Influenza, Herpes, Giardia, Malaria, Leishmania, or any combination thereof; (ii) infection by human immunodeficiency virus (HIV), Hepatitis virus herpes virus, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus, or arboviral encephalitis virus, or any combination thereof; (iii) infection by chlamydia, rickettsial bacteria, mycobacteria, staphylococci, streptococci, pneumonococci, meningococci, conococci, klebsiella, proteus, serratia, pseudomonas, legionella, diphtheria, salmonella, bacilli, cholera, tetanus, botulism, anthrax, plague, leptospirosis, and Lyme's disease bacteria, or any combination thereof; (iv) infection by Candida, Cryptococcus neoformans, Aspergillus, genus Mucorales, Sporothrix schenkii, Blastomyces dermatitidis, Paracoccidioides brasiliensis, Coccidioides immitis, or Histoplasma capsulatum, or any combination thereof; (v) infection by Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, or Nippostrongylus brasiliensis, or any combination thereof; or (vi) any combination of (i) to (v).

Some aspects of the present disclosure are directed to a method of treating an autoimmune disease in subject in need thereof, comprising administering to the subject an antibody or antigen-binding portion thereof disclosed herein, a multi-specific antibody disclosed herein, a bispecific antibody disclosed herein, a nucleic acid molecule or a set of nucleic acid molecules disclosed herein, a vector or a set of vectors disclosed herein, a host cell disclosed herein, or a pharmaceutical composition disclosed herein.

Some aspects of the present disclosure are directed to a method of activating an immune cell, comprising contacting the immune cell with an antibody or antigen-binding portion thereof disclosed herein, a multi-specific antibody disclosed herein, a bispecific antibody disclosed herein, a nucleic acid molecule or a set of nucleic acid molecules disclosed herein, a vector or a set of vectors disclosed herein, a host cell disclosed herein, or a pharmaceutical composition disclosed herein.

### BRIEF DESCRIPTION OF THE DRAWINGS/FIGURES

FIG. 1 is a violin plot of scRNA-seq gene expression profiles of Killer cell lectin-like receptor B1 protein (KLRB1/CD161), PDCD1, LAG3, CTLA4, HAVCR2, and TIGIT in CD4 T cells, CD8 T cells, dendritic cells (DC), macrophages, monocytes, naïve B-cells, NK cells, and regulatory T cells (Tregs) from specific tumor samples, as indicated.

FIG. 2 is a violin plot of scRNA-seq gene expression of CD161, PDCDI, LAG3, CTLA4, HAVCR2, and TIGIT in naïve-like CD8 T cells, early active CD8 T cells, effector memory CD8 T cells, CD8 Tpex cells, and CD8 Tex cells for specific tumor samples, as indicated.

FIG. 3 is a graphical representation of cell-based binding of anti-CD161-positive hits/antibodies cross-reactive to human and cyno CD161 proteins expressed on CHOK1 cells as measured by flow cytometry.

FIG. 4 is a graphical representation of binding of specific anti-CD161 antibodies to human CD161 (hCD161) protein in an ELISA assay.

FIGS. 5A-5C are FACS plots of cell-based binding with isotype control (FIG. 5A) or anti-CD161 positive antibodies (01D17, FIG. 5B; and 03K18, FIG. 5C). FIG. 5D is a graphical representation of binding of the various anti-CD161 antibodies to human CD161 expressing CHOK1 cells.

FIG. 6 is a graphical representation of binding of the human CLEC2D multimer with human CD161 expressing CHOK1 cells. HP-3G10, anti-CD161 reference monoclonal antibody, that blocks hCLEC2D multimer binding with hCD161 expressing CHOK1 cells.

FIG. 7 is a bar graph illustrating the percent blocking of hCD161: hCLEC2D interaction of anti-CD161 antibodies using a cell-based assay.

FIG. 8 is a graphical representation of an ELISA assay showing concentration-dependent inhibition of the interaction of hCD161: hCLE2CD by various anti-CD161 antibodies.

FIG. 9 is a bar graph illustrating IL-2 cytokine release in SEB (Staphylococcal enterotoxin B) stimulated healthy human donors PBMCs following contact with various anti-CD161 antibodies, as measured by ELISA.

FIG. 10 is a bar graph illustrating IL-2 cytokine release in hCD161 expressing Jurkat cells activated by T cell engager with Raji cells following treatment with various anti-CD161 antibodies, as measured by ELISA.

FIG. 11 is a schematic representation TCR activation impacted by CD161-CLEC2D interaction, using Jurkat-MART-1 TCR cells and MeWo cells as an example.

FIG. 12 is a graphical representation of TCR activation (as measured by IL-2 release, pg/mL) in MART1 TCR-specific T cells contacted with hCLEC2D-GFP overexpressing MeWo cells (HLA; A*0201) in the presence of varying concentrations of an anti-CD161 antibody described herein. The top dashed line (1) represents the level of IL-2 release when Jurkat-MART1 TCR CD161 cells are contacted with mock (GFP) MeWo cells (control, no inhibition of TCR activity). The lower dashed line (2) represents the level of IL2 release when Jurkat-MART1 TCR CD161 cells are contacted with hCLEC2D-GFP overexpressing MeWo cells in the absence of an anti-CD161 antibody (control, inhibition of TCR activity).

FIG. 13 is a graphical representation of dose-dependent anti-CD161 antibody binding to TALL-104 effector cells, which express CD161.

FIGS. 14A-14C provide a schematic representation of a cytotoxicity assay (FIG. 14A) and preliminary data showing that the cytotoxicity assay is capable of detecting a change in TALL-104 cytolysis of MeWo cells (FIG. 14B) and PC3 cells (FIG. 14C).

FIG. 15 is a graphical representation of TALL-104 effector cell cytolysis (%) of human CLEC2D overexpressing-PC3 target cells in the presence of increasing doses of an anti-CD161 monoclonal antibody. The top dashed line (1) represents the level of TALL-104 cytolysis (%) of MOCK-PC3 cells (which do not express human CLEC2D, and thus do not inhibit the TALL-104 effector cells). The lower dashed line (2) represents the level of TALL-104 cytolysis (%) of CLEC2D overexpressing-PC3 target cells, in the absence of an anti-CD161 antibody.

FIGS. 16A-16B are dot plots showing the relative tumor killing capacity from the coculture of primary NK cells with PC-3 CLEC2D-OE cells (overexpressing CLEC2D; FIG. 16A) or Raji cells (FIG. 16B) in the presence of anti-CD161 mAbs. * p<0.05, **** p<0.00001, as determined by one-way ANOVA with multiple comparisons.

FIG. 17 is a graphical representation of PC3-CLEC2D-OE tumor volume (mm³) in NSG mice following two intratumoral injections of NK cells and administration of vehicle or the anti-CD161 antibody 12G06. n=4 per group. * p≤0.05, as determined by two-way ANOVA.

FIG. 18 is a graphical representation of U87 tumor volume (mm³) in NSG mice following co-implantation of human PBMCs and administration of vehicle or the anti-CD161 antibody 12G06. n=4 per group. * p≤0.05, as determined by two-way ANOVA.

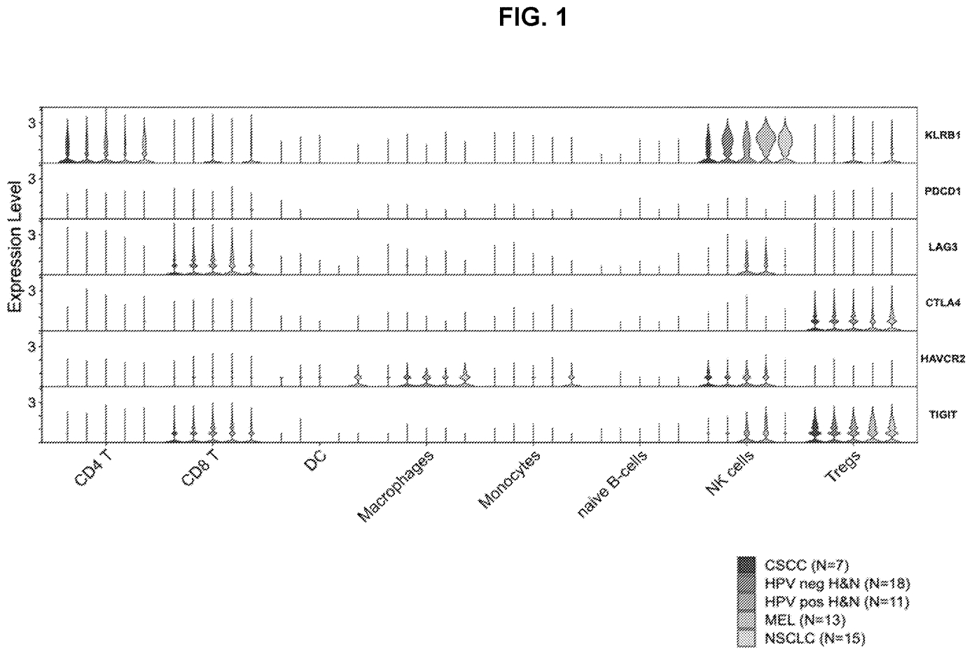

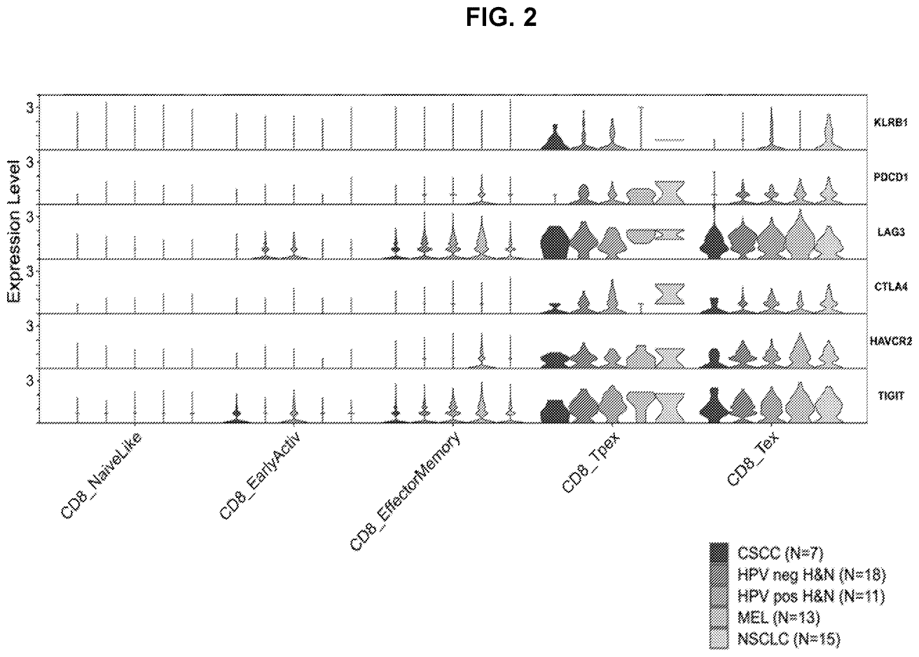

### DETAILED DESCRIPTION

The present disclosure relates to antibodies and antigen-binding portions thereof that specifically bind CD161, referred to herein as anti-CD161 antibodies. Some aspects of the present disclosure are directed to bispecific or multispecific antibodies comprising the anti-CD161 antibody. Other aspects of the present disclosure are directed to methods of treating a disease or disorder in a subject in need thereof comprising administering an antibody or an antigen-binding portion thereof described herein to the subject.

Before the present disclosure is described in greater detail, it is to be understood that this disclosure is not limited to the particular compositions or process steps described, which, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting.

### I. Terms

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “antibody” refers, in some aspects, to a protein comprising at least two heavy (H) chains and two light (L) chains inter-connected by disulfide bonds or an antigen-binding portion thereof. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. A heavy chain may have the C-terminal lysine or not. Unless specified otherwise herein, the amino acids in the variable regions are numbered using the Kabat numbering system and those in the constant regions are numbered using the EU system.

An “IgG antibody”, e.g., a human IgG1, IgG2, IgG3 and IgG4 antibody, as used herein has, in some aspects, the structure of a naturally-occurring IgG antibody, i.e., it has the same number of heavy and light chains and disulfide bonds as a naturally-occurring IgG antibody of the same subclass. For example, an anti-CD161 IgG1, IgG2, IgG3 or IgG4 antibody consists of two heavy chains (HCs) and two light chains (LCs), wherein the two HCs and LCs are linked by the same number and location of disulfide bridges that occur in naturally-occurring IgG1, IgG2, IgG3 and IgG4 antibodies, respectively (unless the antibody has been mutated to modify the disulfide bridges).

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷M or less, 10⁻⁸M or less, 5×10⁻⁹M or less, or between 10⁻⁸ M and 10⁻¹⁰ M or less, but does not bind with high affinity to unrelated antigens.

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In some aspects, the anti-CD161 antibodies described herein are of the IgG1 subtype. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen (e.g., human CD161). It has been shown that the antigen-binding function of an antibody can be performed by fragments of a full-length antibody. Examples of binding fragments encompassed within the term “antigen-binding portion” of an antibody, e.g., an anti-CD161 antibody described herein, include (i) a Fab fragment (fragment from papain cleavage) or a similar monovalent fragment consisting of the V_{L}, V_{H}, LC and CH1 domains; (ii) a F(ab′) 2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

A “bispecific antibody” or “bifunctional antibody” is an artificial hybrid antibody having two different heavy/light chain pairs and two different binding sites. A “multispecific antibody” or “multifunctional antibody” is an artificial hybrid antibody having more than two different heavy/light chain pairs and two different binding sites. Bispecific and multispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “monoclonal antibody,” as used herein, refers to an antibody from a population of substantially homogeneous antibodies, i.e., the individual antibodies comprised in the population are substantially similar and bind the same epitope(s) (e.g., the antibodies display a single binding specificity and affinity), except for possible variants that may arise during production of the monoclonal antibody, such variants generally being present in minor amounts. The modifier “monoclonal” indicates the character of the antibody as being obtained from a substantially homogeneous population of antibodies, and is not to be construed as requiring production of the antibody by any particular method. The term “human monoclonal antibody” refers to an antibody from a population of substantially homogeneous antibodies that display(s) a single binding specificity, and which has variable and optional constant regions derived from human germline immunoglobulin sequences. In some aspects, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences encoded by the germline genes, but include subsequent rearrangements and mutations which occur, for example, during antibody maturation. As known in the art (see, e.g., Lonberg (2005) Nature Biotech. 23 (9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen cannot have sequence identity with the original nucleic acid molecules, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The anti-CD161 antibodies described herein can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized” antibody refers to an antibody in which some, most or all of the amino acids outside the CDR domains of a non-human antibody are replaced with corresponding amino acids derived from human immunoglobulins. In some aspects of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

The phrases “an antibody recognizing an antigen” and “an antibody specific for an antigen” are used interchangeably herein with the term “an antibody which binds specifically to an antigen.”

An “isolated antibody,” as used herein, is intended to refer to an antibody which is substantially free of other proteins and cellular material.

As used here, the term “CD161” refers to a polypeptide encoded by the human KLRB1 gene, which is expressed in a subset of NK cells and in peripheral blood T cells. CD161 can also be referred to as “killer cell lectin-like receptor subfamily B member 1,” “KLRB1,” “C-type lectin domain family 5 member B,” “CLEC5B,” “C-type lectin domain family 5 member B,” and “NKRP1a.” CD161 is believed to inhibit NK cell-mediated cytotoxicity and interferon-gamma secretion in target cells by binding CLEC2D/LLT1. Activated CD161 leads to specific acid sphingomyelinase (aSMase) stimulation, with subsequent marked elevation of intracellular ceramide.CD161 acts as a lectin that binds to the terminal carbohydrate Gal-alpha (1,3) Gal epitope as well as to the N-acetyllactosamine epitope. The canonical human CD161 sequence is provided in Table 1 (UniProt Q12918).

TABLE 1
Canonical human CD161 amino acid sequence.

| | |
|---|---|
| Human CD161 | MDQQAIYAELNLPTDSGPESSSPSSLPRDVCQGSPWHQFALKLSCAGIILLVLVVTGLSVSV |
| (UniProt KB- | TSLIQKSSIEKCSVDIQQSRNKTTERPGLLNCPIYWQQLREKCLLFSHTVNPWNNSLADCST |
| Q12918) | KESSLLLIRDKDELIHTQNLIRDKAILFWIGLNFSLSEKNWKWINGSFLNSNDLEIRGDAKE |
| | NSCISISQTSVYSEYCSTEIRWICQKELTPVRNKVYPDS (SEQ ID NO: 341) |

As used herein, an antibody that inhibits “CD161 activity” is intended to refer to an antibody that inhibits or reduces one or more activity of CD161. In some aspects, an anti-CD161 antibody disclosed herein inhibits or reduces the interaction between human CD161 and CLEC2D. In some aspects, an anti-CD161 antibody disclosed herein increases NK cell-mediated cytotoxicity, i.e., by removing CD161-mediated inhibition. In some aspects, an anti-CD161 antibody disclosed herein increases target cell interferon-gamma secretion, i.e., by removing CD161-mediated inhibition. In some aspects, an anti-CD161 antibody disclosed herein inhibits or reduces specific acid sphingomyelinase (aSMase) stimulation in a target cell.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (Clq) of the classical complement system. Thus, an Fc region comprises the constant region of an antibody excluding the first constant region immunoglobulin domain (e.g., CHI or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally-occurring Fc).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally-occurring variants thereof. Native sequence Fc include the various allotypes of Fcs (see, e.g., Jefferis et al. (2009) mAbs 1:1).

The term “epitope” or “antigenic determinant” refers to a site on an antigen (e.g., CD161) to which an immunoglobulin or antibody specifically binds, e.g., as defined by the specific method used to identify it. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., from CD161) are tested for reactivity with a given antibody (e.g., anti-CD161 antibody). Methods of determining spatial conformation of epitopes include techniques in the art and those described herein, for example, x-ray crystallography, x-ray co-crystallography, antigen mutational analysis, 2-dimensional nuclear magnetic resonance and HDX-MS (see, e.g., Epitope Mapping Protocols in Methods in Molecular Biology, Vol. 66, G. E. Morris, Ed. (1996)).

The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the “same epitope on CD161” with the antibodies described herein include, for example, epitope mapping methods, such as, x-ray analyses of crystals of antigen: antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2, and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis. In some aspects, an antibody competes with, and inhibits binding of another antibody to a target by at least 50%, 60%, 70%, 80%, 90% or 100%. The level of inhibition or competition can be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999. Two antibodies “cross-compete” if antibodies block each other both ways by at least 50%, i.e., regardless of whether one or the other antibody is contacted first with the antigen in the competition experiment.

Competitive binding assays for determining whether two antibodies compete or cross-compete for binding include: competition for binding to cells expressing CD161, e.g., by flow cytometry, such as described in the Examples. Other methods include: SPR (e.g., BIACORE®), solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25 (1): 7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 instrument using the predetermined antigen, e.g., recombinant human CD161, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to CD161” refers to an antibody that binds to CD161 with a K_{D} of 10⁻⁷M or less, such as approximately less than 10⁻⁸ M, 10⁻⁹ M or 10⁻¹⁰ M or even lower. In some aspects, such antibodies that do not cross-react with CD161 from a non-human species exhibit essentially undetectable binding against these proteins in standard binding assays.

The term “k_{assoc}” or “kₐ”, as used herein, is intended to refer to the association rate of a particular antibody-antigen interaction, whereas the term “k_{dis}” or “kₐ,” as used herein, is intended to refer to the dissociation rate of a particular antibody-antigen interaction. The term “K_{D}”, as used herein, is intended to refer to the dissociation constant, which is obtained from the ratio of kₐ to kₐ (i.e., kₐ/kₐ) and is expressed as a molar concentration (M). K_{D} values for antibodies can be determined using methods well established in the art. Available methods for determining the K_{D} of an antibody include surface plasmon resonance, a biosensor system such as a BIACORE® system or flow cytometry and Scatchard analysis.

As used herein, the term “high affinity” for an IgG antibody refers to an antibody having a K_{D} of 10⁻⁸M or less, 10⁻⁹M or less, or 10⁻¹⁰ M or less for a target antigen. However, “high affinity” binding can vary for other antibody isotypes. For example, “high affinity” binding for an IgM isotype refers to an antibody having a K_{D} of 10⁻¹⁰ M or less, or 10⁻⁸ M or less.

The term “EC₅₀” in the context of an in vitro or in vivo assay using an antibody or antigen binding fragment thereof, refers to the concentration of an antibody or an antigen-binding portion thereof that induces a response that is 50% of the maximal response, i.e., halfway between the maximal response and the baseline.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in an anti-CD161 antibody is replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12 (10): 879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See worldwideweb.ncbi.nlm.nih.gov.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, “administering” refers to the physical introduction of a composition comprising a therapeutic agent to a subject, using any of the various methods and delivery systems known to those skilled in the art. Different routes of administration for the anti-CD161 antibodies described herein include intravenous, intramuscular, subcutaneous, intraperitoneal, intravesical, transdermal, spinal or other parenteral routes of administration, for example by injection or infusion. In some aspects, the administration comprises a parental administration. As used herein, “parenteral administration” means modes of administration other than enteral and topical administration, usually by injection, and includes, without limitation, intravenous, intraocular, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease, such as cancer, that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, dog, cow, chickens, amphibians, reptiles, etc.

As used herein, the terms “ug” and “uM” are used interchangeably with “ug” and “uM,” respectively.

Various aspects described herein are described in further detail in the following subsections.

### II. Compositions of the Disclosure

The present disclosure relates to antibodies and antigen-binding portions thereof that specifically bind CD161. In some aspects, the anti-CD161 antibodies and antigen-binding portions thereof described herein block or reduce the interaction between CD161 and CLEC2D. Binding of CD161 to CLEC2D inhibits T cell activation. As such, in some aspects, the anti-CD161 antibodies and antigen-binding portions thereof described herein are capable of enhancing an immune response in a human subject but blocking or reducing CD161-CLEC2D induced inhibition of T cell activation.

Any method can be used to determine the ability of an anti-CD161 to overcome the inhibition of T cell activation mediated by CD161-CLEC2D interaction. The anti-CD161 antibodies described herein induce or enhance cytokine production by an immune cell, for example, as determined by a cytokine assay. In some aspects, the cytokine assay determines an amount of at least one cytokine secreted from an immune cell contacted with the anti-CD161 antibody, wherein an increase in the amount of the at least one cytokine indicates induction or enhancement of cytokine production by the anti-CD161 antibody.

In some embodiments, an increase in cytokine production is at least 1.5 fold, 2 fold, 3 fold, 4 fold, 5 fold, 6 fold, 7 fold, 8 fold, 9 fold, or 10 fold higher compared to a control antibody (e.g., an equivalent antibody isotype that does not bind to CD161, e.g., and antibody that does not induce cytokine production). Comparing the amount of at least one cytokine produced by the immune cell to an amount secreted from a reference immune cell, wherein the reference immune cell is contacted with a control antibody, and wherein an increase in the amount of the at least one cytokine produced from the immune cell relative to the reference immune cell indicates induced or enhanced cytokine production resulting from blocking of the CD161-CLEC2D interaction. In some embodiments, the immune cell is a T cell either CD8+ or CD4+ T cell.

In some aspects, the anti-CD161 antibodies and antigen-binding portions thereof induce or enhance cytokine production by an immune cell. In some aspects, cytokine production by an immune cell contacted with the anti-CD161 antibodies and antigen-binding portions thereof is increased by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10, as compared to an immune cell prior to contacting or contacted with a control antibody (e.g., an equivalent antibody isotype that does not bind to CD161). In some aspects, the immune cell is a T cell. In some aspects the T cell is a CD8+ T cell or a CD4+ T cell. In some aspects, the immune cell is an NK cell.

In some aspects, contacting an immune cell with the anti-CD161 antibodies and antigen-binding portions thereof described herein results in increased expression of interleukin-2 (IL-2) by the immune cell. In some aspects, IL-2 expression is increased by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10, as compared to the expression of IL-2 by an immune cell prior to contacting or contacted with a control antibody.

In some aspects, contacting an immune cell with the anti-CD161 antibodies and antigen-binding portions thereof described herein results in increased expression of interferon-gamma (IFNg) by the immune cell. In some aspects, IFNg expression is increased by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10, as compared to the expression of IFNg by an immune cell prior to contacting or contacted with a control antibody.

In some aspects, contacting an immune cell with the anti-CD161 antibodies and antigen-binding portions thereof described herein results in increased expression of tumor necrosis factor-alpha (TNF-a) by the immune cell. In some aspects, TNF-a expression is increased by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10, as compared to the expression of TNF-a by an immune cell prior to contacting or contacted with a control antibody.

In some aspects, anti-CD161 antibodies and antigen-binding portions thereof described herein bind to human CD161 with high affinity, for example, with a K_{D} of 10⁻⁶M or less, 10⁻⁷M or less, 10⁻⁸ M or less, 10⁻⁹M or less, 10⁻¹⁰ M or less, 10⁻¹¹ M or less, 10⁻¹² M or less, 10⁻¹² M to 10⁻⁷ M, 10⁻¹¹ M to 10⁻⁷ M, 10⁻¹⁰ M to 10⁻⁷ M, or 10⁻⁹ M to 10⁻⁷ M. In some aspects, anti-CD161 antibodies and antigen-binding portions thereof described herein binds to human CD161, e.g., as determined by Surface Plasmon Resonance, e.g., using BIACORE™, with a K_{D} of 10⁻⁶ M or less, 10⁻⁷ M or less, 10⁻⁸ M or less, 10⁻⁹M (1 nM) or less, 10⁻¹⁰ M or less, 10⁻¹² M to 10⁻⁷M, 10⁻¹¹ M to 10⁻⁷M, 10⁻¹⁰ M to 10⁻⁷M, 10⁻⁹ M to 10⁻⁷M, or 10⁻⁸ M to 10⁻⁷M.

### II.A. Anti-CD161 Antibodies

In some aspects, the antibody or antigen-binding portion thereof comprises a heavy chain and a light chain, wherein the heavy chain comprises a heavy chain variable region (VH), and wherein the light chain comprises a light chain variable region (VL); wherein the VH comprises a VH complementarity determining region 1 (VH-CDR1), a VH-CDR2, and a VH-CDR3; wherein the VL comprises a VL-CDR1, a VL-CDR2, and a VL-CDR3; and wherein the VH-CDR3 comprises an amino acid sequence selected from the sequences set forth in SEQ ID NOs: 3, 13, 23, 33, 43, 53, 63, 73, 83, 93, 103, 113, 123, 133, 143, 153, 163, 173, 183, 193, 203, 213, 223, 233, 243, 253, 263, 273, 283, 293, 303, 313, 323, and 333. In some aspects, the VH-CDR2 comprises an amino acid sequence selected from the sequences set forth in SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, and 332. In some aspects, the VH-CDR1 comprises an amino acid sequence selected from the sequences set forth in SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, and 331. In some aspects, the VL-CDR3 comprises an amino acid sequence selected from the sequences set forth in SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, and 336. In some aspects, the VL-CDR2 comprises an amino acid sequence selected from the sequences set forth in SEQ ID NOs: 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, and 335. In some aspects, the VL-CDR1 comprises an amino acid sequence selected from the sequences set forth in SEQ ID NOs: 4 SEQ ID NOs: 4, 14, 24, 34, 44, 54, 64, 74, 84, 94, 104, 114, 124, 134, 144, 154, 164, 174, 184, 194, 204, 214, 224, 234, 244, 254, 264, 274, 284, 294, 304, 314, 324, and 334.

TABLE 2
Anti-CD161 Antibody Sequences

| SEQ | | | |
|---|---|---|---|
| ID | | | |
| NO | Antibody | Region | Sequence |
| 1 | 01C07 | VH-CDR1 | SNYWT |
| 2 | 01C07 | VH-CDR2 | YIYYIGTTNYNPSLKS |
| 3 | 01C07 | VH-CDR3 | ARDGGYSGTYWGLDP |
| 4 | 01C07 | VL-CDR1 | RASQSVSSSHLA |
| 5 | 01C07 | VL-CDR2 | GASSRAT |
| 6 | 01C07 | VL-CDR3 | QQYGSSPIT |
| 7 | 01C07 | VH | QVQLQESGPGLVKPSETLSLTCTVSGGSISSNYWTWIRQPPGKGLEWIG |
| | | | YIYYIGTTNYNPSLKSRVTISLDTSKNHFSLRLSSVTAADTAVYYCARD |
| | | | GGYSGTYWGLDPWGQGTLVTVSS |
| 8 | 01C07 | VL | EIVLTQSPGTLSLSPGERATLSCRASQSVSSSHLAWYQQKPGQAPRLLI |
| | | | YGASSRATGIPDRFSGSGSGTDFTLTISRLEPEDFAVYNCQQYGSSPIT |
| | | | FGQGTRLEIK |
| 11 | 01D17 | VH-CDR1 | SYYWS |
| 12 | 01D17 | VH-CDR2 | YVYNSESTYYNPSLKS |
| 13 | 01D17 | VH-CDR3 | DGGIVGLTAAFDI |
| 14 | 01D17 | VL-CDR1 | RASQSVSSNYLA |
| 15 | 01D17 | VL-CDR2 | GASSRAT |
| 16 | 01D17 | VL-CDR3 | QQYVNSPIT |
| 17 | 01D17 | VH | QVHLQESGPGLVKPSETLSLTCTVSGGSIISYYWSWIRQPPGKGLEWIG |
| | | | YVYNSESTYYNPSLKSRVTISEDTSKSQFSLKLSSVTAADTAVYYCARD |
| | | | GGIVGLTAAFDIWGQGTMVTVSS |
| 18 | 01D17 | VL | EIVLTQSPGTLSLSPGERATLSCRASQSVSSNYLAWYQQKPGQAPRLLI |
| | | | YGASSRATGIPDRFSGNGSGTDFTLTISRLEPEDFAVYYCQQYVNSPIT |
| | | | FGPGTKVDIK |
| 21 | 02I22 | VH-CDR1 | SYYWS |
| 22 | 02I22 | VH-CDR2 | YIYYIGSTNYNPSLKS |
| 23 | 02I22 | VH-CDR3 | DRMGALDY |
| 24 | 02I22 | VL-CDR1 | RSSQSLVHSDGNTYLS |
| 25 | 02I22 | VL-CDR2 | KISNRFS |
| 26 | 02I22 | VL-CDR3 | MQAAQFPT |
| 27 | 02I22 | VH | QVQLQESGPGLVKPSETLSLTCTVSGGSISSYYWSWIRQPPGKGLEWIG |
| | | | YIYYIGSTNYNPSLKSRVTISGDTSKNQFSLKLTSVTAADTAVYYCARD |
| | | | RMGALDYWGQGTLVTVSS |
| 28 | 02I22 | VL | DIVMTQTPLSSPVTLGQPASISCRSSQSLVHSDGNTYLSWLQQRPGQPP |
| | | | RLLIYKISNRFSGVPDRFSGSGAGTDFTLIISRVEAEDVGVYYCMQAAQ |
| | | | FPTFGGGTKVEIK |
| 31 | 02L14 | VH-CDR1 | SYYWS |
| 32 | 02L14 | VH-CDR2 | RIYTIGTTNYNPSLKS |
| 33 | 02L14 | VH-CDR3 | DPLGSFFDY |
| 34 | 02L14 | VL-CDR1 | TGTSSDVGGYNYVS |
| 35 | 02L14 | VL-CDR2 | EVSKRPS |
| 36 | 02L14 | VL-CDR3 | SSYAGSNNLV |
| 37 | 02L14 | VH | QVQLQESGPGLVKPSETLSLTCTVSGGSISSYYWSWIRQPAGKGLEWIG |
| | | | RIYTIGTTNYNPSLKSRVTMSVDTSKNQFSLKLSSVTAADTAVYYCARD |
| | | | PLGSFFDYWGQGSLVTVSS |
| 38 | 02L14 | VL | QSALTQPPSASGSPGQSVTISCTGTSSDVGGYNYVSWYQQHPGKAPKLM |
| | | | IYEVSKRPSGVPDRFSGSKSGNTASLTVSGLQAEDEADYYCSSYAGSNN |
| | | | LVFGGGTKLTVL |
| 41 | 03G19 | VH-CDR1 | SYYWS |
| 42 | 03G19 | VH-CDR2 | RTYTSGSTNYNPSLKS |
| 43 | 03G19 | VH-CDR3 | DPLGTFFDY |
| 44 | 03G19 | VL-CDR1 | TGTSSDVGGYNYVS |
| 45 | 03G19 | VL-CDR2 | EVSKRPS |
| 46 | 03G19 | VL-CDR3 | SSYAGINNVV |
| 47 | 03G19 | VH | QVQLQESGPGLVKPSETLSLTCTVSGDSINSYYWSWIRQPAGKGLEWIG |
| | | | RTYTSGSTNYNPSLKSRVTMSVDTSKKQISLKLSSVTAADTAVYYCARD |
| | | | PLGTFFDYWGQGILVTVSS |
| 48 | 03G19 | VL | QSALTQPPSASGSPGQSVTISCTGTSSDVGGYNYVSWYQQHPGKAPKFM |
| | | | IYEVSKRPSGVPDRFSGSKSGNTASLTVSGLQAEDEADYYCSSYAGINN |
| | | | VVFGGGTKLTVL |
| 51 | 03K18 | VH-CDR1 | SYYWS |
| 52 | 03K18 | VH-CDR2 | YIYYIGTTNYNPSLKS |
| 53 | 03K18 | VH-CDR3 | DRMGALDY |
| 54 | 03K18 | VL-CDR1 | RSSQSLVHSDGNTYLS |
| 55 | 03K18 | VL-CDR2 | EISNRFS |
| 56 | 03K18 | VL-CDR3 | MQAAQFPT |
| 57 | 03K18 | VH | QVQLQESGPGLVKPSETLSLTCTVSGGSISSYYWSWIRQPPGKGLEWIG |
| | | | YIYYIGTTNYNPSLKSRVTISVDTSKNQFSLKLSSVTAADTAVYYCARD |
| | | | RMGALDYWGQGTLVTVSS |
| 58 | 03K18 | VL | DIVMTQTPLSSPVTLGQPASISCRSSQSLVHSDGNTYLSWLQQRPGQPP |
| | | | RLLIYEISNRFSGVPDRFSGSGAGTDFTLKISRVEAEDVGVYYCMQAAQ |
| | | | FPTFGGGTKVEIK |
| 61 | 04D23 | VH-CDR1 | NYYWS |
| 62 | 04D23 | VH-CDR2 | RIYTSGSNNYNPSLKS |
| 63 | 04D23 | VH-CDR3 | DPLGTFFDY |
| 64 | 04D23 | VL-CDR1 | TGTSSDVGGYNYVS |
| 65 | 04D23 | VL-CDR2 | EVSKRPS |
| 66 | 04D23 | VL-CDR3 | SSYADTNNVV |
| 67 | 04D23 | VH | QVQLQESGPGLVKPSETLSLTCTVSGGSISNYYWSWIRQPAGKGLEWIG |
| | | | RIYTSGSNNYNPSLKSRVTMSVDTSKKQFSLKLSSVTAADTAVYYCARD |
| | | | PLGTFFDYWGQGTLVTVSS |
| 68 | 04D23 | VL | QSALTQPPSASGSPGQSVTISCTGTSSDVGGYNYVSWYQQHPGKAPKLM |
| | | | IYEVSKRPSGVPDRFSGSKSGNTASLTVSGLQAEDEADYYCSSYADTNN |
| | | | VVFGGGTKLTVL |
| 71 | 04I09 | VH-CDR1 | SYFWS |
| 72 | 04109 | VH-CDR2 | YIYYNGHTNYNPSLKS |
| 73 | 04109 | VH-CDR3 | DSGPWVPLDY |
| 74 | 04109 | VL-CDR1 | RASQSVISSYLA |
| 75 | 04I09 | VL-CDR2 | GASSRAT |
| 76 | 04109 | VL-CDR3 | QQYGSSPPT |
| 77 | 04I09 | VH | QVQLQESGPGLVKPSETLSLTCTVSGGSISSYFWSWIRQPPGKGLEWIG |
| | | | YIYYNGHTNYNPSLKSRVTISVDTSKNQFSLKLSSVTAADTAVYYCARD |
| | | | SGPWVPLDYWGQGTLVTVSS |
| 78 | 04109 | VL | EIVLTQSPGTLSLSPGERATLSCRASQSVISSYLAWYQQKPGQAPRLLI |
| | | | YGASSRATGIPDRFSGSGSGTDFTLTISRLEPEDFAVYFCQQYGSSPPT |
| | | | FGQGTKVEIK |
| 81 | 04L21 | VH-CDR1 | FYYWS |
| 82 | 04L21 | VH-CDR2 | YIYHSGSTNYNPSLKS |
| 83 | 04L21 | VH-CDR3 | DGGIVGVTEAIDI |
| 84 | 04L21 | VL-CDR1 | RASQVISSSYLA |
| 85 | 04L21 | VL-CDR2 | GASSRAT |
| 86 | 04L21 | VL-CDR3 | QQYGSSPLT |
| 87 | 04L21 | VH | QVQLQESGPGLVKPSETLSLTCTVSGGSISFYYWSWIRQPPGKGLEWIG |
| | | | YIYHSGSTNYNPSLKSRVTISVDTSKNQFSLKLSSVTAADTAVYYCARD |
| | | | GGIVGVTEAIDIWGQGTVVTVSS |
| 88 | 04L21 | VL | EVVLTQTPGTLSLSPGERATLSCRASQVISSSYLAWYQQKPGQAPRLLI |
| | | | FGASSRATGIPDRFSGSGSGTDFTLTISRLEPADFAVYYCQQYGSSPLT |
| | | | FGGGTKVEIK |
| 91 | 05D04 | VH-CDR1 | SYYWS |
| 92 | 05D04 | VH-CDR2 | YIYYSGSANYNPSLKS |
| 93 | 05D04 | VH-CDR3 | ASDRGPWGGGFDY |
| 94 | 05D04 | VL-CDR1 | RASQSVSSSYLA |
| 95 | 05D04 | VL-CDR2 | GASSRAT |
| 96 | 05D04 | VL-CDR3 | QQYGNSPLT |
| 97 | 05D04 | VH | QVQLLESGPGLVKPSETLSLTCTVSNGSIRSYYWSWIRQPPGKGLEWIG |
| | | | YIYYSGSANYNPSLKSRVTISVDTSETQFSLKLSSVTAADTAVYYCASD |
| | | | RGPWGGGFDYWGQGTLVTVSS |
| 98 | 05D04 | VL | EIVLTQSPGTLSLSPGERATLSCRASQSVSSSYLAWYQQKPGQAPRLLI |
| | | | YGASSRATGIPDRFSGSGSGTDFTLTISRLEPEDFAVYYCQQYGNSPLT |
| | | | FGGGTKVEIK |
| 101 | 06C21 | VH-CDR1 | SYYWS |
| 102 | 06C21 | VH-CDR2 | YIYYNGNTNYNPSLKS |
| 103 | 06C21 | VH-CDR3 | DSGPWVPLDY |
| 104 | 06C21 | VL-CDR1 | RASQSVSSSYLA |
| 105 | 06C21 | VL-CDR2 | GASSRAT |
| 106 | 06C21 | VL-CDR3 | QQYGSSPPT |
| 107 | 06C21 | VH | QVQLQESGPGLVQPSETLSLTCTVSGSSISSYYWSWIRQPPGKGLEWIG |
| | | | YIYYNGNTNYNPSLKSRVTLSVDTSKNQFSLKVRSVTAADTAVYYCARD |
| | | | SGPWVPLDYWGQGTLVTVSS |
| 108 | 06C21 | VL | EIVLTQSPGTLSLSPGERATLSCRASQSVSSSYLAWYQQKPGQAPRLLI |
| | | | YGASSRATGIPDRFSGSGSGTDFTLTISRLEPEDFAVYYCQQYGSSPPT |
| | | | FGQGTKVEIK |
| 111 | 06M03 | VH-CDR1 | TYDMH |
| 112 | 06M03 | VH-CDR2 | TIGTAGDTKYSGSVKG |
| 113 | 06M03 | VH-CDR3 | TRAPVGAISDY |
| 114 | 06M03 | VL-CDR1 | TRSSGSIASNYVQ |
| 115 | 06M03 | VL-CDR2 | EDNQRPS |
| 116 | 06M03 | VL-CDR3 | SYDSINHVVF |
| 117 | 06M03 | VH | EVQLVESGGGLVQPGGSLRLSCAASGFTFSTYDMHWVRQVTGKGLEWVS |
| | | | TIGTAGDTKYSGSVKGRFTISRENAKNSLYLQMHSLRVGDTAVYYCTRA |
| | | | PVGAISDYWGQGTLVTVSS |
| 118 | 06M03 | VL | NFMLTQPHSVSESPGKTVTISCTRSSGSIASNYVQWYQQRPGNSPTIVI |
| | | | YEDNQRPSGVPDRFSGSIDSSSNSASLTISGLKTEDEADYYCQSYDSIN |
| | | | HVVFGGGTKLTVL |
| 121 | 07A15 | VH-CDR1 | SGGYY |
| 122 | 07A15 | VH-CDR2 | IGYIYNSGSTYYNPSLK |
| 123 | 07A15 | VH-CDR3 | DNGIVGATGGMDV |
| 124 | 07A15 | VL-CDR1 | RASQSVSSSYLA |
| 125 | 07A15 | VL-CDR2 | GASSRAT |
| 126 | 07A15 | VL-CDR3 | QQYGSSPLT |
| 127 | 07A15 | VH | QVQLQESGPGLVKPSQTLSLTCTVSGGSISSGGYYWSWIRQHPGKGLEW |
| | | | IGYIYNSGSTYYNPSLKSRVTISVDTSKNQFSLKLSSVTAADTAVYFCA |
| | | | GDNGIVGATGGMDVWGQGTTVTVSS |
| 128 | 07A15 | VL | EIVLTQSPGTLSLSPGERATLSCRASQSVSSSYLAWYQQKPGQAPRLLI |
| | | | YGASSRATGIPDRFSGSGSGTDFTLTISRLEPEDFAVYYCQQYGSSPLT |
| | | | FGGGTKVEIK |
| 131 | 07C10 | VH-CDR1 | SYYWS |
| 132 | 07C10 | VH-CDR2 | YIYYIGSTNYNPSLKS |
| 133 | 07C10 | VH-CDR3 | DRMGALDY |
| 134 | 07C10 | VL-CDR1 | RSSQSLVHSDGNTYLT |
| 135 | 07C10 | VL-CDR2 | EISNRFS |
| 136 | 07C10 | VL-CDR3 | MQAAQFPT |
| 137 | 07C10 | VH | QVQLQESGPGLVKPSETLSLTCTVSGGSINSYYWSWIRQPPGKGLEWIG |
| | | | YIYYIGSTNYNPSLKSRVTISMDTSKNQFSLKLSSVTAADTAVYYCARD |
| | | | RMGALDYWGQGTLVTVSS |
| 138 | 07C10 | VL | DIVMTQTPLSSPVTLGQPASISCRSSQSLVHSDGNTYLTWLQQRPGQPP |
| | | | RLLIYEISNRFSGVPDRFSGSGAGTDFTLKISRVEVEDVGVYYCMQAAQ |
| | | | FPTFGQGTKLEIK |
| 141 | 07E14 | VH-CDR1 | YYYWS |
| 142 | 07E14 | VH-CDR2 | YIYHSGSTNYNPSLKS |
| 143 | 07E14 | VH-CDR3 | ARDGGIVGATGGFDY |
| 144 | 07E14 | VL-CDR1 | RASQSVSSSYLA |
| 145 | 07E14 | VL-CDR2 | GASSRAT |
| 146 | 07E14 | VL-CDR3 | QQYGSSPIT |
| 147 | 07E14 | VH | QVQLQESGPGLVKPSETLSLTCTVSGGSISYYYWSWIRQPPGKGLEWIG |
| | | | YIYHSGSTNYNPSLKSRVTISIDTSENQFSLKLTSVTAADTAVYYCARD |
| | | | GGIVGATGGFDYWGQGTLVTVSS |
| 148 | 07E14 | VL | EIVLTQSPGTLSLSPGERATLSCRASQSVSSSYLAWYQQKPGQAPRLLI |
| | | | YGASSRATGIPDRFSGSGSGTDFTLTISRLEPEDFAVYYCQQYGSSPIT |
| | | | FGQGTRLEIK |
| 151 | 7H10 | VH-CDR1 | SYYWS |
| 152 | 7H10 | VH-CDR2 | RIYTSGSTNKNPSLKS |
| 153 | 7H10 | VH-CDR3 | DPLGSFFDY |
| 154 | 7H10 | VL-CDR1 | TGTSSDVGGYNYVS |
| 155 | 7H10 | VL-CDR2 | EVSERPS |
| 156 | 7H10 | VL-CDR3 | SSYAGSNNVV |
| 157 | 07H10 | VH | QVQLQESGPGLVKPSETLSLTCTVSGGSISSYYWSWIRQPAGKGLEWIG |
| | | | RIYTSGSTNKNPSLKSRITMSEDRSKNQFSLKLSSVTAADTAVYYCARD |
| | | | PLGSFFDYWGQGTLVIVSS |
| 158 | 07H10 | VL | QSALTQPPSASGSPGQSVTISCTGTSSDVGGYNYVSWYQQHPGKAPKFM |
| | | | IYEVSERPSGVPDRFSGSKSGNTASLTVSGLQAEDEADYYCSSYAGSNN |
| | | | VVFGGGTKLTVL |
| 161 | 07P15 | VH-CDR1 | SYYWS |
| 162 | 07P15 | VH-CDR2 | RIYTIGTTNYNPSLKS |
| 163 | 07P15 | VH-CDR3 | DPLGSFFDY |
| 164 | 07P15 | VL-CDR1 | TGTSSDVGGYNYVS |
| 165 | 07P15 | VL-CDR2 | EVSKRPS |
| 166 | 07P15 | VL-CDR3 | SSYAGINNLV |
| 167 | 07P15 | VH | QVQLQESGPGLVKPSETLSLTCTVSGGSISSYYWSWIRQPAGKGLEWIG |
| | | | RIYTIGTTNYNPSLKSRVTMSVDTSKNQFSLKLSSVTAADAAVYYCARD |
| | | | PLGSFFDYWGQGSLVTVSS |
| 168 | 07P15 | VL | QSALTQPPSASGSPGQSVTISCTGTSSDVGGYNYVSWYQQHPGKAPKLM |
| | | | IYEVSKRPSGVPDRFSGSKSGNTASLTVSGLQAEDEADYYCSSYAGINN |
| | | | LVFGGGTKLTVL |
| 171 | 08H12 | VH-CDR1 | SYYWS |
| 172 | 08H12 | VH-CDR2 | YIYYIGSTNYNPSLKS |
| 173 | 08H12 | VH-CDR3 | DMMGALDY |
| 174 | 08H12 | VL-CDR1 | RSSESLVHSDGNTYLS |
| 175 | 08H12 | VL-CDR2 | KISNRFS |
| 176 | 08H12 | VL-CDR3 | MQAAQFPT |
| 177 | 08H12 | VH | QVQLQESGPGLVKPSETLSLTCTVSGGSISSYYWSWIRQPPGRGLEWIG |
| | | | YIYYIGSTNYNPSLKSRVTISLDTSKNQFSLKLNSVTAADTAVYYCARD |
| | | | MMGALDYWGQGTLVTVSS |
| 178 | 08H12 | VL | DIVMTQTPLSSPVTLGQPASISCRSSESLVHSDGNTYLSWLQQRPGQPP |
| | | | RLLIYKISNRFSGVPDRFSGSGAGTDFTLKISRVEAEDVGVYYCMQAAQ |
| | | | FPTFGQGTRLEIK |
| 181 | 08L01 | VH-CDR1 | SYYWS |
| 182 | 08L01 | VH-CDR2 | RIYTSGSTNFKPSLKS |
| 183 | 08L01 | VH-CDR3 | DPLGSFFDY |
| 184 | 08L01 | VL-CDR1 | TGTSSDVGGYNYVS |
| 185 | 08L01 | VL-CDR2 | EVSKRPS |
| 186 | 08L01 | VL-CDR3 | SSYADINNLV |
| 187 | 08L01 | VH | QVQLQESGPGLVKPSETLSLTCTVSGGSISSYYWSWIRQPAGKGLEWIG |
| | | | RIYTSGSTNFKPSLKSRVTMSVDTSKNQFSLKLRSMTAADTAVYYCARD |
| | | | PLGSFFDYWGQGTLVTVSS |
| 188 | 08L01 | VL | QSALTQPPSASGSPGQSVTISCTGTSSDVGGYNYVSWYQQHPGKAPKLM |
| | | | IYEVSKRPSGVPDRFSGSKSGNTASLTVSGLQAEDEADYYCSSYADINN |
| | | | LVFGTGTKVTVL |
| 191 | 08N13 | VH-CDR1 | GYYMH |
| 192 | 08N13 | VH-CDR2 | WINPNSGGTNYAQKFQG |
| 193 | 08N13 | VH-CDR3 | GAWGSN |
| 194 | 08N13 | VL-CDR1 | RASQGISNYLA |
| 195 | 08N13 | VL-CDR2 | AASSLQS |
| 196 | 08N13 | VL-CDR3 | QQYNSYPYT |
| 197 | 08N13 | VH | QVQLVQSGAEVKKPGASVKVSCKASGYTFTGYYMHWVRQAPGQGLEWMG |
| | | | WINPNSGGTNYAQKFQGRVTMTRDTSINTAYMELSRLRSDDTAVYYCAR |
| | | | GAWGSNWGQGTLVTVSS |
| 198 | 08N13 | VL | DIQMTQSPSSLSASVGDRVTITCRASQGISNYLAWFQQKPGKAPKSLIY |
| | | | AASSLQSGVPSKFSGSGSGTDFTLTISSLQPEDFATYYCQQYNSYPYTF |
| | | | GQGTKLEIK |
| 201 | 09H07 | VH-CDR1 | YFYWT |
| 202 | 09H07 | VH-CDR2 | YIYHSGSTNYNPSLKS |
| 203 | 09H07 | VH-CDR3 | DGGIVGATNALDI |
| 204 | 09H07 | VL-CDR1 | RASQSVSSSYLA |
| 205 | 09H07 | VL-CDR2 | GASSRAT |
| 206 | 09H07 | VL-CDR3 | QQYGSSPLT |
| 207 | 09H07 | VH | QVQLQESGPGLVKPSETLSLTCTVSGGSIDYFYWTWIRQSPGKGLEWIG |
| | | | YIYHSGSTNYNPSLKSRVTMSLDTSKNQFSLKLTSVTTADTAVYYCARD |
| | | | GGIVGATNALDIWGQGTMVTVSS |
| 208 | 09H07 | VL | EIVLTQSPGTLSLSPGERATLSCRASQSVSSSYLAWYQQKPGQAPRLLI |
| | | | YGASSRATGIPDRFSGSGSGTDFTLTISRLEPEDFAVYYCQQYGSSPLT |
| | | | FGGGTKVEIK |
| 211 | 09013 | VH-CDR1 | GYYWS |
| 212 | 09013 | VH-CDR2 | YIYYSVSSNYNPSLKS |
| 213 | 09013 | VH-CDR3 | ARDETGYFDY |
| 214 | 09013 | VL-CDR1 | RASQSVRSSYLA |
| 215 | 09013 | VL-CDR2 | GASSRAT |
| 216 | 09013 | VL-CDR3 | QQYGSSLT |
| 217 | 09013 | VH | QVQLQESGPGLVKPSETLSLTCTVSGGSISGYYWSWIRQTPGKGLEWIG |
| | | | YIYYSVSSNYNPSLKSRVTISVDTSKNQFSLRLSSVTAADTAVYYCARD |
| | | | ETGYFDYWGQGTLVTVSS |
| 218 | 09013 | VL | EIVLTQSPGTLSLSPGERATLSCRASQSVRSSYLAWYQQKPGQAPRLLI |
| | | | YGASSRATGIPDRFSGSGSGTDFTLTISRLEPEDFAVYYCQQYGSSLTF |
| | | | GGGTKVEIK |
| 221 | 10E03 | VH-CDR1 | SYYWS |
| 222 | 10E03 | VH-CDR2 | RLYTSGSTNYNPSLKS |
| 223 | 10E03 | VH-CDR3 | ARDPLGSFFDY |
| 224 | 10E03 | VL-CDR1 | TGTSSDVGGYNYVS |
| 225 | 10E03 | VL-CDR2 | EVSKRPS |
| 226 | 10E03 | VL-CDR3 | SSYAGSNNVV |
| 227 | 10E03 | VH | QVQLQESGPGLVKPSETLSLTCTVSGDSFSSYYWSWIRQPAGKGLEWIG |
| | | | RLYTSGSTNYNPSLKSRVTVSVDTSKNQFSLKLNSVTAADTAVYYCARD |
| | | | PLGSFFDYWGQGTLVTVSS |
| 228 | 10E03 | VL | QSALTQPPSASGSPGQSVTISCTGTSSDVGGYNYVSWYQQHPGKAPKLM |
| | | | IYEVSKRPSGVPDRFSGSKSGNTASLTVSGLQAEDEADYYCSSYAGSNN |
| | | | VVFGGGTKLTVL |
| 231 | 10F07 | VH-CDR1 | TSGVGVG |
| 232 | 10F07 | VH-CDR2 | LIYWDDDKLYSPSLKS |
| 233 | 10F07 | VH-CDR3 | LGMRHAFDI |
| 234 | 10F07 | VL-CDR1 | TGSSSNIGAGYDVH |
| 235 | 10F07 | VL-CDR2 | GNRYRPS |
| 236 | 10F07 | VL-CDR3 | QSYDSSLSVV |
| 237 | 10F07 | VH | QITLKESGPTLVKPTQTLTLTCTFSGFSLSTSGVGVGWIRQPPEKALEW |
| | | | LALIYWDDDKLYSPSLKSRLTITKDTSKNQVVLTLTNMDPVDTATYYCA |
| | | | RLGMRHAFDIWGQGTMVTVSS |
| 238 | 10F07 | VL | QSVLTQPPSVSGAPGQRVTISCTGSSSNIGAGYDVHWFQQLPGTAPKLL |
| | | | IYGNRYRPSGVPDRFSGSKSGTSASLAIAGLQAEDEADYYCQSYDSSLS |
| | | | VVFGGGTKLTVL |
| 241 | 10G03 | VH-CDR1 | GYYWS |
| 242 | 10G03 | VH-CDR2 | YVYYSVSTDYNPSLKS |
| 243 | 10G03 | VH-CDR3 | DETGYFDY |
| 244 | 10G03 | VL-CDR1 | RASQSVRSSYLA |
| 245 | 10G03 | VL-CDR2 | GASSRAT |
| 246 | 10G03 | VL-CDR3 | QQYGSSLT |
| 247 | 10G03 | VH | QVQLQESGPGLVKPSETLSLTCTVSGGSISGYYWSWIRQTPGKGLEWIG |
| | | | YVYYSVSTDYNPSLKSRVTISVDTSKNQFSLRLSSVTAADTAVYYCARD |
| | | | ETGYFDYWGQGTLVTVSS |
| 248 | 10G03 | VL | EIVLTQSPGTLSLSPGERATLSCRASQSVRSSYLAWYQQKPGQAPRLLI |
| | | | YGASSRATGIPDRFSGSGSGTDFTLTISRLEPEDFAVYYCQQYGSSLTF |
| | | | GGGTKVEIK |
| 251 | 10P02 | VH-CDR1 | TYYWS |
| 252 | 10P02 | VH-CDR2 | RIYTSGTTNYNPSLKS |
| 253 | 10P02 | VH-CDR3 | DPLGSFFDY |
| 254 | 10P02 | VL-CDR1 | TGTSSDVGGYNYVS |
| 255 | 10P02 | VL-CDR2 | EVSKRPS |
| 256 | 10P02 | VL-CDR3 | SSYAGINNVV |
| 257 | 10P02 | VH | QVQLQESGPGLVKPSETLSLTCTVSGDSIRTYYWSWIRQPAGKGLEWIG |
| | | | RIYTSGTTNYNPSLKSRVTMSEDTSNNQFSLKLSSVTAADTAVYYCARD |
| | | | PLGSFFDYWGQGTLVTVSS |
| 258 | 10P02 | VL | QSALTQPPSASGSPGQSVTISCTGTSSDVGGYNYVSWYQQHPGKAPKLM |
| | | | IYEVSKRPSGVPDRFSGSKSGNTASLTVSGLQAEDEADYYCSSYAGINN |
| | | | VVFGGGTKLTVL |
| 261 | 11H13 | VH-CDR1 | SYDMH |
| 262 | 11H13 | VH-CDR2 | AIGTAGDTYYPGSVKG |
| 263 | 11H13 | VH-CDR3 | DSGGLSYAFDI |
| 264 | 11H13 | VL-CDR1 | RASQGINNYLA |
| 265 | 11H13 | VL-CDR2 | AASSLLS |
| 266 | 11H13 | VL-CDR3 | LQHNSYPPT |
| 267 | 11H13 | VH | EVQLVESGGGLVQPGGSLRLSCAASGFTFSSYDMHWVRQATGKGLEWVS |
| | | | AIGTAGDTYYPGSVKGRFTISRENAKSSLYLQMNSLRAGDTAVYYCARD |
| | | | SGGLSYAFDIWGQGTMVTVSS |
| 268 | 11H13 | VL | DIQMTQSPSAMSASVGDRVAITCRASQGINNYLAWFQQKPGKVPKRLIY |
| | | | AASSLLSGVPSRFSGSGSGTEFTLTISSLQPEDFATYYCLQHNSYPPTF |
| | | | GQGTKVEIK |
| 271 | 12G06 | VH-CDR1 | SNYWT |
| 272 | 12G06 | VH-CDR2 | YIYYIGSTNYNPSLKS |
| 273 | 12G06 | VH-CDR3 | ARDGGYSGSYWGLDP |
| 274 | 12G06 | VL-CDR1 | RASQSVSSRSLA |
| 275 | 12G06 | VL-CDR2 | GASSRAT |
| 276 | 12G06 | VL-CDR3 | QQYVTSPIT |
| 277 | 12G06 | VH | QVQLQESGPGLVKPSETLSLTCTVSGGSINSNYWTWIRQPPGKGLEWIG |
| | | | YIYYIGSTNYNPSLKSRVTISIDTSKNHFSLKLSSVTAADTAVYYCARD |
| | | | GGYSGSYWGLDPWGQGTLVTVSS |
| 278 | 12G06 | VL | EIVLTQSPGTLSLSPGERATLSCRASQSVSSRSLAWYQQKPGQAPRLLI |
| | | | YGASSRATGIPDRFSGSGSGTDFTLTISRLEPEDFAVYYCQQYVTSPIT |
| | | | FGQGTRLEIK |
| 281 | 12H24 | VH-CDR1 | YYYWS |
| 282 | 12H24 | VH-CDR2 | YIYFSGSTNYNPSLKS |
| 283 | 12H24 | VH-CDR3 | ARDGGIVGATEAFDI |
| 284 | 12H24 | VL-CDR1 | RASQSISGSYLA |
| 285 | 12H24 | VL-CDR2 | GASSRAT |
| 286 | 12H24 | VL-CDR3 | QQYGSSPIT |
| 287 | 12H24 | VH | QVQLQESGPGLVKPSETLSLTCTVSGGSISYYYWSWIRQPPGKGLEWIG |
| | | | YIYFSGSTNYNPSLKSRVTMSVDTSKNQFSLKLTSVTAADTAVYYCARD |
| | | | GGIVGATEAFDIWGQGTMVTVSS |
| 288 | 12H24 | VL | EIVLTQSPGTLSLSPGERATLSCRASQSISGSYLAWYQQKPGQAPRLLI |
| | | | YGASSRATGIPDRFSGSGSGTDFTLTISRLEPEDFAVYYCQQYGSSPIT |
| | | | FGQGTRLEIK |
| 291 | 12M06 | VH-CDR1 | SYYWT |
| 292 | 12M06 | VH-CDR2 | YIYYNGNTNYNPSLKS |
| 293 | 12M06 | VH-CDR3 | ARDGGPWVPLDY |
| 294 | 12M06 | VL-CDR1 | RASQSVSSSYLA |
| 295 | 12M06 | VL-CDR2 | GASSRAT |
| 296 | 12M06 | VL-CDR3 | QQYGSSPPT |
| 297 | 12M06 | VH | QVQLQESGPGLVKPSETLSLTCTVSGGSISSYYWTWIRQPPGKGLEWIG |
| | | | YIYYNGNTNYNPSLKSRVTMSVETSKNQFSLKLRSVTAADTAVYYCARD |
| | | | GGPWVPLDYWGQGTLVTVSS |
| 298 | 12M06 | VL | EIVLTQSPGTLSLSPGERATLSCRASQSVSSSYLAWYQQKPGQAPRLLI |
| | | | YGASSRATGIPDRFSGSGSGTDFTLTISRLEPEAFAVYYCQQYGSSPPT |
| | | | FGQGTKVEIK |
| 301 | 13M02 | VH-CDR1 | FYYWS |
| 302 | 13M02 | VH-CDR2 | RIYTSGSTNYNPSLKS |
| 303 | 13M02 | VH-CDR3 | ARDPLGTFFDY |
| 304 | 13M02 | VL-CDR1 | TGTSSDVGGYNYVS |
| 305 | 13M02 | VL-CDR2 | EVSKRPS |
| 306 | 13M02 | VL-CDR3 | SSYAGINNVV |
| 307 | 13M02 | VH | QVQLQESGPGLVKPSETLSLTCTVSGSSISFYYWSWIRQPAGKGLEWIG |
| | | | RIYTSGSTNYNPSLKSRLTMSLDTSKNQFSLRLSSVTAADTAVYYCARD |
| | | | PLGTFFDYWGQGTLVTVSS |
| 308 | 13M02 | VL | QSALTQPPSASGSPGQSVTISCTGTSSDVGGYNYVSWYQQHPGKAPKLM |
| | | | IYEVSKRPSGVPDRFSGSKSGNTASLTVSGLQAEDEADYYCSSYAGINN |
| | | | VVFGGGTKLTVL |
| 311 | 13P04 | VH-CDR1 | SYYWS |
| 312 | 13P04 | VH-CDR2 | RIYTIGTTNYNPSLKS |
| 313 | 13P04 | VH-CDR3 | DPLGTFFDY |
| 314 | 13P04 | VL-CDR1 | TGTSSDVGGHNYVS |
| 315 | 13P04 | VL-CDR2 | EVTKRPS |
| 316 | 13P04 | VL-CDR3 | SSYAGINNLV |
| 317 | 13P04 | VH | QVQLQESGPGLVKPSETLSLTCTVSGDSISSYYWSWIRQPAGKGLEWIG |
| | | | RIYTIGTTNYNPSLKSRVTMSVDTSKNQFSLKLNSVTAADAAVYYCARD |
| | | | PLGTFFDYWGQGFLVTVSS |
| 318 | 13P04 | VL | QSALTQPPSASGSPGQSVTISCTGTSSDVGGHNYVSWYQQHPGKAPKFM |
| | | | IYEVTKRPSGVPDRFSGSKSGNTASLTVSGLQAEDEADYYCSSYAGINN |
| | | | LVFGGGTKLTVL |
| 321 | 14A24 | VH-CDR1 | FYYWT |
| 322 | 14A24 | VH-CDR2 | YIYHSGSTNYNPSLKS |
| 323 | 14A24 | VH-CDR3 | DGGIVGATNAFDI |
| 324 | 14A24 | VL-CDR1 | RASQSVTSSYLA |
| 325 | 14A24 | VL-CDR2 | GASSRAT |
| 326 | 14A24 | VL-CDR3 | QQYGSSPLT |
| 327 | 14A24 | VH | QVQLQESGPGLVKPSETLSLSCTVSGGSISFYYWTWIRQPPGKGLEWIG |
| | | | YIYHSGSTNYNPSLKSRVTISVDTSKNQFSLKLSSVTAADTAVYYCARD |
| | | | GGIVGATNAFDIWGQGTMVTVSS |
| 328 | 14A24 | VL | EIVLTQSPGTLSLSPGERATLSCRASQSVTSSYLAWYQQKPGQAPRLLI |
| | | | YGASSRATGIPDRFSGSGSGTDFTLTISRLEPEDFAVYYCQQYGSSPLT |
| | | | FGGGTKVEIK |
| 331 | 14M02 | VH-CDR1 | SYYWS |
| 332 | 14M02 | VH-CDR2 | YIYYIGTTNYNPSLKS |
| 333 | 14M02 | VH-CDR3 | DLGIEGGFDY |
| 334 | 14M02 | VL-CDR1 | RASQSVSSSLA |
| 335 | 14M02 | VL-CDR2 | GASTRAT |
| 336 | 14M02 | VL-CDR3 | QQYNNWPLT |
| 337 | 14M02 | VH | QVQLQESGPGLVKPSETLSLTCTVSAVSINSYYWSWIRQPPGKGLEWIG |
| | | | YIYYIGTTNYNPSLKSRVTILLDTSKNQFSLKLSSVTAADTAVYYCARD |
| | | | LGIEGGFDYWGQGTLVTVSS |
| 338 | 14M02 | VL | EIVMTQSPASLSVSPGERATLSCRASQSVSSSLAWYQQKVGQAPRLLIS |
| | | | GASTRATGIPARFSGSGSGTEFTLTISSLQSEDFAVYYCQQYNNWPLTF |
| | | | GGGTKVEIK |

In some aspects, the antibody or antigen-binding portion thereof comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antibody or antigen-binding portion thereof cross-competes for binding to human CD161 with a reference antibody, wherein the reference antibody comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antibody or antigen-binding portion thereof binds the same epitope on human CD161 as a reference antibody, wherein the reference antibody comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antibody or antigen-binding portion thereof comprises a VH-CDR1 comprising the amino acid sequence set forth in SEQ ID NO: 91, a VH-CDR2 comprising the amino acid sequence set forth in SEQ ID NO: 92, a VH-CDR3 comprising the amino acid sequence set forth in SEQ ID NO: 93, a VL-CDR1 comprising the amino acid sequence set forth in SEQ ID NO: 94, a VL-CDR2 comprising the amino acid sequence set forth in SEQ ID NO: 95, and a VL-CDR3 comprising the amino acid sequence set forth in SEQ ID NO: 96.

In some aspects, the antibody or antigen-binding portion thereof cross-competes for binding to human CD161 with a reference antibody, wherein the reference antibody comprises a VH-CDR1 comprising the amino acid sequence set forth in SEQ ID NO: 91, a VH-CDR2 comprising the amino acid sequence set forth in SEQ ID NO: 92, a VH-CDR3 comprising the amino acid sequence set forth in SEQ ID NO: 93, a VL-CDR1 comprising the amino acid sequence set forth in SEQ ID NO: 94, a VL-CDR2 comprising the amino acid sequence set forth in SEQ ID NO: 95, and a VL-CDR3 comprising the amino acid sequence set forth in SEQ ID NO: 96.

In some aspects, the antibody or antigen-binding portion thereof binds the same epitope on human CD161 as a reference antibody, wherein the reference antibody comprises a VH-CDR1 comprising the amino acid sequence set forth in SEQ ID NO: 91, a VH-CDR2 comprising the amino acid sequence set forth in SEQ ID NO: 92, a VH-CDR3 comprising the amino acid sequence set forth in SEQ ID NO: 93, a VL-CDR1 comprising the amino acid sequence set forth in SEQ ID NO: 94, a VL-CDR2 comprising the amino acid sequence set forth in SEQ ID NO: 95, and a VL-CDR3 comprising the amino acid sequence set forth in SEQ ID NO: 96.

In some aspects, the antibody or antigen-binding portion thereof comprises a VH-CDR1 comprising the amino acid sequence set forth in SEQ ID NO: 111, a VH-CDR2 comprising the amino acid sequence set forth in SEQ ID NO: 112, a VH-CDR3 comprising the amino acid sequence set forth in SEQ ID NO: 113, a VL-CDR1 comprising the amino acid sequence set forth in SEQ ID NO: 114, a VL-CDR2 comprising the amino acid sequence set forth in SEQ ID NO: 115, and a VL-CDR3 comprising the amino acid sequence set forth in SEQ ID NO: 116.

In some aspects, the antibody or antigen-binding portion thereof cross-competes for binding to human CD161 with a reference antibody, wherein the reference antibody comprises a VH-CDR1 comprising the amino acid sequence set forth in SEQ ID NO: 111, a VH-CDR2 comprising the amino acid sequence set forth in SEQ ID NO: 112, a VH-CDR3 comprising the amino acid sequence set forth in SEQ ID NO: 113, a VL-CDR1 comprising the amino acid sequence set forth in SEQ ID NO: 114, a VL-CDR2 comprising the amino acid sequence set forth in SEQ ID NO: 115, and a VL-CDR3 comprising the amino acid sequence set forth in SEQ ID NO: 116.

In some aspects, the antibody or antigen-binding portion thereof binds the same epitope on human CD161 as a reference antibody, wherein the reference antibody comprises a VH-CDR1 comprising the amino acid sequence set forth in SEQ ID NO: 111, a VH-CDR2 comprising the amino acid sequence set forth in SEQ ID NO: 112, a VH-CDR3 comprising the amino acid sequence set forth in SEQ ID NO: 113, a VL-CDR1 comprising the amino acid sequence set forth in SEQ ID NO: 114, a VL-CDR2 comprising the amino acid sequence set forth in SEQ ID NO: 115, and a VL-CDR3 comprising the amino acid sequence set forth in SEQ ID NO: 116.

In some aspects, the antibody or antigen-binding portion thereof comprises a VH-CDR1 comprising the amino acid sequence set forth in SEQ ID NO: 141, a VH-CDR2 comprising the amino acid sequence set forth in SEQ ID NO: 142, a VH-CDR3 comprising the amino acid sequence set forth in SEQ ID NO: 143, a VL-CDR1 comprising the amino acid sequence set forth in SEQ ID NO: 144, a VL-CDR2 comprising the amino acid sequence set forth in SEQ ID NO: 145, and a VL-CDR3 comprising the amino acid sequence set forth in SEQ ID NO: 146.

In some aspects, the antibody or antigen-binding portion thereof cross-competes for binding to human CD161 with a reference antibody, wherein the reference antibody comprises a VH-CDR1 comprising the amino acid sequence set forth in SEQ ID NO: 141, a VH-CDR2 comprising the amino acid sequence set forth in SEQ ID NO: 142, a VH-CDR3 comprising the amino acid sequence set forth in SEQ ID NO: 143, a VL-CDR1 comprising the amino acid sequence set forth in SEQ ID NO: 144, a VL-CDR2 comprising the amino acid sequence set forth in SEQ ID NO: 145, and a VL-CDR3 comprising the amino acid sequence set forth in SEQ ID NO: 146.

In some aspects, the antibody or antigen-binding portion thereof binds the same epitope on human CD161 as a reference antibody, wherein the reference antibody comprises a VH-CDR1 comprising the amino acid sequence set forth in SEQ ID NO: 141, a VH-CDR2 comprising the amino acid sequence set forth in SEQ ID NO: 142, a VH-CDR3 comprising the amino acid sequence set forth in SEQ ID NO: 143, a VL-CDR1 comprising the amino acid sequence set forth in SEQ ID NO: 144, a VL-CDR2 comprising the amino acid sequence set forth in SEQ ID NO: 145, and a VL-CDR3 comprising the amino acid sequence set forth in SEQ ID NO: 146.

In some aspects, the antibody or antigen-binding portion thereof comprises a VH-CDR1 comprising the amino acid sequence set forth in SEQ ID NO: 211, a VH-CDR2 comprising the amino acid sequence set forth in SEQ ID NO: 212, a VH-CDR3 comprising the amino acid sequence set forth in SEQ ID NO: 213, a VL-CDR1 comprising the amino acid sequence set forth in SEQ ID NO: 214, a VL-CDR2 comprising the amino acid sequence set forth in SEQ ID NO: 215, and a VL-CDR3 comprising the amino acid sequence set forth in SEQ ID NO: 216.

In some aspects, the antibody or antigen-binding portion thereof cross-competes for binding to human CD161 with a reference antibody, wherein the reference antibody comprises a VH-CDR1 comprising the amino acid sequence set forth in SEQ ID NO: 211, a VH-CDR2 comprising the amino acid sequence set forth in SEQ ID NO: 212, a VH-CDR3 comprising the amino acid sequence set forth in SEQ ID NO: 213, a VL-CDR1 comprising the amino acid sequence set forth in SEQ ID NO: 214, a VL-CDR2 comprising the amino acid sequence set forth in SEQ ID NO: 215, and a VL-CDR3 comprising the amino acid sequence set forth in SEQ ID NO: 216.

In some aspects, the antibody or antigen-binding portion thereof binds the same epitope on human CD161 as a reference antibody, wherein the reference antibody comprises a VH-CDR1 comprising the amino acid sequence set forth in SEQ ID NO: 211, a VH-CDR2 comprising the amino acid sequence set forth in SEQ ID NO: 212, a VH-CDR3 comprising the amino acid sequence set forth in SEQ ID NO: 213, a VL-CDR1 comprising the amino acid sequence set forth in SEQ ID NO: 214, a VL-CDR2 comprising the amino acid sequence set forth in SEQ ID NO: 215, and a VL-CDR3 comprising the amino acid sequence set forth in SEQ ID NO: 216.

In some aspects, the antibody or antigen-binding portion thereof comprises a VH-CDR1 comprising the amino acid sequence set forth in SEQ ID NO: 221, a VH-CDR2 comprising the amino acid sequence set forth in SEQ ID NO: 222, a VH-CDR3 comprising the amino acid sequence set forth in SEQ ID NO: 223, a VL-CDR1 comprising the amino acid sequence set forth in SEQ ID NO: 224, a VL-CDR2 comprising the amino acid sequence set forth in SEQ ID NO: 225, and a VL-CDR3 comprising the amino acid sequence set forth in SEQ ID NO: 226.

In some aspects, the antibody or antigen-binding portion thereof cross-competes for binding to human CD161 with a reference antibody, wherein the reference antibody comprises a VH-CDR1 comprising the amino acid sequence set forth in SEQ ID NO: 221, a VH-CDR2 comprising the amino acid sequence set forth in SEQ ID NO: 222, a VH-CDR3 comprising the amino acid sequence set forth in SEQ ID NO: 223, a VL-CDR1 comprising the amino acid sequence set forth in SEQ ID NO: 224, a VL-CDR2 comprising the amino acid sequence set forth in SEQ ID NO: 225, and a VL-CDR3 comprising the amino acid sequence set forth in SEQ ID NO: 226.

In some aspects, the antibody or antigen-binding portion thereof binds the same epitope on human CD161 as a reference antibody, wherein the reference antibody comprises a VH-CDR1 comprising the amino acid sequence set forth in SEQ ID NO: 221, a VH-CDR2 comprising the amino acid sequence set forth in SEQ ID NO: 222, a VH-CDR3 comprising the amino acid sequence set forth in SEQ ID NO: 223, a VL-CDR1 comprising the amino acid sequence set forth in SEQ ID NO: 224, a VL-CDR2 comprising the amino acid sequence set forth in SEQ ID NO: 225, and a VL-CDR3 comprising the amino acid sequence set forth in SEQ ID NO: 226.

In some aspects, the antibody or antigen-binding portion thereof comprises a VH-CDR1 comprising the amino acid sequence set forth in SEQ ID NO: 271, a VH-CDR2 comprising the amino acid sequence set forth in SEQ ID NO: 272, a VH-CDR3 comprising the amino acid sequence set forth in SEQ ID NO: 273, a VL-CDR1 comprising the amino acid sequence set forth in SEQ ID NO: 274, a VL-CDR2 comprising the amino acid sequence set forth in SEQ ID NO: 275, and a VL-CDR3 comprising the amino acid sequence set forth in SEQ ID NO: 276.

In some aspects, the antibody or antigen-binding portion thereof cross-competes for binding to human CD161 with a reference antibody, wherein the reference antibody comprises a VH-CDR1 comprising the amino acid sequence set forth in SEQ ID NO: 271, a VH-CDR2 comprising the amino acid sequence set forth in SEQ ID NO: 272, a VH-CDR3 comprising the amino acid sequence set forth in SEQ ID NO: 273, a VL-CDR1 comprising the amino acid sequence set forth in SEQ ID NO: 274, a VL-CDR2 comprising the amino acid sequence set forth in SEQ ID NO: 275, and a VL-CDR3 comprising the amino acid sequence set forth in SEQ ID NO: 276.

In some aspects, the antibody or antigen-binding portion thereof binds the same epitope on human CD161 as a reference antibody, wherein the reference antibody comprises a VH-CDR1 comprising the amino acid sequence set forth in SEQ ID NO: 271, a VH-CDR2 comprising the amino acid sequence set forth in SEQ ID NO: 272, a VH-CDR3 comprising the amino acid sequence set forth in SEQ ID NO: 273, a VL-CDR1 comprising the amino acid sequence set forth in SEQ ID NO: 274, a VL-CDR2 comprising the amino acid sequence set forth in SEQ ID NO: 275, and a VL-CDR3 comprising the amino acid sequence set forth in SEQ ID NO: 276.

In some aspects, the antibody or antigen-binding portion thereof comprises a VH-CDR1 comprising the amino acid sequence set forth in SEQ ID NO: 281, a VH-CDR2 comprising the amino acid sequence set forth in SEQ ID NO: 282, a VH-CDR3 comprising the amino acid sequence set forth in SEQ ID NO: 283, a VL-CDR1 comprising the amino acid sequence set forth in SEQ ID NO: 284, a VL-CDR2 comprising the amino acid sequence set forth in SEQ ID NO: 285, and a VL-CDR3 comprising the amino acid sequence set forth in SEQ ID NO: 286.

In some aspects, the antibody or antigen-binding portion thereof cross-competes for binding to human CD161 with a reference antibody, wherein the reference antibody comprises a VH-CDR1 comprising the amino acid sequence set forth in SEQ ID NO: 281, a VH-CDR2 comprising the amino acid sequence set forth in SEQ ID NO: 282, a VH-CDR3 comprising the amino acid sequence set forth in SEQ ID NO: 283, a VL-CDR1 comprising the amino acid sequence set forth in SEQ ID NO: 284, a VL-CDR2 comprising the amino acid sequence set forth in SEQ ID NO: 285, and a VL-CDR3 comprising the amino acid sequence set forth in SEQ ID NO: 286.

In some aspects, the antibody or antigen-binding portion thereof binds the same epitope on human CD161 as a reference antibody, wherein the reference antibody comprises a VH-CDR1 comprising the amino acid sequence set forth in SEQ ID NO: 281, a VH-CDR2 comprising the amino acid sequence set forth in SEQ ID NO: 282, a VH-CDR3 comprising the amino acid sequence set forth in SEQ ID NO: 283, a VL-CDR1 comprising the amino acid sequence set forth in SEQ ID NO: 284, a VL-CDR2 comprising the amino acid sequence set forth in SEQ ID NO: 285, and a VL-CDR3 comprising the amino acid sequence set forth in SEQ ID NO: 286.

In some aspects, the antibody or antigen-binding portion thereof comprises a VH-CDR1 comprising the amino acid sequence set forth in SEQ ID NO: 291, a VH-CDR2 comprising the amino acid sequence set forth in SEQ ID NO: 292, a VH-CDR3 comprising the amino acid sequence set forth in SEQ ID NO: 293, a VL-CDR1 comprising the amino acid sequence set forth in SEQ ID NO: 294, a VL-CDR2 comprising the amino acid sequence set forth in SEQ ID NO: 295, and a VL-CDR3 comprising the amino acid sequence set forth in SEQ ID NO: 296.

In some aspects, the antibody or antigen-binding portion thereof cross-competes for binding to human CD161 with a reference antibody, wherein the reference antibody comprises a VH-CDR1 comprising the amino acid sequence set forth in SEQ ID NO: 291, a VH-CDR2 comprising the amino acid sequence set forth in SEQ ID NO: 292, a VH-CDR3 comprising the amino acid sequence set forth in SEQ ID NO: 293, a VL-CDR1 comprising the amino acid sequence set forth in SEQ ID NO: 294, a VL-CDR2 comprising the amino acid sequence set forth in SEQ ID NO: 295, and a VL-CDR3 comprising the amino acid sequence set forth in SEQ ID NO: 296.

In some aspects, the antibody or antigen-binding portion thereof binds the same epitope on human CD161 as a reference antibody, wherein the reference antibody comprises a VH-CDR1 comprising the amino acid sequence set forth in SEQ ID NO: 291, a VH-CDR2 comprising the amino acid sequence set forth in SEQ ID NO: 292, a VH-CDR3 comprising the amino acid sequence set forth in SEQ ID NO: 293, a VL-CDR1 comprising the amino acid sequence set forth in SEQ ID NO: 294, a VL-CDR2 comprising the amino acid sequence set forth in SEQ ID NO: 295, and a VL-CDR3 comprising the amino acid sequence set forth in SEQ ID NO: 296.

In some aspects, the antibody or antigen-binding portion thereof comprises a VH-CDR1 comprising the amino acid sequence set forth in SEQ ID NO: 301, a VH-CDR2 comprising the amino acid sequence set forth in SEQ ID NO: 302, a VH-CDR3 comprising the amino acid sequence set forth in SEQ ID NO: 303, a VL-CDR1 comprising the amino acid sequence set forth in SEQ ID NO: 304, a VL-CDR2 comprising the amino acid sequence set forth in SEQ ID NO: 305, and a VL-CDR3 comprising the amino acid sequence set forth in SEQ ID NO: 306.

In some aspects, the antibody or antigen-binding portion thereof cross-competes for binding to human CD161 with a reference antibody, wherein the reference antibody comprises a VH-CDR1 comprising the amino acid sequence set forth in SEQ ID NO: 301, a VH-CDR2 comprising the amino acid sequence set forth in SEQ ID NO: 302, a VH-CDR3 comprising the amino acid sequence set forth in SEQ ID NO: 303, a VL-CDR1 comprising the amino acid sequence set forth in SEQ ID NO: 304, a VL-CDR2 comprising the amino acid sequence set forth in SEQ ID NO: 305, and a VL-CDR3 comprising the amino acid sequence set forth in SEQ ID NO: 306.

In some aspects, the antibody or antigen-binding portion thereof binds the same epitope on human CD161 as a reference antibody, wherein the reference antibody comprises a VH-CDR1 comprising the amino acid sequence set forth in SEQ ID NO: 301, a VH-CDR2 comprising the amino acid sequence set forth in SEQ ID NO: 302, a VH-CDR3 comprising the amino acid sequence set forth in SEQ ID NO: 303, a VL-CDR1 comprising the amino acid sequence set forth in SEQ ID NO: 304, a VL-CDR2 comprising the amino acid sequence set forth in SEQ ID NO: 305, and a VL-CDR3 comprising the amino acid sequence set forth in SEQ ID NO: 306.

In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, and 337. In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 7. In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 97. In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 117. In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 147. In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 217. In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 227. In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 277. In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 287. In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 297. In some aspects, the antibody or antigen-binding portion thereof comprises a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 307.

In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, and 338. In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 98. In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 118. In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 148. In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 218. In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 228. In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 278. In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 288. In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 298. In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 308.

In some aspects, the antibody or antigen-binding portion thereof comprises a VH comprising an amino acid sequence selected from SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, and 337. In some aspects, the antibody or antigen-binding portion thereof comprises a VH comprising the amino acid sequence set forth in SEQ ID NO: 7. In some aspects, the antibody or antigen-binding portion thereof comprises a VH comprising the amino acid sequence set forth in SEQ ID NO: 97. In some aspects, the antibody or antigen-binding portion thereof comprises a VH comprising the amino acid sequence set forth in SEQ ID NO: 117. In some aspects, the antibody or antigen-binding portion thereof comprises a VH comprising the amino acid sequence set forth in SEQ ID NO: 147. In some aspects, the antibody or antigen-binding portion thereof comprises a VH comprising the amino acid sequence set forth in SEQ ID NO: 217. In some aspects, the antibody or antigen-binding portion thereof comprises a VH comprising the amino acid sequence set forth in SEQ ID NO: 227. In some aspects, the antibody or antigen-binding portion thereof comprises a VH comprising the amino acid sequence set forth in SEQ ID NO: 277. In some aspects, the antibody or antigen-binding portion thereof comprises a VH comprising the amino acid sequence set forth in SEQ ID NO: 287. In some aspects, the antibody or antigen-binding portion thereof comprises a VH comprising the amino acid sequence set forth in SEQ ID NO: 297. In some aspects, the antibody or antigen-binding portion thereof comprises a VH comprising the amino acid sequence set forth in SEQ ID NO: 307.

In some aspects, the antibody or antigen-binding portion thereof comprises a VL comprising an amino acid sequence selected from SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, and 338. In some aspects, the antibody or antigen-binding portion thereof comprises a VL comprising the amino acid sequence set forth in SEQ ID NO: 8. In some aspects, the antibody or antigen-binding portion thereof comprises a VL comprising the amino acid sequence set forth in SEQ ID NO: 98. In some aspects, the antibody or antigen-binding portion thereof comprises a VL comprising the amino acid sequence set forth in SEQ ID NO: 118. In some aspects, the antibody or antigen-binding portion thereof comprises a VL comprising the amino acid sequence set forth in SEQ ID NO: 148. In some aspects, the antibody or antigen-binding portion thereof comprises a VL comprising the amino acid sequence set forth in SEQ ID NO: 218. In some aspects, the antibody or antigen-binding portion thereof comprises a VL comprising the amino acid sequence set forth in SEQ ID NO: 228. In some aspects, the antibody or antigen-binding portion thereof comprises a VL comprising the amino acid sequence set forth in SEQ ID NO: 278. In some aspects, the antibody or antigen-binding portion thereof comprises a VL comprising the amino acid sequence set forth in SEQ ID NO: 288. In some aspects, the antibody or antigen-binding portion thereof comprises a VL comprising the amino acid sequence set forth in SEQ ID NO: 298. In some aspects, the antibody or antigen-binding portion thereof comprises a VL comprising the amino acid sequence set forth in SEQ ID NO: 308.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 7; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 8.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 7; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 8; wherein the antibody or antigen-binding portion thereof binds the same epitope as a reference antibody, wherein the reference antibody comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 7; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 8; wherein the antibody or antigen-binding portion thereof cross competes for binding to human CD161 with a reference antibody, wherein the reference antibody comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 7; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 8; wherein the antibody or antigen-binding portion thereof comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 97; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 98.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 97; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 98; wherein the antibody or antigen-binding portion thereof binds the same epitope as a reference antibody, wherein the reference antibody comprises a VH-CDR1 comprising the amino acid sequence set forth in SEQ ID NO: 91, a VH-CDR2 comprising the amino acid sequence set forth in SEQ ID NO: 92, a VH-CDR3 comprising the amino acid sequence set forth in SEQ ID NO: 93, a VL-CDR1 comprising the amino acid sequence set forth in SEQ ID NO: 94, a VL-CDR2 comprising the amino acid sequence set forth in SEQ ID NO: 95, and a VL-CDR3 comprising the amino acid sequence set forth in SEQ ID NO: 96.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 97; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 98; wherein the antibody or antigen-binding portion thereof cross competes for binding to human comprising the amino acid sequence set forth in SEQ ID NO: 91, a VH-CDR2 comprising the amino acid sequence set forth in SEQ ID NO: 92, a VH-CDR3 comprising the amino acid sequence set forth in SEQ ID NO: 93, a VL-CDR1 comprising the amino acid sequence set forth in SEQ ID NO: 94, a VL-CDR2 comprising the amino acid sequence set forth in SEQ ID NO: 95, and a VL-CDR3 comprising the amino acid sequence set forth in SEQ ID NO: 96.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 97; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 98; wherein the antibody or antigen-binding portion thereof comprises a VH-CDR1 comprising the amino acid sequence set forth in SEQ ID NO: 91, a VH-CDR2 comprising the amino acid sequence set forth in SEQ ID NO: 92, a VH-CDR3 comprising the amino acid sequence set forth in SEQ ID NO: 93, a VL-CDR1 comprising the amino acid sequence set forth in SEQ ID NO: 94, a VL-CDR2 comprising the amino acid sequence set forth in SEQ ID NO: 95, and a VL-CDR3 comprising the amino acid sequence set forth in SEQ ID NO: 96.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 117; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 118.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 117; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 118; wherein the antibody or antigen-binding portion thereof binds the same epitope as a reference antibody, wherein the reference antibody comprises a VH-CDR1 comprising the amino acid sequence set forth in SEQ ID NO: 111, a VH-CDR2 comprising the amino acid sequence set forth in SEQ ID NO: 112, a VH-CDR3 comprising the amino acid sequence set forth in SEQ ID NO: 113, a VL-CDR1 comprising the amino acid sequence set forth in SEQ ID NO: 114, a VL-CDR2 comprising the amino acid sequence set forth in SEQ ID NO: 115, and a VL-CDR3 comprising the amino acid sequence set forth in SEQ ID NO: 116.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 117; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 118; wherein the antibody or antigen-binding portion thereof cross competes for binding to human CD161 with a reference antibody, wherein the reference antibody comprises a VH-CDR1 comprising the amino acid sequence set forth in SEQ ID NO: 111, a VH-CDR2 comprising the amino acid sequence set forth in SEQ ID NO: 112, a VH-CDR3 comprising the amino acid sequence set forth in SEQ ID NO: 113, a VL-CDR1 comprising the amino acid sequence set forth in SEQ ID NO: 114, a VL-CDR2 comprising the amino acid sequence set forth in SEQ ID NO: 115, and a VL-CDR3 comprising the amino acid sequence set forth in SEQ ID NO: 116.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 117; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 118; wherein the antibody or antigen-binding portion thereof comprises a VH-CDR1 comprising the amino acid sequence set forth in SEQ ID NO: 111, a VH-CDR2 comprising the amino acid sequence set forth in SEQ ID NO: 112, a VH-CDR3 comprising the amino acid sequence set forth in SEQ ID NO: 113, a VL-CDR1 comprising the amino acid sequence set forth in SEQ ID NO: 114, a VL-CDR2 comprising the amino acid sequence set forth in SEQ ID NO: 115, and a VL-CDR3 comprising the amino acid sequence set forth in SEQ ID NO: 116.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 147; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 148.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 147; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 148; wherein the antibody or antigen-binding portion thereof binds the same epitope as a reference antibody, wherein the reference antibody comprises a VH-CDR1 comprising the amino acid sequence set forth in SEQ ID NO: 141, a VH-CDR2 comprising the amino acid sequence set forth in SEQ ID NO: 142, a VH-CDR3 comprising the amino acid sequence set forth in SEQ ID NO: 143, a VL-CDR1 comprising the amino acid sequence set forth in SEQ ID NO: 144, a VL-CDR2 comprising the amino acid sequence set forth in SEQ ID NO: 145, and a VL-CDR3 comprising the amino acid sequence set forth in SEQ ID NO: 146.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 147; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 148; wherein the antibody or antigen-binding portion thereof cross competes for binding to human comprising the amino acid sequence set forth in SEQ ID NO: 141, a VH-CDR2 comprising the amino acid sequence set forth in SEQ ID NO: 142, a VH-CDR3 comprising the amino acid sequence set forth in SEQ ID NO: 143, a VL-CDR1 comprising the amino acid sequence set forth in SEQ ID NO: 144, a VL-CDR2 comprising the amino acid sequence set forth in SEQ ID NO: 145, and a VL-CDR3 comprising the amino acid sequence set forth in SEQ ID NO: 146.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 147; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 148; wherein the antibody or antigen-binding portion thereof comprises a VH-CDR1 comprising the amino acid sequence set forth in SEQ ID NO: 141, a VH-CDR2 comprising the amino acid sequence set forth in SEQ ID NO: 142, a VH-CDR3 comprising the amino acid sequence set forth in SEQ ID NO: 143, a VL-CDR1 comprising the amino acid sequence set forth in SEQ ID NO: 144, a VL-CDR2 comprising the amino acid sequence set forth in SEQ ID NO: 145, and a VL-CDR3 comprising the amino acid sequence set forth in SEQ ID NO: 146.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 217; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 218.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 217; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 218; wherein the antibody or antigen-binding portion thereof binds the same epitope as a reference antibody, wherein the reference antibody comprises a VH-CDR1 comprising the amino acid sequence set forth in SEQ ID NO: 211, a VH-CDR2 comprising the amino acid sequence set forth in SEQ ID NO: 212, a VH-CDR3 comprising the amino acid sequence set forth in SEQ ID NO: 213, a VL-CDR1 comprising the amino acid sequence set forth in SEQ ID NO: 214, a VL-CDR2 comprising the amino acid sequence set forth in SEQ ID NO: 215, and a VL-CDR3 comprising the amino acid sequence set forth in SEQ ID NO: 216.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 217; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 218; wherein the antibody or antigen-binding portion thereof cross competes for binding to human CD161 with a reference antibody, wherein the reference antibody comprises a VH-CDR1 comprising the amino acid sequence set forth in SEQ ID NO: 211, a VH-CDR2 comprising the amino acid sequence set forth in SEQ ID NO: 212, a VH-CDR3 comprising the amino acid sequence set forth in SEQ ID NO: 213, a VL-CDR1 comprising the amino acid sequence set forth in SEQ ID NO: 214, a VL-CDR2 comprising the amino acid sequence set forth in SEQ ID NO: 215, and a VL-CDR3 comprising the amino acid sequence set forth in SEQ ID NO: 216.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 217; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 218; wherein the antibody or antigen-binding portion thereof comprises a VH-CDR1 comprising the amino acid sequence set forth in SEQ ID NO: 211, a VH-CDR2 comprising the amino acid sequence set forth in SEQ ID NO: 212, a VH-CDR3 comprising the amino acid sequence set forth in SEQ ID NO: 213, a VL-CDR1 comprising the amino acid sequence set forth in SEQ ID NO: 214, a VL-CDR2 comprising the amino acid sequence set forth in SEQ ID NO: 215, and a VL-CDR3 comprising the amino acid sequence set forth in SEQ ID NO: 216.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 227; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 228.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 227; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 228; wherein the antibody or antigen-binding portion thereof binds the same epitope as a reference antibody, wherein the reference antibody comprises a VH-CDR1 comprising the amino acid sequence set forth in SEQ ID NO: 221, a VH-CDR2 comprising the amino acid sequence set forth in SEQ ID NO: 222, a VH-CDR3 comprising the amino acid sequence set forth in SEQ ID NO: 223, a VL-CDR1 comprising the amino acid sequence set forth in SEQ ID NO: 224, a VL-CDR2 comprising the amino acid sequence set forth in SEQ ID NO: 225, and a VL-CDR3 comprising the amino acid sequence set forth in SEQ ID NO: 226.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 227; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 228; wherein the antibody or antigen-binding portion thereof cross competes for binding to human comprising the amino acid sequence set forth in SEQ ID NO: 221, a VH-CDR2 comprising the amino acid sequence set forth in SEQ ID NO: 222, a VH-CDR3 comprising the amino acid sequence set forth in SEQ ID NO: 223, a VL-CDR1 comprising the amino acid sequence set forth in SEQ ID NO: 224, a VL-CDR2 comprising the amino acid sequence set forth in SEQ ID NO: 225, and a VL-CDR3 comprising the amino acid sequence set forth in SEQ ID NO: 226.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 227; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 228; wherein the antibody or antigen-binding portion thereof comprises a VH-CDR1 comprising the amino acid sequence set forth in SEQ ID NO: 221, a VH-CDR2 comprising the amino acid sequence set forth in SEQ ID NO: 222, a VH-CDR3 comprising the amino acid sequence set forth in SEQ ID NO: 223, a VL-CDR1 comprising the amino acid sequence set forth in SEQ ID NO: 224, a VL-CDR2 comprising the amino acid sequence set forth in SEQ ID NO: 225, and a VL-CDR3 comprising the amino acid sequence set forth in SEQ ID NO: 226.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 277; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 278.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 277; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 278; wherein the antibody or antigen-binding portion thereof binds the same epitope as a reference antibody, wherein the reference antibody comprises a VH-CDR1 comprising the amino acid sequence set forth in SEQ ID NO: 271, a VH-CDR2 comprising the amino acid sequence set forth in SEQ ID NO: 272, a VH-CDR3 comprising the amino acid sequence set forth in SEQ ID NO: 273, a VL-CDR1 comprising the amino acid sequence set forth in SEQ ID NO: 274, a VL-CDR2 comprising the amino acid sequence set forth in SEQ ID NO: 275, and a VL-CDR3 comprising the amino acid sequence set forth in SEQ ID NO: 276.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 277; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 278; wherein the antibody or antigen-binding portion thereof cross competes for binding to human CD161 with a reference antibody, wherein the reference antibody comprises a VH-CDR1 comprising the amino acid sequence set forth in SEQ ID NO: 271, a VH-CDR2 comprising the amino acid sequence set forth in SEQ ID NO: 272, a VH-CDR3 comprising the amino acid sequence set forth in SEQ ID NO: 273, a VL-CDR1 comprising the amino acid sequence set forth in SEQ ID NO: 274, a VL-CDR2 comprising the amino acid sequence set forth in SEQ ID NO: 275, and a VL-CDR3 comprising the amino acid sequence set forth in SEQ ID NO: 276.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 277; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 278; wherein the antibody or antigen-binding portion thereof comprises a VH-CDR1 comprising the amino acid sequence set forth in SEQ ID NO: 271, a VH-CDR2 comprising the amino acid sequence set forth in SEQ ID NO: 272, a VH-CDR3 comprising the amino acid sequence set forth in SEQ ID NO: 273, a VL-CDR1 comprising the amino acid sequence set forth in SEQ ID NO: 274, a VL-CDR2 comprising the amino acid sequence set forth in SEQ ID NO: 275, and a VL-CDR3 comprising the amino acid sequence set forth in SEQ ID NO: 276.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 287; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 288.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 287; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 288; wherein the antibody or antigen-binding portion thereof binds the same epitope as a reference antibody, wherein the reference antibody comprises a VH-CDR1 comprising the amino acid sequence set forth in SEQ ID NO: 281, a VH-CDR2 comprising the amino acid sequence set forth in SEQ ID NO: 282, a VH-CDR3 comprising the amino acid sequence set forth in SEQ ID NO: 283, a VL-CDR1 comprising the amino acid sequence set forth in SEQ ID NO: 284, a VL-CDR2 comprising the amino acid sequence set forth in SEQ ID NO: 285, and a VL-CDR3 comprising the amino acid sequence set forth in SEQ ID NO: 286.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 287; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 288; wherein the antibody or antigen-binding portion thereof cross competes for binding to human comprising the amino acid sequence set forth in SEQ ID NO: 281, a VH-CDR2 comprising the amino acid sequence set forth in SEQ ID NO: 282, a VH-CDR3 comprising the amino acid sequence set forth in SEQ ID NO: 283, a VL-CDR1 comprising the amino acid sequence set forth in SEQ ID NO: 284, a VL-CDR2 comprising the amino acid sequence set forth in SEQ ID NO: 285, and a VL-CDR3 comprising the amino acid sequence set forth in SEQ ID NO: 286.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 287; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 288; wherein the antibody or antigen-binding portion thereof comprises a VH-CDR1 comprising the amino acid sequence set forth in SEQ ID NO: 281, a VH-CDR2 comprising the amino acid sequence set forth in SEQ ID NO: 282, a VH-CDR3 comprising the amino acid sequence set forth in SEQ ID NO: 283, a VL-CDR1 comprising the amino acid sequence set forth in SEQ ID NO: 284, a VL-CDR2 comprising the amino acid sequence set forth in SEQ ID NO: 285, and a VL-CDR3 comprising the amino acid sequence set forth in SEQ ID NO: 286.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 297; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 298.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 297; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 298; wherein the antibody or antigen-binding portion thereof binds the same epitope as a reference antibody, wherein the reference antibody comprises a VH-CDR1 comprising the amino acid sequence set forth in SEQ ID NO: 291, a VH-CDR2 comprising the amino acid sequence set forth in SEQ ID NO: 292, a VH-CDR3 comprising the amino acid sequence set forth in SEQ ID NO: 293, a VL-CDR1 comprising the amino acid sequence set forth in SEQ ID NO: 294, a VL-CDR2 comprising the amino acid sequence set forth in SEQ ID NO: 295, and a VL-CDR3 comprising the amino acid sequence set forth in SEQ ID NO: 296.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 297; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 298; wherein the antibody or antigen-binding portion thereof cross competes for binding to human CD161 with a reference antibody, wherein the reference antibody comprises a VH-CDR1 comprising the amino acid sequence set forth in SEQ ID NO: 291, a VH-CDR2 comprising the amino acid sequence set forth in SEQ ID NO: 292, a VH-CDR3 comprising the amino acid sequence set forth in SEQ ID NO: 293, a VL-CDR1 comprising the amino acid sequence set forth in SEQ ID NO: 294, a VL-CDR2 comprising the amino acid sequence set forth in SEQ ID NO: 295, and a VL-CDR3 comprising the amino acid sequence set forth in SEQ ID NO: 296.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 297; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 298; wherein the antibody or antigen-binding portion thereof comprises a VH-CDR1 comprising the amino acid sequence set forth in SEQ ID NO: 291, a VH-CDR2 comprising the amino acid sequence set forth in SEQ ID NO: 292, a VH-CDR3 comprising the amino acid sequence set forth in SEQ ID NO: 293, a VL-CDR1 comprising the amino acid sequence set forth in SEQ ID NO: 294, a VL-CDR2 comprising the amino acid sequence set forth in SEQ ID NO: 295, and a VL-CDR3 comprising the amino acid sequence set forth in SEQ ID NO: 296.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 307; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 308.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 307; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 308; wherein the antibody or antigen-binding portion thereof binds the same epitope as a reference antibody, wherein the reference antibody comprises a VH-CDR1 comprising the amino acid sequence set forth in SEQ ID NO: 301, a VH-CDR2 comprising the amino acid sequence set forth in SEQ ID NO: 302, a VH-CDR3 comprising the amino acid sequence set forth in SEQ ID NO: 303, a VL-CDR1 comprising the amino acid sequence set forth in SEQ ID NO: 304, a VL-CDR2 comprising the amino acid sequence set forth in SEQ ID NO: 305, and a VL-CDR3 comprising the amino acid sequence set forth in SEQ ID NO: 306.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 307; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 308; wherein the antibody or antigen-binding portion thereof cross competes for binding to human comprising the amino acid sequence set forth in SEQ ID NO: 301, a VH-CDR2 comprising the amino acid sequence set forth in SEQ ID NO: 302, a VH-CDR3 comprising the amino acid sequence set forth in SEQ ID NO: 303, a VL-CDR1 comprising the amino acid sequence set forth in SEQ ID NO: 304, a VL-CDR2 comprising the amino acid sequence set forth in SEQ ID NO: 305, and a VL-CDR3 comprising the amino acid sequence set forth in SEQ ID NO: 306.

In some aspects, the antibody or antigen-binding portion thereof comprises (i) a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 307; and (ii) a VL comprising an amino acid sequence having at least about 80%, at least about 85%, at least about 90%, at least about 95%, at least about 96%, at least about 97%, at least about 98%, or at least about 99% sequence identity to the amino acid sequence selected set forth in SEQ ID NO: 308; wherein the antibody or antigen-binding portion thereof comprises a VH-CDR1 comprising the amino acid sequence set forth in SEQ ID NO: 301, a VH-CDR2 comprising the amino acid sequence set forth in SEQ ID NO: 302, a VH-CDR3 comprising the amino acid sequence set forth in SEQ ID NO: 303, a VL-CDR1 comprising the amino acid sequence set forth in SEQ ID NO: 304, a VL-CDR2 comprising the amino acid sequence set forth in SEQ ID NO: 305, and a VL-CDR3 comprising the amino acid sequence set forth in SEQ ID NO: 306.

In some aspects, the antibody or antigen-binding portion thereof comprises a VH comprising the amino acid sequence set forth in SEQ ID NO: 7, and a VL comprising the amino acid sequence set forth in SEQ ID NO: 8. In some aspects, the antibody or antigen-binding portion thereof binds the same epitope as a reference antibody, wherein the reference antibody comprises a VH comprising the amino acid sequence set forth in SEQ ID NO: 7, and a VL comprising the amino acid sequence set forth in SEQ ID NO: 8. In some aspects, the antibody or antigen-binding portion thereof cross competes for binding to human CD161 with a reference antibody, wherein the reference antibody comprises a VH comprising the amino acid sequence set forth in SEQ ID NO: 7, and a VL comprising the amino acid sequence set forth in SEQ ID NO: 8.

In some aspects, the antibody or antigen-binding portion thereof comprises a VH comprising the amino acid sequence set forth in SEQ ID NO: 97, and a VL comprising the amino acid sequence set forth in SEQ ID NO: 98. In some aspects, the antibody or antigen-binding portion thereof binds the same epitope as a reference antibody, wherein the reference antibody comprises a VH comprising the amino acid sequence set forth in SEQ ID NO: 97, and a VL comprising the amino acid sequence set forth in SEQ ID NO: 98. In some aspects, the antibody or antigen-binding portion thereof cross competes for binding to human CD161 with a reference antibody, wherein the reference antibody comprises a VH comprising the amino acid sequence set forth in SEQ ID NO: 97, and a VL comprising the amino acid sequence set forth in SEQ ID NO: 98.

In some aspects, the antibody or antigen-binding portion thereof comprises a VH comprising the amino acid sequence set forth in SEQ ID NO: 117, and a VL comprising the amino acid sequence set forth in SEQ ID NO: 118. In some aspects, the antibody or antigen-binding portion thereof binds the same epitope as a reference antibody, wherein the reference antibody comprises a VH comprising the amino acid sequence set forth in SEQ ID NO: 117, and a VL comprising the amino acid sequence set forth in SEQ ID NO: 118. In some aspects, the antibody or antigen-binding portion thereof cross competes for binding to human CD161 with a reference antibody, wherein the reference antibody comprises a VH comprising the amino acid sequence set forth in SEQ ID NO: 117, and a VL comprising the amino acid sequence set forth in SEQ ID NO: 118.

In some aspects, the antibody or antigen-binding portion thereof comprises a VH comprising the amino acid sequence set forth in SEQ ID NO: 147, and a VL comprising the amino acid sequence set forth in SEQ ID NO: 148. In some aspects, the antibody or antigen-binding portion thereof binds the same epitope as a reference antibody, wherein the reference antibody comprises a VH comprising the amino acid sequence set forth in SEQ ID NO: 147, and a VL comprising the amino acid sequence set forth in SEQ ID NO: 148. In some aspects, the antibody or antigen-binding portion thereof cross competes for binding to human CD161 with a reference antibody, wherein the reference antibody comprises a VH comprising the amino acid sequence set forth in SEQ ID NO: 147, and a VL comprising the amino acid sequence set forth in SEQ ID NO: 148.

In some aspects, the antibody or antigen-binding portion thereof comprises a VH comprising the amino acid sequence set forth in SEQ ID NO: 217, and a VL comprising the amino acid sequence set forth in SEQ ID NO: 218. In some aspects, the antibody or antigen-binding portion thereof binds the same epitope as a reference antibody, wherein the reference antibody comprises a VH comprising the amino acid sequence set forth in SEQ ID NO: 217, and a VL comprising the amino acid sequence set forth in SEQ ID NO: 218. In some aspects, the antibody or antigen-binding portion thereof cross competes for binding to human CD161 with a reference antibody, wherein the reference antibody comprises a VH comprising the amino acid sequence set forth in SEQ ID NO: 217, and a VL comprising the amino acid sequence set forth in SEQ ID NO: 218.

In some aspects, the antibody or antigen-binding portion thereof comprises a VH comprising the amino acid sequence set forth in SEQ ID NO: 227, and a VL comprising the amino acid sequence set forth in SEQ ID NO: 228. In some aspects, the antibody or antigen-binding portion thereof binds the same epitope as a reference antibody, wherein the reference antibody comprises a VH comprising the amino acid sequence set forth in SEQ ID NO: 227, and a VL comprising the amino acid sequence set forth in SEQ ID NO: 228. In some aspects, the antibody or antigen-binding portion thereof cross competes for binding to human CD161 with a reference antibody, wherein the reference antibody comprises a VH comprising the amino acid sequence set forth in SEQ ID NO: 227, and a VL comprising the amino acid sequence set forth in SEQ ID NO: 228.

In some aspects, the antibody or antigen-binding portion thereof comprises a VH comprising the amino acid sequence set forth in SEQ ID NO: 277, and a VL comprising the amino acid sequence set forth in SEQ ID NO: 278. In some aspects, the antibody or antigen-binding portion thereof binds the same epitope as a reference antibody, wherein the reference antibody comprises a VH comprising the amino acid sequence set forth in SEQ ID NO: 277, and a VL comprising the amino acid sequence set forth in SEQ ID NO: 278. In some aspects, the antibody or antigen-binding portion thereof cross competes for binding to human CD161 with a reference antibody, wherein the reference antibody comprises a VH comprising the amino acid sequence set forth in SEQ ID NO: 277, and a VL comprising the amino acid sequence set forth in SEQ ID NO: 278.

In some aspects, the antibody or antigen-binding portion thereof comprises a VH comprising the amino acid sequence set forth in SEQ ID NO: 287, and a VL comprising the amino acid sequence set forth in SEQ ID NO: 288. In some aspects, the antibody or antigen-binding portion thereof binds the same epitope as a reference antibody, wherein the reference antibody comprises a VH comprising the amino acid sequence set forth in SEQ ID NO: 287, and a VL comprising the amino acid sequence set forth in SEQ ID NO: 288. In some aspects, the antibody or antigen-binding portion thereof cross competes for binding to human CD161 with a reference antibody, wherein the reference antibody comprises a VH comprising the amino acid sequence set forth in SEQ ID NO: 287, and a VL comprising the amino acid sequence set forth in SEQ ID NO: 288.

In some aspects, the antibody or antigen-binding portion thereof comprises a VH comprising the amino acid sequence set forth in SEQ ID NO: 297, and a VL comprising the amino acid sequence set forth in SEQ ID NO: 298. In some aspects, the antibody or antigen-binding portion thereof binds the same epitope as a reference antibody, wherein the reference antibody comprises a VH comprising the amino acid sequence set forth in SEQ ID NO: 297, and a VL comprising the amino acid sequence set forth in SEQ ID NO: 298. In some aspects, the antibody or antigen-binding portion thereof cross competes for binding to human CD161 with a reference antibody, wherein the reference antibody comprises a VH comprising the amino acid sequence set forth in SEQ ID NO: 297, and a VL comprising the amino acid sequence set forth in SEQ ID NO: 298.

In some aspects, the antibody or antigen-binding portion thereof comprises a VH comprising the amino acid sequence set forth in SEQ ID NO: 307, and a VL comprising the amino acid sequence set forth in SEQ ID NO: 308. In some aspects, the antibody or antigen-binding portion thereof binds the same epitope as a reference antibody, wherein the reference antibody comprises a VH comprising the amino acid sequence set forth in SEQ ID NO: 307, and a VL comprising the amino acid sequence set forth in SEQ ID NO: 308. In some aspects, the antibody or antigen-binding portion thereof cross competes for binding to human CD161 with a reference antibody, wherein the reference antibody comprises a VH comprising the amino acid sequence set forth in SEQ ID NO: 307, and a VL comprising the amino acid sequence set forth in SEQ ID NO: 308.

In some aspects, the antibody or antigen-binding portion thereof comprises one or more post-translation modification. In some aspects, the antibody or antigen-binding portion thereof comprises one or more post-translation modification that increases the in vivo half-life of the antibody or antigen-binding portion thereof. In some aspects, the antibody or antigen-binding portion thereof is pegylated.

In some aspects, the antigen-binding portion of the antibody comprises a VHH, a vNAR, a microbody, a nanobody, an scFv, or any combination thereof.

### II.B. Bispecific and Multispecific Antibodies

Anti-CD161 antibodies and antigen-binding portions thereof described herein can be used for forming bispecific and multispecific molecules. An anti-CD161 antibody, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. For example, an anti-CD161 antibody can be linked to an antibody or scFv that binds specifically to a tumor antigen. The antibody described herein can in fact be derived or linked to more than one other functional molecule to generate multispecific molecules that bind to more than two different binding sites and/or target molecules. To create a bispecific molecule described herein, an antibody described herein can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, provided herein are bispecific molecules comprising at least one first binding specificity for CD161 and a second binding specificity for a second target epitope. Further provided herein are multispecific molecules comprising at least one first binding specificity for CD161, a second binding specificity for a second target epitope, and a third binding specificity for a third target epitope.

In some aspects, the bispecific and multispecific molecules described herein comprise as a binding specificity at least one antibody, or an antibody fragment thereof, including, e.g., an Fab, Fab′, F(ab′) 2, Fv, or a single chain Fv (scFv). The antibody can also be a light chain or heavy chain dimer, or any minimal fragment thereof such as a Fv or a single chain construct as described in Ladner et al. U.S. Pat. No. 4,946,778.

While human monoclonal antibodies are preferred, other antibodies which can be employed in the bispecific and multispecific molecules described herein are murine, chimeric and humanized monoclonal antibodies.

The bispecific and multispecific molecules described herein can be prepared by conjugating the constituent binding specificities using methods known in the art. For example, each binding specificity of the bispecific or multispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio) propionate (SPDP), and sulfosuccinimidyl 4-(N-maleimidomethyl) cyclohaxane-1-carboxylate (sulfo-SMCC) (see, e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139:2367-2375). Some conjugating agents are SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In some aspects, the hinge region is modified to contain an odd number of sulfhydryl residues, preferably one, prior to conjugation.

Alternatively, the multiple binding specificities can be encoded in the same vector and expressed and assembled in the same host cell. This method is particularly useful where the bispecific molecule is a mAb×mAb, mAb×Fab, mAb×(scFv)₂, Fab×F(ab′)₂ or ligand x Fab fusion protein. A bispecific antibody can comprise an antibody comprising an scFv at the C-terminus of each heavy chain. A bispecific molecule described herein can be a single chain molecule comprising one single chain antibody and a binding determinant, or a single chain bispecific molecule comprising two binding determinants. Bispecific molecules can comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific and multispecific molecules to their specific targets can be confirmed using art-recognized methods, such as enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

In some aspects, the bispecific antibody comprises (i) an anti-CD161 antibody or antigen-binding portion thereof disclosed herein and (ii) an antibody or an antigen-binding portion thereof that specifically binds a tumor antigen. In some aspects, the tumor antigen is selected from CD19, TRAC, TCRβ, BCMA, CLL-1, CS1, CD38, CD19, TSHR, CD123, CD22, CD30, CD70, CD171, CD33, EGFRVIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-1a, MAGE-Al, legumain, HPV E6,E7, MAGE AI, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin BI, MYCN, RhoC, TRP-2, CYP1B1, BORIS, SART3, PAX5, OY-TESI, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD38, CD4, CD5, CD7, the extracellular portion of the APRIL protein, or any combinations thereof. In some aspects, the TCR targets AFP, CD19, TRAC, TCRβ, BCMA, CLL-1, CS1, CD38, CD19, TSHR, CD123, CD22, CD30, CD171, CD33, EGFRVIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR, ADRB3, PANX3, GPR20, LY6K, OR51E2, TARP, WTI, NY-ESO-1, LAGE-1a, MAGE-Al, legumain, HPV E6,E7, MAGE AI, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin BI, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD38, CD4, CD5, CD7, the extracellular portion of the APRIL protein, and any combinations thereof. In some aspects, the bispecific antibody comprises (i) an anti-CD161 antibody or antigen-binding portion thereof disclosed herein and (ii) an antibody or an antigen-binding portion thereof that specifically binds a tumor antigen selected from CD19, TRAC, TCRβ, BCMA, CLL-1, CS1, CD38, CD19, TSHR, CD123, CD22, CD30, CD70, CD171, CD33, EGFRVIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 IRa, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR, ADRB3, PANX3, GPR20, LY6K, OR51E2, TARP, WTI, NY-ESO-1, LAGE-1a, MAGE-Al, legumain, HPV E6,E7, MAGE AI, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin BI, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD38, CD4, CD5, CD7, the extracellular portion of the APRIL protein, or any combinations thereof. In some aspects, the TCR targets AFP, CD19, TRAC, TCRβ, BCMA, CLL-1, CS1, CD38, CD19, TSHR, CD123, CD22, CD30, CD171, CD33, EGFRVIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-1a, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin BI, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD38, CD4, CD5, CD7, the extracellular portion of the APRIL protein, and any combinations thereof.

### II.C. Nucleic Acid Molecules

Another aspect described herein pertains to nucleic acid molecules that encode the anti-CD161 antibodies described herein. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In some aspects, the nucleic acid is a cDNA molecule.

Nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Some nucleic acids molecules described herein are those encoding the VH and VL sequences of the anti-CD161 antibodies disclosed herein.

The nucleic acid molecules of the present disclosure can be modified to delete specific sequences, e.g., restriction enzyme recognition sequences, or to optimize codons.

A method for making the anti-CD161 antibodies disclosed herein can comprise expressing the heavy chain and the light chains in a cell line comprising the nucleotide sequences encoding the heavy and light chains with a signal peptide. Host cells comprising these nucleotide sequences are encompassed herein.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2, and/or CH3). The sequences of human heavy chain constant region genes are known in the art (see, e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, for example, an IgG1 region. For a Fab fragment heavy chain gene, the VH-encoding DNA can be operatively linked to another DNA molecule encoding only the heavy chain CHI constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see, e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker.

Some aspects of the present disclosure are directed to a vector or a set of vectors comprising a nucleic acid molecule disclosed herein. In some aspects, the vector is a viral vector. In some aspects, the vector is a viral particle or a virus. In some aspects, the vector is a mammalian vector. In some aspects, the vector is a bacterial vector.

In certain aspects, the vector is a retroviral vector. In some aspects, the vector is selected from an adenoviral vector, a lentivirus, a Sendai virus, a baculoviral vector, an Epstein Barr viral vector, a papovaviral vector, a vaccinia viral vector, a herpes simplex viral vector, and an adeno associated virus (AAV) vector. In particular aspects, the vector is an AAV vector. In some aspects, the vector is a lentivirus. In particular aspects, the vector is an AAV vector. In some aspects, the vector is a Sendai virus. In some aspects, the vector is a hybrid vector. Examples of hybrid vectors that can be used in the present disclosure can be found in Huang and Kamihira, Biotechnol. Adv. 31 (2): 208-23 (2103), which is incorporated by reference herein in its entirety.

Some aspects of the present disclosure are directed to a host cell comprising a nucleic acid molecule, a set of nucleic acid molecules, a vector, or a set of vectors disclosed herein. In some aspects, the host cells is a mammalian cell. In some aspects, the host cell is an in vitro cell.

### II.D. Pharmaceutical Compositions

Further provided are compositions, e.g., a pharmaceutical compositions, comprising an anti-CD161 antibody, a nucleic acid molecule, a vector, or a host cell disclosed herein and one or more pharmaceutically acceptable carriers.

In some aspects, the composition further comprises a bulking agent. A bulking agent can be selected from the group consisting of NaCl, mannitol, glycine, alanine, and any combination thereof. In other aspects, the composition comprises a stabilizing agent. The stabilizing agent can be selected from the group consisting of sucrose, trehalose, raffinose, arginine; or any combination thereof. In other aspects, the composition comprises a surfactant. In some aspects, the surfactant is selected from polysorbate 80 (PS80), polysorbate 20 (PS20), and any combination thereof. In certain aspects, the composition further comprises a chelating agent. In some aspects, the chelating agent is selected from diethylenetriaminepentaacetic acid (DTPA), ethylenediaminetetraacetic acid, nitrilotriacetic acid, and any combination thereof. In some aspects, the composition further comprises NaCl, mannitol, pentetic acid (DTPA), sucrose, PS80, or any combination thereof.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition described herein can also include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that can be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition can comprise a preservative or can be devoid of a preservative. Supplementary active compounds can be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

A composition described herein can be administered via one or more routes of administration using one or more of a variety of methods known in the art. As will be appreciated by the skilled artisan, the route and/or mode of administration will vary depending upon the desired results. Routes of administration for the anti-CD161 antibodies described herein can include intravenous, intramuscular, subcutaneous, intraperitoneal, spinal or other parenteral routes of administration, for example by injection or infusion.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

### III. Methods of the Disclosure

### III.A. Methods of Treatment

Some aspects of the present disclosure are directed to method of treating a disease or disorder in a subject, comprising administering to the subject an anti-CD161 antibody disclosed herein, a polynucleotide encoding the anti-CD161 antibody, a vector comprising the polynucleotide, a host cell comprising the polynucleotide, or any combination thereof. In some aspects, the disease or disorder comprises a cancer.

Some aspects of the present disclosure are directed to a method of treating a cancer in a subject in need thereof, comprising administering to the subject an effective dose of a composition disclosed herein (e.g., an antibody, polynucleotide, vector, host cell, or pharmaceutical composition). In other aspects, the present disclosure is directed to a method of killing a tumor cell in a subject in need thereof, comprising administering to the subject an effective dose of a composition disclosed herein. In other aspects, the present disclosure is directed to a method of reducing the size of a tumor in a subject in need thereof, comprising administering to the subject an effective dose of a composition disclosed herein. In other aspects, the present disclosure is directed to inhibiting metastasis of a tumor in a subject in need thereof, comprising administering to the subject an effective dose of a composition disclosed herein. In some aspects, the subject is a human.

Some aspects of the present disclosure are directed to methods of inducing an immune response in a subject in need thereof, comprising administering to the subject an anti-CD161 antibody disclosed herein, a polynucleotide encoding the anti-CD161 antibody, a vector comprising the polynucleotide, a host cell comprising the polynucleotide, or any combination thereof. In some aspects, the subject is afflicted with a cancer.

Some aspects of the present disclosure are directed to a method of activating an immune cell, comprising contacting the immune cell with an anti-CD161 antibody disclosed herein, a polynucleotide encoding the anti-CD161 antibody, a vector comprising the polynucleotide, a host cell comprising the polynucleotide, or any combination thereof. In some aspects, the immune cell is contacted in vitro. In some aspects, the immune cell is contacted ex vivo. In some aspects, the immune cell is present in a human subject, i.e., the immune cell is contacted in vivo. In some aspects, the subject is afflicted with a cancer.

In some aspects, the cancer comprises a solid tumor. In some aspects, the cancer comprises a hematological malignancy. In some aspects, the cancer is locally advanced. In some aspects, the cancer is metastasized. In some aspects, the tumor is recurrent. In some aspects, the tumor is refractory. In some aspects, the tumor is recurrent and/or refractory following one or more prior therapy to treat the tumor. In some aspects, the one or more prior therapy comprises a standard of care therapy. In some aspects, the one or more prior therapy comprises a chemotherapy. In some aspects, the one or more prior therapy comprises an immunotherapy. In some aspects, the one or more prior therapy comprises a surgery. In some aspects, the one or more prior therapy comprises a radiotherapy.

In some aspects, the cancer is selected from acoustic neuroma, acute lymphocytic leukemia, acute myelocytic leukemia, adenocarcinoma, and cancer of the urinary system, and carcinomas, angiosarcoma, astrocytoma, basal cell carcinoma, bile duct carcinoma, biliary tract cancer, bladder cancer, bone cancer, brain cancer, brain stem glioma, breast cancer, bronchogenic carcinoma, Burkitt's lymphoma and marginal zone B cell lymphoma, cancer of the adrenal gland, cancer of the anal region, cancer of the digestive system, cancer of the endocrine system, cancer of the esophagus, cancer of the parathyroid gland, cancer of the penis, cancer of the respiratory system, cancer of the small intestine, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, central nervous system (CNS) cancer, cervical cancer, chondrosarcoma, chordoma, choriocarcinoma, chronic leukemia, chronic lymphocytic leukemia, chronic myelocytic (granulocytic) leukemia, colon carcinoma, colon sarcoma, colorectal cancer, connective tissue cancer, craniopharyngioma, cystadenocarcinoma, embryonal carcinoma, endometrial cancer, endotheliosarcoma, environmentally-induced cancers including those induced by asbestos, ependymoma, epidermoid cancer, epithelial carcinoma, esophageal cancer, esophageal carcinoma, Ewing's tumor, eye cancer, fibrosarcoma, gastric cancer, gastrointestinal cancer, germ cell tumor, glioblastoma (e.g. glioblastoma multiforme), glioma, head and neck cancer, heavy chain disease, hemangioblastoma, hepatoma, Hodgkin's disease, intraepithelial neoplasm, Kaposi's sarcoma, kidney cancer (e.g. renal cell carcinoma (RCC)), larynx cancer, leiomyosarcoma, leukemia, liposarcoma, liver cancer, lung cancer (small cell, large cell), lung carcinoma, lymphangioendotheliosarcoma, lymphangiosarcoma, mantle cell lymphoma, medullary carcinoma, medulloblastoma, melanoma, menangioma, mesothelioma, multiple myeloma, myeloblasts promyelocyte myelomonocytic monocytic erythroleukemia, myxosarcoma, nasopharyngeal carcinoma, neoplasm of the central nervous system (CNS), neuroblastoma, non-Hodgkin's disease, non-small cell lung cancer (NSCLC), non-small cell lung carcinoma, oligodendroglioma, oral cavity cancer (for example lip, tongue, mouth and pharynx), osteogenic sarcoma, osteosarcoma, ovarian cancer, pancreatic cancer, papillary adenocarcinomas, papillary carcinoma, pediatric sarcoma, pinealoma, pituitary adenoma, Polycythemia vera Lymphoma, primary CNS lymphoma, prostate cancer (e.g. hormone refractory prostate adenocarcinoma), rectal cancer, renal cancer (e.g. clear cell carcinoma), retinoblastoma, rhabdomyosarcoma, sarcoma, sarcoma of soft tissue, sebaceous gland carcinoma, seminoma, sinonasal natural killer, skin cancer, small-cell lung cancer (SCLC), solid tumors of childhood, spinal axis tumor, squamous cell cancer, squamous cell carcinoma, stomach cancer, sweat gland carcinoma, synovioma, testicular cancer, thyroid cancer, tumor angiogenesis, uterine cancer, virus-related cancers or cancers of viral origin (e.g. human papilloma virus (HPV-related or- originating tumors)), Waldenstrom's macroglobulinemia, and Wilm's tumor; and any combinations of said cancers.

In some aspects, the subject is afflicted with an infectious disease, i.e., in some aspects the disease or disorder comprises an infectious disease. In some aspects, the infectious disease is selected from a bacterial infection, a fungal infection, a viral infection, a parasitic infection, or any combination thereof. In some aspects, the infectious disease comprises infection by Influenza, Herpes, Giardia, Malaria, Leishmania, or any combination thereof. In some aspects, the infectious diseases comprises infection by human immunodeficiency virus (HIV), Hepatitis virus herpes virus, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus, or arboviral encephalitis virus, or any combination thereof. In some aspects, the infectious disease comprises infection by chlamydia, rickettsial bacteria, mycobacteria, staphylococci, streptococci, pneumonococci, meningococci, conococci, klebsiella, proteus, serratia, pseudomonas, legionella, diphtheria, salmonella, bacilli, cholera, tetanus, botulism, anthrax, plague, leptospirosis, and Lyme's disease bacteria, or any combination thereof. In some aspects, the infectious disease comprises infection by Candida, Cryptococcus neoformans, Aspergillus, genus Mucorales, Sporothrix schenkii, Blastomyces dermatitidis, Paracoccidioides brasiliensis, Coccidioides immitis, or Histoplasma capsulatum, or any combination thereof. In some aspects, the infectious disease comprises infection by Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, or Nippostrongylus brasiliensis, or any combination thereof.

In some aspects, the subject is afflicted with an autoimmune disease.

The compositions of the present disclosure can be administered using any pharmaceutically acceptable route. In some aspects, the composition (e.g., antibody, polynucleotide, vector, host cell, or pharmaceutical composition) is administered intravenously, intraperitoneally, intramuscularly, intraarterially, intrathecally, intralymphaticly, intralesionally, intracapsularly, intraorbitally, intracardiacly, intradermally, transtracheally, subcutaneously, subcuticularly, intraarticularly, subcapsularly, subarachnoidly, intraspinally, epidurally, intrasternally, topically, epidermally, mucosally, or any combination thereof. In some aspects, the composition is administered intravenously. In some aspects, the composition is administered subcutaneously.

In certain aspects, the method reduces the size of a cancer, e.g., the size of a tumor, in the subject. In some aspects, the size of the caner is reduced by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%.

In some aspects, the method increases the over survival of the subject. In some aspects, the overall survival is increased relative to the average overall survival of a subject having the same cancer but treated with a different therapy. In certain aspects, the overall survival is increased by at least about 10%, at least about 20%, at least about 25%, at least about 50%, at least about 2 fold, at least about 3 fold, at least about 5 fold. In some aspects, the overall survival is increased by at least about one month, at least about 2 months, at least about 3 months, at least about 4 months, at least about 5 months, at least about 6 months, at least about 7 months, at least about 8 months, at least about 9 months, at least about 10 months, at least about 1 months, at least about 12 months, at least about 15 months, at least about 18 months, at least about 21 months, at least about 2 years, at least about 3 years, at least about 4 years, at least about 5 years, or at least about 10 years.

In some aspects, the method increases the progression free survival of the subject. In some aspects, the overall survival is increased relative to the average progression free survival of a subject having the same cancer but treated with a different therapy. In certain aspects, the progression free survival is increased by at least about 10%, at least about 20%, at least about 25%, at least about 50%, at least about 2 fold, at least about 3 fold, at least about 5 fold. In some aspects, the overall survival is increased by at least about one month, at least about 2 months, at least about 3 months, at least about 4 months, at least about 5 months, at least about 6 months, at least about 7 months, at least about 8 months, at least about 9 months, at least about 10 months, at least about 1 months, at least about 12 months, at least about 15 months, at least about 18 months, at least about 21 months, at least about 2 years, at least about 3 years, at least about 4 years, at least about 5 years, or at least about 10 years.

In some aspects, the method increases the objective response rate of the subject. In certain aspects, the method induces a complete response in the subject. In some aspects, the method induces a partial response in the subject.

In some aspects, the method comprises administering an anti-CD161 antibody (or a polynucleotide, vector, or host cell) disclosed herein and a second therapy. In some aspects, the second therapy is administered prior to the anti-CD161 antibody. In some aspects, the second therapy is administered after the anti-CD161 antibody. In some aspects, the second therapy is administered concurrently with the anti-CD161 antibody. In certain aspects, the anti-CD161 antibody and the second therapy are administered separately. In other aspects, the anti-CD161 antibody and the second therapy are administered in a single formulation.

The second therapy can be any other therapy. In some aspects, the second therapy comprises an immunotherapy. In some aspects, the second therapy comprises a chemotherapy. In some aspects, the second therapy comprises a radiotherapy. In some aspects, the second therapy comprises a surgery. In some aspects, the second therapy comprises administering a second therapeutic agent.

In some aspects, the one or more additional therapeutic agents is a PD-1 antagonist, a TIM-3 inhibitor, a LAG-3 inhibitor, a TIGIT inhibitor, a CD112R inhibitor, a TAM inhibitor, a STING agonist, a 4-1BB agonist, or a combination thereof. In some aspects, the one or more additional therapeutic agents is a CD39 antagonist, a CD73 antagonist, a CCR8 antagonist, or a combination thereof. In some aspects, the anti-CD73 is any anti-CD73 antibody disclosed in, e.g., U.S. Publication No. 2019/0031766 A1, which is incorporated by reference herein in its entirety. In some aspects, the anti-CD39 is any anti-CD39 antibody disclosed in, e.g., Int'l Publication No. WO 2019/178269 A2, which is incorporated by reference herein in its entirety.

In certain aspects, the second therapeutic agent comprises a second antibody. In some aspects, the second therapeutic agent comprises an effective amount of an antibody that specifically binds a protein selected from Inducible T cell Co-Stimulator (ICOS), CD137 (4-1BB), CD134 (OX40), NKG2A, CD27, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), the receptor for V-domain Ig Suppressor of T cell Activation (VISTA), NKG2a, KIR, TGFβ, IL-10, IL-8, B7-H4, Fas ligand, CXCR4, mesothelin, CEACAM-1, CD96, CD52, HER2, and any combination thereof.

An anti-CD161 antibody or an antigen-binding portion thereof described herein can replace or augment a previously or currently administered therapy. For example, upon treating with an anti-CD161 antibody or antigen-binding portion thereof, administration of the one or more additional therapeutics can cease or diminish, e.g., be administered at lower levels. In some aspects, administration of the previous therapy can be maintained. In some aspects, a previous therapy will be maintained until the level of the anti-CD161 antibody reaches a level sufficient to provide a therapeutic effect.

An anti-CD161 antibody or an antigen-binding portion thereof described herein can be used as a diagnostic. In some aspects, the anti-CD161 antibody is used to identify a subject suitable for an immunotherapy. In some aspects, the anti-CD161 antibody is used to identify a subject having expression of CD161 in NK cells.

In some aspects, the one or more additional therapeutic agents is a PD-1 antagonist. In some aspects, the PD-1 antagonist is selected from the group consisting of: PDR001, nivolumab, pembrolizumab, pidilizumab, MEDI0680, REGN2810, TSR-042, PF-06801591, and AMP-224. In certain aspects, the one or more additional therapeutic agents is a PD-L1 inhibitor. In some aspects, the PD-L1 inhibitor is selected from the group consisting of: FAZ053, Atezolizumab, Avelumab, Durvalumab, and BMS-936559. In some aspects, the disclosure provides a method of enhancing one or more activities of an anti-PD-1 antibody (e.g., enhances PD-1-mediated cytokine secretion; enhances anti-PD-1 mediated TNFa secretion; enhances anti-PD-1 mediated IL-6 secretion from a cell exposed to anti-PD-1 antibodies), the method comprising exposing a cell to an antibody, or antigen binding portion thereof, provided by the disclosure, concurrently with or sequentially to an anti-PD-1 antibody, thereby to enhance one or more activities of the anti-PD1 antibody.

In some aspects, the one or more additional therapeutic agents is Sunitinib (Sutent®), Cabozantinib (CABOMETYX®), Axitinib (INLYTAR), Lenvatinib (LENVIMA®), Everolimus (AFINITOR®), Bevacizumab (AVASTIN®), epacadostat, NKTR-214 (CD-122-biased agonist), tivozanib (FOTIVDA®), abexinostat, Ipilimumab (YERVOY®), tremelimumab, Pazopanib (VOTRIENT®), Sorafenib (NEXAVAR®), Temsirolimus (TORISEL®), Ramucirumab (CYRAMZA®), niraparib, savolitinib, vorolanib (X-82), Regorafenib (STIVARGO®), Donafenib (multikinase inhibitor), Camrelizumab (SHR-1210), pexastimogene devacirepvec (JX-594), Ramucirumab (CYRAMZA®), apatinib (YN968D1), encapsulated doxorubicin (THERMODOX®), Tivantinib (ARQ197), ADI-PEG 20, binimetinib, apatinib mesylate, nintedanib, lirilumab, Nivolumab (OPDIVO®), Pembrolizumab (KEYTRUDA®), Atezolizumab (TECENTRIQ®), Avelumab (BAVENCIO®), Durvalumab (IMFIMZI®), Cemiplimab-rwlc (LIBTAYO®), tislelizumab, and/or spartalizumab.

In some aspects, the one or more additional therapeutic agents is a TIM-3 inhibitor, optionally wherein the TIM-3 inhibitor is MGB453 or TSR-022.

In some aspects, the one or more additional therapeutic agents is a LAG-3 inhibitor, optionally wherein the LAG-3 inhibitor is selected from the group consisting of LAG525, BMS-986016, and TSR-033.

In some aspects, the one or more additional therapeutic agents is a TIGIT inhibitor. In some aspects, the one or more additional therapeutic agents is a CD112R inhibitor. In some aspects, the one or more additional therapeutic agents is a TAM (Axl, Mer, Tyro) inhibitor. In some aspects, the one or more additional therapeutic agents is a STING agonist. In some aspects, the one or more additional therapeutic agents is a 4-1BB agonist.

In some aspects, the one or more additional therapeutic agents is a tyrosine kinase inhibitor, an agent targeting the adenosine axis (for example a CD39 antagonist, a CD73 antagonist or a A2AR, A2BR or dual A2AR/A2BR antagonist), a CCR8 antagonist, a CTLA4 antagonist, a VEG-F inhibitor or a combination thereof.

III.A.1. Combination with Chemotherapeutic Agents

Chemotherapeutic agents suitable for combination and/or co-administration with compositions of the present invention include, for example: taxol, cytochalasin B, gramicidin D, ethidium bromide, emetine, mitomycin, etoposide, tenoposide, vincristine, vinblastine, colchicin, doxorubicin, daunorubicin, dihydroxyanthrancindione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g., mechlorethamine, thioTEPA, chlorambucil, melphalan, carmustine (BSNU), lomustine (CCNU), cyclophosphamide, busulfan, dibromomannitol, streptozotocin, mitomycin C, cis-dichlordiamine platinum (II) (DDP), procarbazine, altretamine, cisplatin, carboplatin, oxaliplatin, nedaplatin, satraplatin, or triplatin tetranitrate), anthracycline (e.g., daunorubicin (formerly daunomycin) and doxorubicin), antibiotics (e.g., dactinomcin (formerly actinomycin), bleomycin, mithramycin, and anthramycin (AMC)), and anti-mitotic agents (e.g., vincristine and vinblastine) and temozolomide.

III.A.2. Combination with PD-1/PD-L1 Antagonists

In some aspects, the anti-CD161 antibodies, or antigen binding portions thereof, provided by the disclosure are combined (e.g., administered in combination) with one or more PD-1 antagonist that specifically binds to human PD-1 or PD-L1 and inhibits PD-1/PD-L1 biological activity and/or downstream pathway(s) and/or cellular processed mediated by human PD-1/PD-L1 signaling or other human PD-1/PD-L1-mediated functions.

Accordingly, provided herein are PD-1 antagonists that directly or allosterically block, antagonize, suppress, inhibit or reduce PD-1/PD-L1 biological activity, including downstream pathways and/or cellular processes mediated by PD-1/PD-L1 signaling, such as receptor binding and/or elicitation of a cellular response to PD-1/PD-L1. Also provided herein are PD-1 antagonists that reduce the quantity or amount of human PD-1 or PD-L1 produced by a cell or subject.

In some aspects, the disclosure provides a PD-1 antagonist that binds human PD-1 and prevents, inhibits or reduces PD-L1 binding to PD-1. In some aspects, the PD-1 antagonist binds to the mRNA encoding PD-1 or PD-L1 and prevents translation. In some aspects, the PD-1 antagonist binds to the mRNA encoding PD-1 or PD-L1 and causes degradation and/or turnover.

In some aspects, the PD-1 antagonist inhibits PD-1 signaling or function. In some aspects, the PD-1 antagonist blocks binding of PD-1 to PD-L1, PD-L2, or to both PD-L1 and PD-L2. In some aspects, the PD-1 antagonist blocks binding of PD-1 to PD-L1. In some aspects, the PD-1 antagonist blocks binding of PD-1 to PD-L2. In some aspects, the PD-1 antagonist blocks the binding of PD-1 to PD-L1 and PD-L2. In some aspects, the PD-1 antagonist specifically binds PD-1. In some aspects, the PD-1 antagonist specifically binds PD-L1. In some aspects, the PD-1 antagonist specifically binds PD-L2.

In some aspects, the PD-1 antagonist inhibits the binding of PD-1 to its cognate ligand. In some aspects, the PD-1 antagonist inhibits the binding of PD-1 to PD-L1, PD-1 to PD-L2, or PD-1 to both PD-L1 and PD-L2. In some aspects, the PD-1 antagonist does not inhibit the binding of PD-1 to its cognate ligand.

In some aspects, the PD-1 antagonist is an isolated antibody (mAb), or antigen binding fragment thereof, which specifically binds to PD-1 or PD-L1. In some aspects, the PD-1 antagonist is an antibody or antigen binding fragment thereof that specifically binds to human PD-1. In some aspects, the PD-1 antagonist is an antibody or antigen binding fragment thereof that specifically binds to human PD-L1. In some aspects, the PD-1 antagonist is an antibody or antigen binding fragment that binds to human PD-L1 and inhibits the binding of PD-L1 to PD-1. In some aspects, the PD-1 antagonist is an antibody or antigen binding fragment that binds to human PD-1 and inhibits the binding of PD-L1 to PD-1.

Several immune checkpoint antagonists that inhibit or disrupt the interaction between PD-1 and either one or both of its ligands PD-L1 and PD-L2 are in clinical development or are currently available to clinicians for treating cancer.

Examples of anti-human PD-1 antibodies, or antigen binding fragments thereof, that may comprise the PD-1 antagonist in any of the compositions, methods, and uses provided by the disclosure include, but are not limited to: KEYTRUDA® (pembrolizumab, MK-3475, h409A11; see U.S. Pat. Nos. 8,952,136, 8,354,509, 8,900,587, and EP2170959, all of which are included herein by reference in their entirety; Merck), OPDIVO® (nivolumab, BMS-936558, MDX-1106, ONO-4538; see U.S. Pat. Nos. 7,595,048, 8,728,474, 9,073,994, 9,067,999, EP1537878, U.S. Pat. Nos. 8,008,449, 8,779,105, and EP2161336, all of which are included herein by reference in their entirety; Bristol Myers Squibb), MEDI0680 (AMP-514), BGB-A317 and BGB-108 (BeiGene), 244C8 and 388D4 (see WO2016106159, which is incorporated herein by reference in its entirety; Enumeral Biomedical), PDR001 (Novartis), and REGN2810 (Regeneron). Accordingly, in some aspects the PD-1 antagonist is pembrolizumab. In some aspects, the PD-1 antagonist is nivolumab.

Examples of anti-human PD-L1 antibodies, or antigen binding fragments thereof, that may comprise the PD-1 antagonist in any of the compositions, methods, and uses provided by the disclosure include, but are not limited to: BAVENCIO® (avelumab, MSB0010718C, see WO2013/79174, which is incorporated herein by reference in its entirety; Merck/Pfizer), IMFINZI® (durvalumab, MEDI4736), TECENTRIQ® (atezolizumab, MPDL3280A, RG7446; see WO2010/077634, which is incorporated herein by reference in its entirety; Roche), MDX-1105 (BMS-936559, 12A4; see U.S. Pat. No. 7,943,743 and WO2013/173223, both of which are incorporated herein by reference in their entirety; Medarex/BMS), and FAZ053 (Novartis). Accordingly, in some aspects the PD-1 antagonist is avelumab. In some aspects, the PD-1 antagonist is durvalumab. In some aspects, the PD-1 antagonist is atezolizumab.

In some aspects, the PD-1 antagonist is an immunoadhesin that specifically bind to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342, both of which are incorporated herein by reference in their entirety. In some aspects, the PD-1 antagonist is AMP-224 (also known as B7-DCIg), which is a PD-L2-FC fusion protein that specifically binds to human PD-1.

It will be understood by one of ordinary skill that any PD-1 antagonist which binds to PD-1 or PD-L1 and disrupts the PD-1/PD-L1 signaling pathway, is suitable for compositions, methods, and uses disclosed herein.

In some aspects, the PD-1/PD-L1 antagonist is a small molecule, a nucleic acid, a peptide, a peptide mimetic, a protein, a carbohydrate, a carbohydrate derivative, or a glycopolymer. Exemplary small molecule PD-1 inhibitors are described in Zhan et al., (2016) Drug Discov Today 21 (6): 1027-1036.

III.A.3. Combinations with TIM-3 Inhibitors

In some aspects, an anti-CD161 antibody, or antigen binding portion thereof, provided by the disclosure is combined (e.g., administered in combination) with a TIM-3 inhibitor. The TIM-3 inhibitor may be an antibody, an antigen binding fragment thereof, an immunoadhesin, a fusion protein, or an oligopeptide. In some aspects, the TIM-3 inhibitor is chosen from MGB453 (Novartis), TSR-022 (Tesaro), or LY3321367 (Eli Lilly). In some aspects, the anti-CD161 antibody, or antigen binding portion thereof, is administered in combination with MGB453. In some aspects, the anti-CD161 antibody, or antigen binding portion thereof, is administered in combination with TSR-022.

III.A.4. Combinations with LAG-3 Inhibitors

In some aspects, an anti-CD161 antibody, or antigen binding portion thereof, provided by the disclosure is combined (e.g., administered in combination) with a LAG-3 inhibitor. The LAG-3 inhibitor may be an antibody, an antigen binding fragment thereof, an immunoadhesin, a fusion protein, or oligopeptide. In some aspects, the LAG-3 inhibitor is chosen from LAG525 (Novartis), BMS-986016 (Bristol-Myers Squibb), TSR-033 (Tesaro), MK-4280 (Merck & Co), or REGN3767 (Regeneron).

### III.A.5. Other Combinations

In some aspects, an anti-CD161 antibody, or antigen binding portion thereof, provided by the disclosure is combined (e.g., administered in combination) with a TIGIT inhibitor, a kinase inhibitor (e.g., a tyrosine kinase inhibitor (TKI)), a CD112R inhibitor, a TAM receptor inhibitor, a STING agonist and/or a 4-1BB agonist, or a combination thereof. In some aspects, an anti-CD161 antibody, or antigen binding portion thereof, provided by the disclosure is combined (e.g., administered in combination) with a tyrosine kinase inhibitor, an agent targeting the adenosine axis (for example a CD39 antagonist, a CD73 antagonist or a A2AR, A2BR or dual A2AR/A2BR antagonist), a CCR8 antagonist, a CTLA4 antagonist, a VEG-F inhibitor or a combination thereof

### III.B. Methods of Engineering Antibodies

The anti-CD161 antibodies having VH and VL sequences disclosed herein can be used to create new anti-CD161 antibodies by modifying the VH and/or VL sequences, or the constant region(s) attached thereto. Thus, in another aspect described herein, the structural features of an anti-CD161 antibody described herein are used to create structurally related anti-CD161 antibodies that retain at least one functional property of the anti-CD161 antibodies described herein, such as inhibiting the interaction between human CD161 and CLEC2D. For example, one or more CDR regions of the antibodies disclosed herein can be combined recombinantly with known framework regions and/or other CDRs to create additional, recombinantly-engineered, anti-CD161 antibodies described herein, as discussed above. The starting material for the engineering method is one or more of the VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

In addition, the antibodies disclosed herein can be improved through known techniques such as affinity maturation. Affinity maturation is a technique which allows for the selection of derivative antibodies that bind to an antigen with greater affinity than a starting antibody. In some aspects, the antibodies disclosed herein are used as the starting antibody for affinity maturation.

Accordingly, provided herein are methods for preparing an anti-CD161 antibody described herein.

### III.C. Antibody Production

Anti-CD161 antibodies described herein can be produced using a variety of known techniques, such as the standard somatic cell hybridization technique described by Kohler and Milstein, Nature 256:495 (1975). Although somatic cell hybridization procedures are common, in principle, other techniques for producing monoclonal antibodies also can be employed, e.g., viral or oncogenic transformation of B lymphocytes, phage display technique using libraries of human antibody genes.

In some aspects, the animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized anti-CD161 antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see, e.g., U.S. Pat. No. 4,816,567 to Cabilly et al.). To create a humanized antibody, the murine CDR regions can be inserted into a human framework using methods known in the art (see, e.g., U.S. Pat. No. 5,225,539 to Winter, and U.S. Pat. Nos. 5,530,101; 5,585,089; 5,693,762 and 6,180,370 to Queen et al).

In some aspects, the anti-CD161 antibodies described herein are human monoclonal antibodies. Such human monoclonal antibodies directed against CD161 can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HUMAB-MOUSE® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see, e.g., Lonberg, et al., (1994) Nature 368 (6474): 856-859). Accordingly, the mice exhibit reduced expression of mouse IgM or κ, and in response to immunization, the introduced human heavy and light chain transgenes undergo class switching and somatic mutation to generate high affinity human IgGK monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13:65-93, and Harding, F. and Lonberg, N. (1995) Ann. N.Y. Acad. Sci. 764:536-546).

In some aspects, the anti-CD161 antibodies described herein are raised using a mouse that carries human immunoglobulin sequences on transgenes and transchomosomes, such as a mouse that carries a human heavy chain transgene and a human light chain transchromosome. Such mice, referred to herein as “KM mice,” are described in detail in PCT Publication WO 02/43478 to Ishida et al.

Still further, alternative transgenic animal systems expressing human immunoglobulin genes are available that can be used to raise anti-CD161 antibodies described herein. For example, an alternative transgenic system referred to as the Xenomouse (Abgenix, Inc.) can be used; such mice are described in, for example, U.S. Pat. No. 5,939,598.

Moreover, alternative transchromosomic animal systems expressing human immunoglobulin genes are available in the art and can be used to raise anti-CD161 antibodies described herein. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-CD161 antibodies described herein.

Additional mouse systems described in the art for raising human antibodies, e.g., human anti-CD161 antibodies, include (i) the VELOCLMMUNE® mouse (Regeneron Pharmaceuticals, Inc.), in which the endogenous mouse heavy and light chain variable regions have been replaced, via homologous recombination, with human heavy and light chain variable regions, operatively linked to the endogenous mouse constant regions, such that chimeric antibodies (human V/mouse C) are raised in the mice, and then subsequently converted to fully human antibodies using standard recombinant DNA techniques; and (ii) the MEMOR mouse (Merus Biopharmaceuticals, Inc.), in which the mouse contains unrearranged human heavy chain variable regions but a single rearranged human common light chain variable region. Such mice, and use thereof to raise antibodies, are described in, for example, US 2012/0070861 and US 2012/0073004.

Human monoclonal anti-CD161 antibodies described herein can also be prepared using phage display methods for screening libraries of human immunoglobulin genes. Such phage display methods for isolating human antibodies are established in the art. See for example: U.S. Pat. Nos. 5,223,409; 5,427,908; 5,969,108; and 5,885,793.

Human monoclonal anti-CD161 antibodies described herein can also be prepared using SCID mice into which human immune cells have been reconstituted such that a human antibody response can be generated upon immunization. Such mice are described in, for example, U.S. Pat. No. 5,476,996.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooks, Antisense drug Technology: Principles, strategies and applications, 2nd Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Example 1

### Generation of Anti-CD161 Monoclonal Antibodies

Human Ig transgenic mice were used to generate anti-CD161 monoclonal antibodies. A cohort of eight mice were immunized with human CD161-hFc protein from AcroBiosystems (CD1-H5253), along with DNA immunogens expressing human and cyno CD161 ECD domains which included TCE-huCD161-ECD-67-225 and TCE-cyCD161-ECD-67-225. CD161 positive antibody titers were checked on Day 21 and Day 28 post immunizations by flow cytometry using human and cyno CD161 overexpressing cell-lines including CHOK1: huCD161, CHOK1: cyCD161, and CHOK1: Parental cells. The eight mice received four DNA and eight protein immunizations prior to the Day 28 titer check. Day 28 plasma titer analysis indicated strong CD161 positive antibody titers in all the eight immunized mice.

### Hybridoma Generation and Antibody Screening Assays

Spleen and lymph nodes were harvested from the eight immunized mice on Day 32 post immunization regimen with CD161 protein and DNA to generate hybridomas. Hybridoma fusion with enriched B cells from spleen and lymph nodes was performed and plated out in fourteen 384 well plates. After eight days primary multiplex fluorescence-activated cell sorting (FACS) binding screening was performed using CHOK1: huCD161, CHOK1: cyCD161, CHOK1: P cells (cell mix) to isolate CD161 positive binding hits. For binding screening hybridoma, supernatants of 15 μL/well were transferred to a 384-well plate containing 15 μL of 20,000/well cell mix. The plate was incubated for 30 minutes (min) at 4° C. and then washed 2.5× with FACS buffer. The cells were then stained with 15 μL/well of 1 μg/mL AF647-Goat αHuman-Fc and incubated for 30 min at 4° C. Subsequently, the cells were washed 2.5× with FACS buffer before reading on the flow cytometer. Anti-CD161 reference monoclonal antibody HP-3G10 (Biolegend; Cat #39910) was used as a positive control.

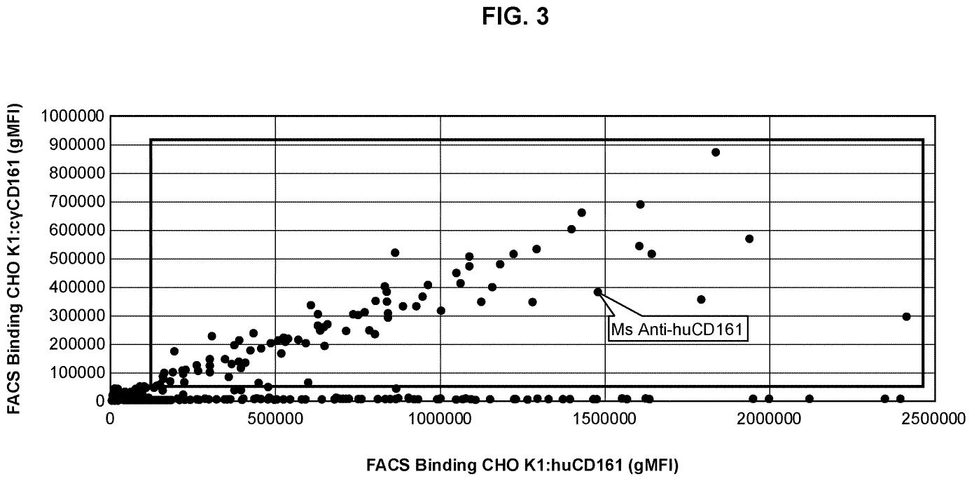

From the primary FACS based screening, 134 binders to human and cyno CD161 were identified. These hybridomas that bound to HEK293T: huCD161 with >50K geometric mean fluorescence intensity (gMFI) and >30K gMFI to HEK293T: cyCD161 were selected for further validation as shown in FIG. 3. These CD161 positive binders were then evaluated for their blocking or inhibiting of the CD161-CLEC2D interaction. Of the 134 CD161 positive binders, 34 hybridomas that demonstrated effective blocking of the interaction of CD161 with its ligand, CLEC2D, were selected for subcloning.

Screening Assays: Cell-based Binding Assay with CD161 Antibodies

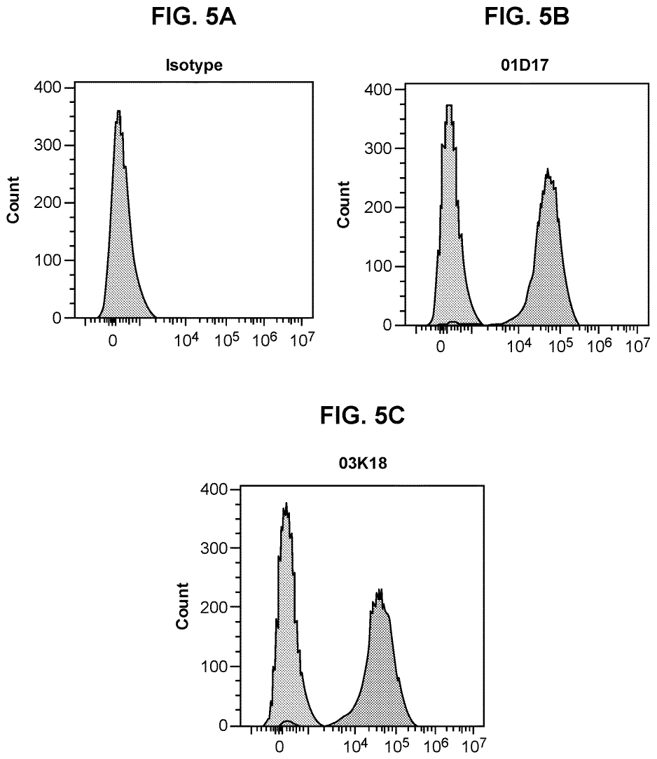

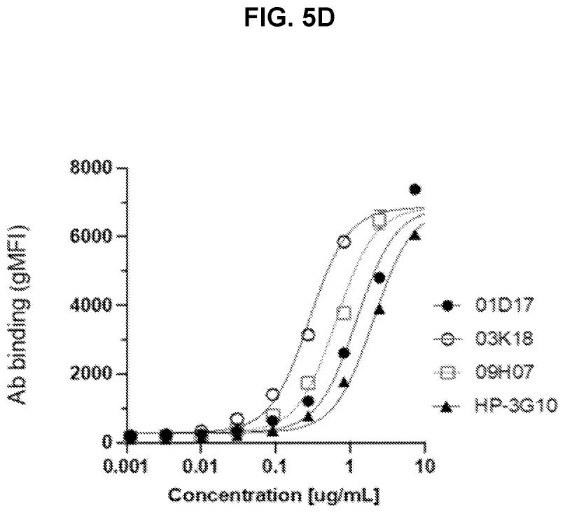

Cell based binding assays were performed with the CD161 antibodies to assess the cell surface binding capacity of anti-CD161 antibodies with huCD161 antigen expressed on CHOK1 cell surface using FACS. CHOK1 suspension cells expressing full-length membrane anchored huCD161 antigen were used to screen hybridoma antibody samples. The 50,000 cells were washed and transferred to 96 well round bottom plate followed by adding 50 μl of purified antibody (0.001-10 μg/ml in Dulbecco's phosphate-buffered saline (DPBS)) and incubated for 30 min at 4° C. After incubation, the plate was centrifuged at 300×g for 5 min. The cells were further washed twice with FACS buffer (1% FBS in DPBS) and were stained with goat α-Mouse IgG, Fcγ AF647 (1:2000 dilution). The plate was incubated for 20 min at 4° C. in the dark. After incubation, the plate was centrifuged at 300×g for 5 min. The cells were further washed twice with FACS buffer and suspended again in 100 μl FACS buffer for FACS analysis. Commercially available anti-CD161 monoclonal Ab (HP-3G10, Biolegend) was used as a positive control and mouse IgG1 isotype (MOCP1, BioXcell) as the negative control. Anti-CD161 antibodies generated from the hybridomas can bind with huCD161 antigen expressed on CHOK1 cell surface as shown in FIGS. 5A-5D. Representative antibody clones, as exemplified by 01D17, 03K18, 09H07, and HP-3G10 (control), were evaluated for binding on CHOK1 transfected with the full length huCD161 construct as shown in FIGS. 5A-5C. Full-dose response curves are shown in FIG. 5D.

Screening Assays: ELISA-based Binding Assay with CD161 Antibodies

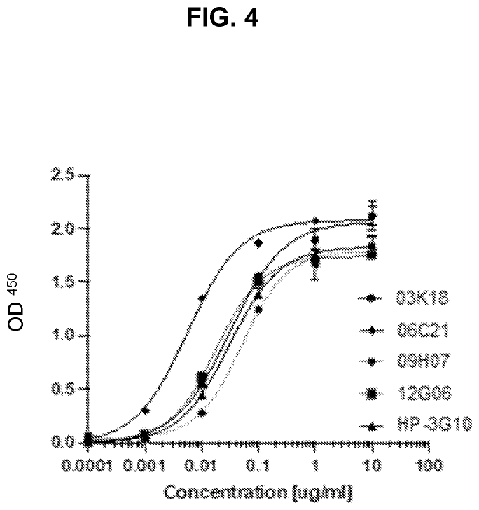

The binding interaction of purified anti-CD161 antibodies from hybridoma supernatants with immobilized rhCD161-hFc was evaluated by ELISA. A high-binding 96 well ELISA plate was coated with 100 μl of 5 μg/mL rhCD161-hFc protein (R&D) in DPBS overnight at 4° C. The plate was washed 3× with ELISA wash buffer (Biolegend) then blocked with 200 μl DPBS with 5% milk. The plate was then incubated at 37° C. on a gentle shaker at 120 rpm for 2 hours (hrs) then washed 3× with ELISA wash buffer. Purified antibody of 0.0001-10 μg/ml in 100 μl assay buffer (1% BSA in DPBS) was added to each well and incubated for 2 hrs at 37° C. on gentle shaker at 120 rpm then subsequently washed 3× with ELISA wash buffer. Goat anti-mouse detection antibody HRP (1:3000) in 100 μl/well assay buffer was added and incubated for 1 hour (h) at 37° C. on gentle shaker at 120 rpm then washed 5× with ELISA wash buffer. Then, 100 μl TMB substrate was added per well and incubated at RT for 5 min. Following, 100 μl TMB stop solution was added to stop the reaction. Optical density (OD) of each well of the ELISA plate was read with a microplate reader set to the wavelength of 450 nm as illustrated in FIG. 4. Commercially available anti-CD161 monoclonal antibody (mAb) (HP-3G10, Biolegend) was used as a positive control and mouse IgG1 isotype (MOPC1, BioXcell) was used as the negative control. Anti-CD161 antibodies can bind with immobilized rhCD161-hFc as depicted with positive OD values shown in FIG. 4. Representative anti-CD161 antibody hybridoma clones including 03K18, 06C21, 09H07, 12G06, and HP-3G10 (control), were evaluated for binding with plate coated rhCD161 by ELISA.

### Screening Assays: Cell-based Target and Ligand Blocking Assay

The affinity of CLEC2D for CD161 as reported by Kamishikiryo, et al (2011), is a K_{D} of 48 μM. The use of the antibodies to block the CD161-CLEC2D interaction was evaluated. The capability of the anti-CD161 positive hybridoma hits to block the CD161-CLEC2D interaction was assessed using huCLEC2D soluble protein and CHOK1 cells expressing huCD161. Based on the lower affinity between CD161-CLEC2D, multimeric complex of CLEC2D protein was needed to be generated to increase the valency.

For generation of CLEC2D multimers, biotinylated-Protein A (ThermoFisher) and hCLEC2D-hFc protein (Kactus) were mixed at 1:20 ratio and incubated for 5 min at 4° C. in the dark. PBS was added to the mixture and incubated for 30 min at 4° C. in the dark. After incubation, Streptavidin-APC beads (Biolegend) were added (1:1000 dilution in DPBS) and incubated for 30 min at 4° C. in dark. Purified human IgG (ThermoFisher) was added for Fc saturation.

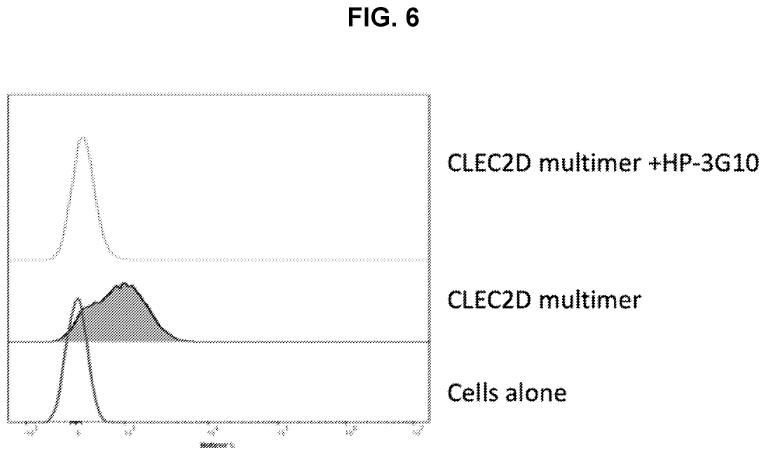

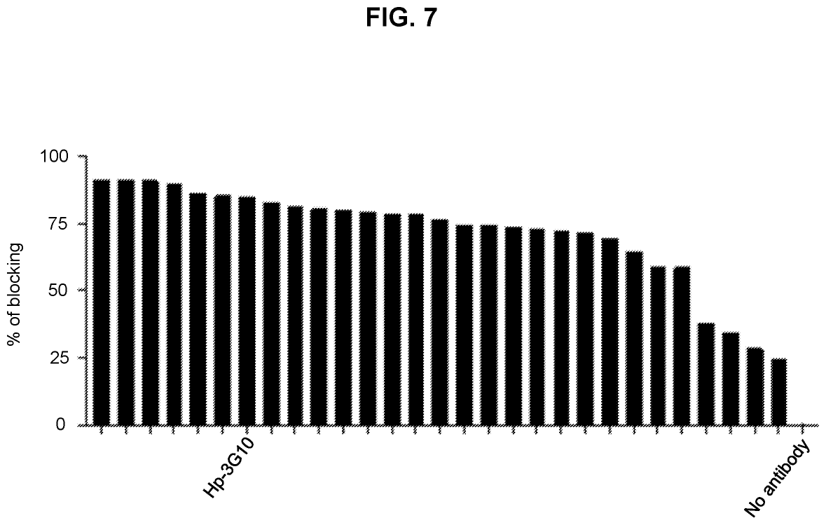

CHOK1 cells expressing huCD161 antigen were used to screen anti-CD161 antibody samples. Between 50,000 and 100,000 cells were washed and transferred to 96 well round bottom plate. Pre-prepared hCLEC2D multimer complex (20 μl) plus 50 μl of purified CD161 antibody sample was added to the CD161 expression cells and incubated for 30 min at 4° C. in the dark. After incubation, the plate was centrifuged at 300×g for 5 min. The cells were further washed twice with FACS buffer and suspended again in 100 μl FACS buffer for FACS analysis. Commercially available anti-CD161 mAb (HP-3G10, Biolegend) was used as a positive control and mouse IgG1 isotype (MOPC1, BioXcell) was used as the negative control. The percentage of blocking CD161-CLEC2D interaction was calculated using the formula: % of blocking=(1-% CLEC2D multimer bound to CHOK1-CD161 with antibody/% CLEC2D multimer bound to CHOK1-CD161 without antibody)×100%. As shown in FIG. 6, hCLEC2D multimer effectively binds to CHOK1 expressing huCD161. Reference anti-CD161 monoclonal Ab (HP-3G10) at 10 μg/ml resulted in full blocking of hCLEC2D multimer (FIG. 6). The percentage of blocking of anti-CD161 antibodies able to block the interaction between the CLEC2D multimer and huCD161 expressed on the cell surface are shown in FIG. 7. Anti-CD161 antibodies from the selected hybridomas were assessed by FACS and CLEC2D multimer bound to CHOK1 expressing CD161. The percentage of blocking was ranked based on blocking of the interaction between hCLEC2D multimer and hCD161.

### Screening Assays: ELISA-based CD161: CLEC2D Blocking Assay

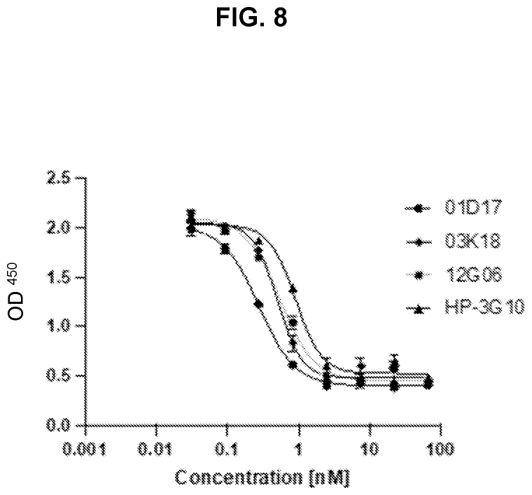

The capacity of anti-CD161 antibodies to block the interaction between rhCLEC2D and immobilized rhCD161-hFc was evaluated by ELISA. A high-binding 96-well ELISA plate was coated with 100 μl rhCD161-hFc (5 μg/ml) protein (R&D) in DPBS overnight at 4° C. The plate was washed 3× with ELISA wash buffer (Biolegend) then blocked with 200 μl DPBS with 5% milk and incubated at 37° C. on gentle shaker at 120 rpm for 2 hrs then washed 3× with ELISA wash buffer. Biotinylated 10 μg/ml CLEC2D-hFc (Kactus) and 0.03-66.7 nM of purified antibody in 100 μl assay buffer (1% BSA in DPBS) was added to each well and incubated for 2 hrs at 37° C. on gentle shaker at 120 rpm then subsequently washed 3× with ELISA wash buffer. Avidin HRP (1:1000) in 100 μl assay buffer was added to each well of the plate and incubated for 1 h at 37° C. on gentle shaker at 120 rpm then washed 5× with ELISA wash buffer. A volume of 100 μl TMB substrate was added to each well and incubated at 37° C. for 30 min and 100 μl TMB stop solution was added to stop the reaction. Optical density (OD) of each well of the ELISA plate was read with a microplate reader set to the wavelength of 450 nm. Commercially available anti-CD161 mAb (HP-3G10, Biolegend) was used as a positive control and mouse IgG1 isotype (MOPC1, BioXcell) as the negative control. As illustrated in FIG. 8, anti-CD161 antibodies can block the interaction between rhCLEC2D and immobilized rhCD161-hFc. Representative anti-CD161 hybridoma clones, including 01D17, 03K18, 12G06, and HP-3G10 (positive control), were evaluated for competing with CLEC2D-CD161 contact sites and disrupting the interaction.

### Example 2. Functional Assessment of Anti-CD161 Monoclonal Antibodies

T Cell Activity Assessment with Staphylococcal Enterotoxin B Stimulation

Peripheral blood mononuclear cells (PBMC) from healthy human donors were stimulated with 10 ng/ml Staphylococcal enterotoxin B (SEB) with or without test anti-CD161 antibody for 24 hrs in complete cell culture media (RPMI+10% FBS) at 37° C., followed by measurement of IL-2 cytokine in cell culture supernatants. Commercially available anti-CD161 monoclonal antibody (mAb) (HP-3G10, Biolegend) was used as a positive control, and mouse IgG1 isotype (MOPC1, BioXcell) was used as the negative control. The percent increase of IL-2 cytokine was calculated using the following formula: % of increase=(cytokine concentration of sample with antibody/cytokine concentration of sample without antibody)×100%-100%.

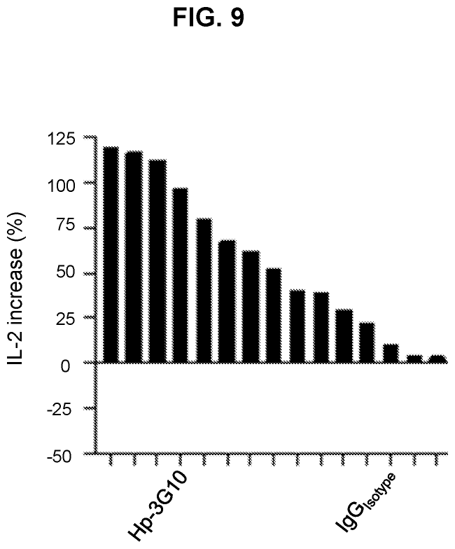

Anti-CD161 antibodies that can enhance T cells function as measured by IL-2 cytokine release by blocking the CD161-CLEC2D interaction is shown in FIG. 9, a representative ELISA analysis for IL-2 cytokine expression from PBMC supernatants of one donor. Anti-CD161 antibodies were ranked based on IL-2 release level compared to without test antibody added in the SEB stimulated PBMC as percent increase of IL-2 (FIG. 9).

### T Cell Activity Mediated by T Cell Engager

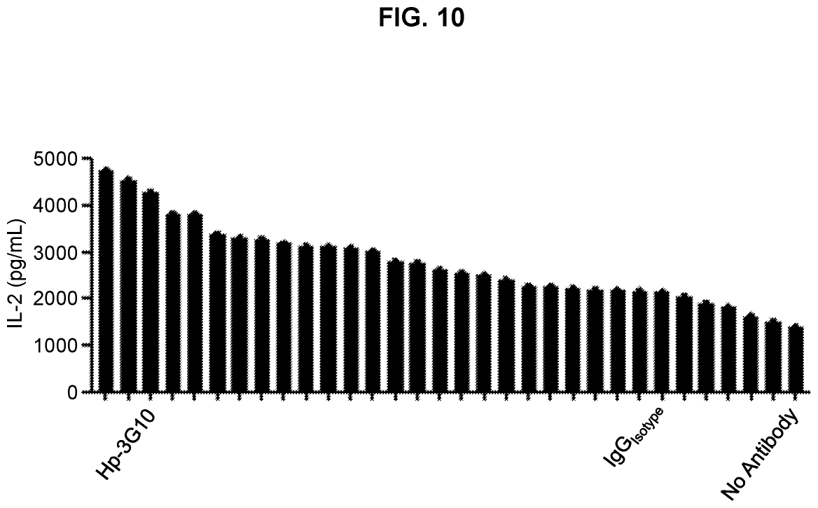

An ELISA assay was used to analyze IL-2 cytokine level in PBMC supernatant, and anti-CD161 antibodies were assessed and ranked based on IL-2 release. For this assay, Raji cells were washed and transferred to a 96-well round bottom plate at 30,000 cells per well and were pre-incubated with 100 μg/ml CD3×CD19 T cell engager (InvivoGen) for 30 min at 37° C. An amount of 90,000 Jurkat cells expressing full-length membrane-anchored huCD161 antigen together with purified anti-CD161 antibody and human Fc blocker (1:1000) were added to the Raji/T cell engager plate. The plate was incubated for 48 h at 37° C., followed by measurement of cytokine in cell culture supernatants. Commercially available anti-CD161 mAb (HP-3G10, Biolegend) was used as a positive control and mouse IgG1 isotype (MOPC1, BioXcell) was used as the negative control. As illustrated in FIG. 10, anti-CD161 antibodies can enhance Jurkat cells expressing huCD161 function, measured by IL-2 release, by blocking hCD161-hCLEC2D interaction.

### Example 3. Anti-CD161 Monoclonal Antibodies Enhance Activation of T Cells when Inhibiting the CD161-CLEC2D Interaction

The anti-CD161 monoclonal antibodies (mAbs) were assayed for the ability to rescue the activation of MART-1 TCR+hCD161+Jurkat cells by hCLEC2D overexpressing MeWo cells (HLA-A2+) in the presence of MART-1 peptide (HLA-A2-restricted).

### Methods

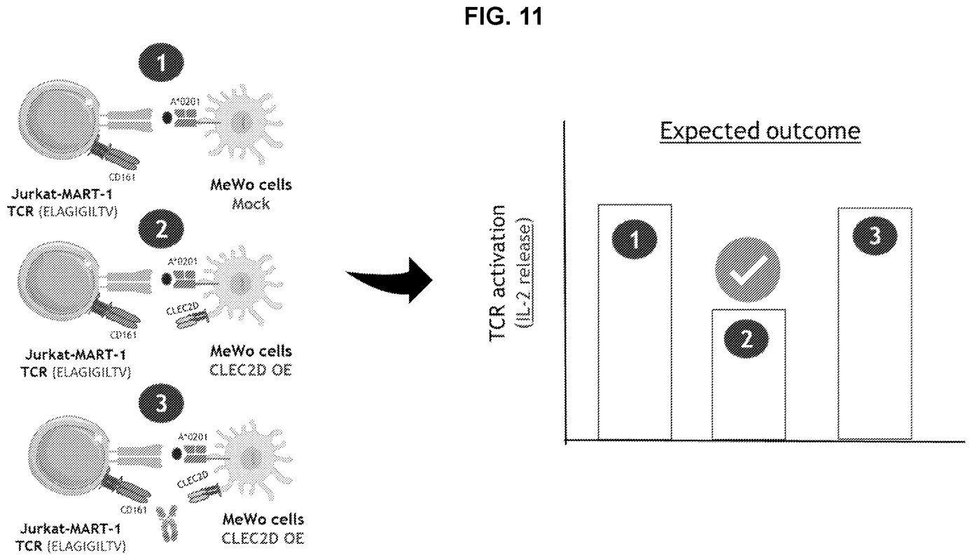

Mock-GFP and hCLEC2D-GFP over expressing MeWo cell line (HLA; A*0201, which is needed for peptide presentation) were cultured overnight at 20K/well in FB 96 well plate (see FIG. 11). On the next day, the plate was centrifuged at 300 g/5 min, the medium was carefully removed without touching adherent MeWo cells, and then 0.1 ug/well of MART-1 peptide prepared in RPMI complete medium was added and the cells were incubated for 30 min/37° C. During incubation, a round bottom plate 50 μL of Jurkat-MART-1 TCR with human CD161 overexpressing cells (100K/well) was prepared, and then 50 μL of anti-CD161 mAbs was added at a final concentration from 0.005 to 10 μg/mL. After peptide plate incubation, the Jurkat-MART1-CD161 OE cell line with anti-CD161 mAbs 100 μL was added on top of the MeWo cells with peptide FB in a 96-well plate and incubated for 24 hr at 37° C. On the next day, after incubation, the plate was centrifuged at 500 g for 5 minutes, and 180 μL of the supernatant was collected for IL-2 ELISA, using a Biolegend Kit (CAT #431816 LOT: B352552; as per manufacturer instruction).

### Results

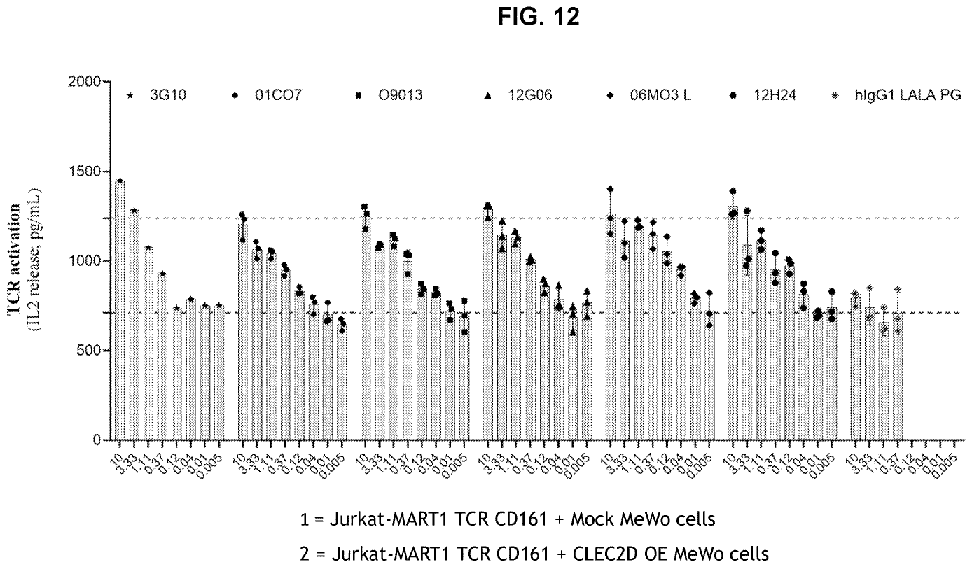

Each anti-CD161 mAbs elicited a dose-dependent increase in TCR activation, as measured by IL-2 secretion compared to an isotype control (FIG. 12). These results indicate that TCR activation is influenced by human CLEC2D, and it can be rescued with an anti-CD161 antibody, which blocks the interaction between CD161-receptor (Jurkat) and its ligand CLEC2D expressed on MeWo cells. As expected, the control hIgG1 LALA-PG antibody did not increase further TCR activation.

The anti-CD161 antibodies thus inhibit the negative interaction between CD161 and CLEC2D and enhance T cell activation, as measured by IL-2 release.

### Example 4. Anti-CD161 Monoclonal Antibodies Bind to TALL-104 Cells and Rescue the Cytolysis of hCLEC2D Overexpressing Target Cells by TALL-104 Cells

The anti-CD161 mAbs were assayed for the ability to bind TALL-104 effector cells and to rescue the cytolysis of hCLEC2D overexpressing (OE) PC3 target cells by CD161+TALL-104 as effector cells (CD3/TCR+CD4-CD8+CD56+CD16−). TALL-104 effector cells are a clinically relevant MHC non-restricted human cytotoxic T cell line.

### TALL-104 Binding

TALL-104 cells were washed and counted, then placed in a 96-well round bottom plate at 100,000 cells/well. The plate was centrifuged at 500 g for 5 minutes, and the supernatant discarded. Next, 50 μL of anti-CD161 mAbs prepared in FACS buffer was added, the samples were mixed, and then incubated on ice for 30 minutes. After incubation, the plate was washed with 100 μL FACS buffer (DPBS+1% FBS), centrifuged at 500 g for 5 minutes, and the supernatant was discarded. The plates were then incubated with 50 μL of goat anti-Human IgG (H+L) cross-adsorbed secondary antibody, ALEXA FLUOR™ Plus 647 with zombie aqua, and a 1:2000 diluted live dead dye, for 20 minutes on ice. After incubation, the plate was washed with 100 μL of FACS buffer (DPBS+1% FBS). The plate was then centrifuged at 500 g for 5 minutes. Then the supernatant was discarded and the cell pellet was resuspended in 100 μL/well FACS buffer before acquiring with a flow cytometer.

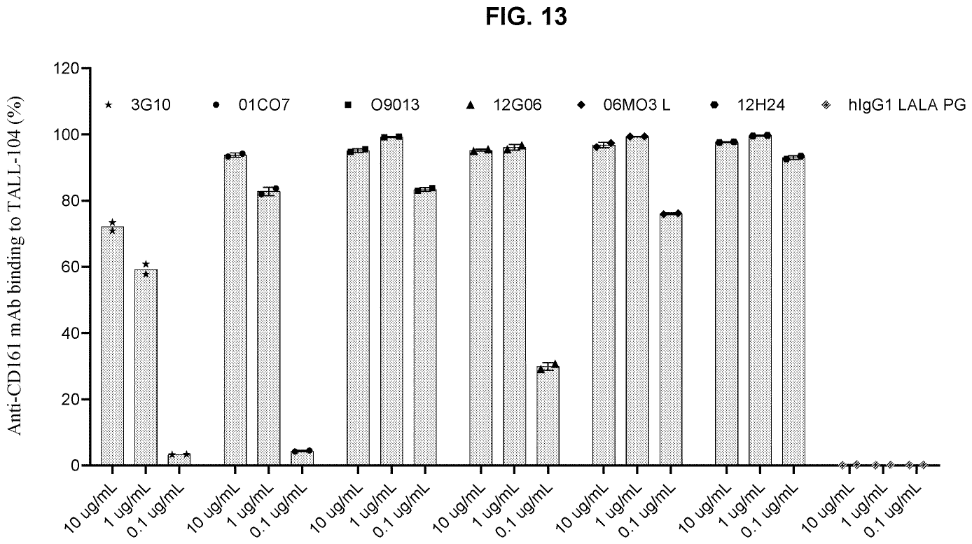

Each of the anti-CD161 mAbs tested was capable of binding TALL-104 cells, in a dose-dependent manner (FIG. 13).

### Rescue of TALL-104 Effector Function

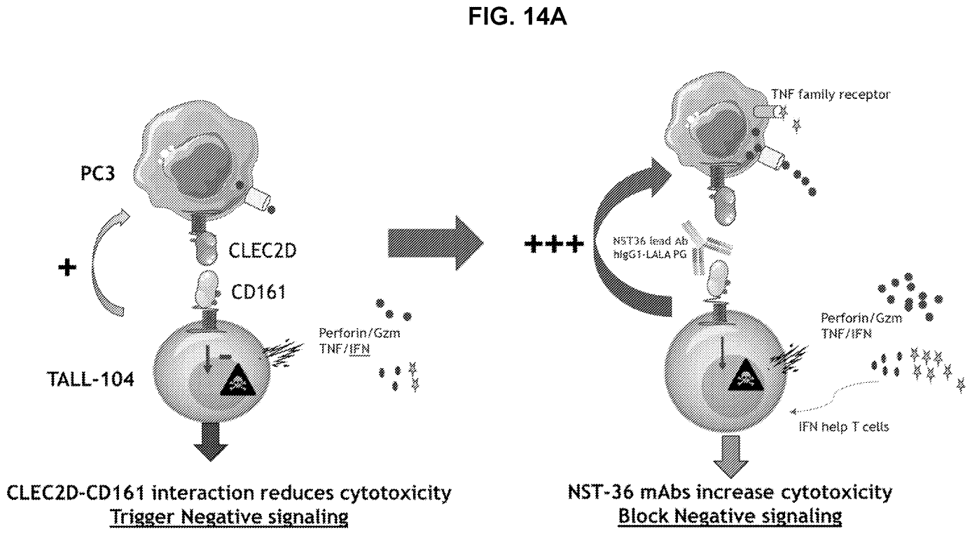

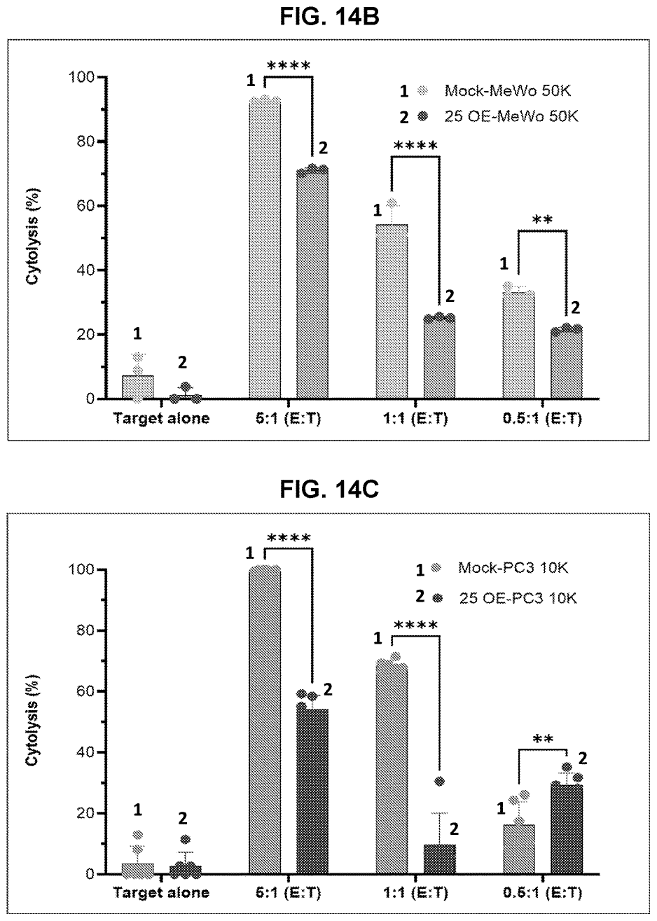

The xCELLigence-based cytotoxicity assay was used to assess the ability of the anti-CD161 mAbs to rescue the cytolysis of hCLEC2D overexpressing PC3 target cells by CD161+TALL-104 effector cells (CD3/TCR+CD4-CD8+CD56+CD16−) (FIG. 14A). CD161+TALL-104 human killer CD8+ T cells were incubated with CLEC2D+tumor cells (PC3-CLEC2D OE). Overexpression of CLEC2D on PC3 tumor cells inhibits TALL-104 mediated killing as compared to WT PC3 cells (CLEC2D negative) (FIGS. 14B-14C). The anti-CD161 mAbs that were shown above to block CD161-CLEC2D interactions were tested for their ability to enhance the TALL-104 mediated killing of PC3-CLEC2D OE.

### Step 1: Adherent Target Cells (i.e., Tumor Cells—PC3)

On day-1, Mock/OE PC-3 cells were trypsinized and counted. Cell were then plated overnight at 10K cells/well in a 96-well E-plate.

### Step 2: TALL-104 Addition as Effector Cells (i.e., Immune Cells)

The next day, TALL-104 cells were counted and spun down. The TALL-104 cells were then resuspended in media and treated with the anti-CD161 mAbs or the isotype IgG1 LALA-PG control for 30 min/37° C. After incubation, the TALL-104 cells were contacted with PC3 cells at a 1:1 ratio in an E-Plate and incubated at RT for 30 minutes to facilitate uniform distribution of TALL-104 cells on top of the PC3 target cells. The plates were then placed back into xCELLigence instrument located inside incubator, and data was acquired for 20 hr at 37° C.

Step 3: If Effector Cells Induce the Destruction of the Target Adherent Tumor Cells, this Cytolytic Activity can be Sensitively and Precisely Detected

The xCELLigence RTCA label-free technology counts cells using impedance changes in gold electrodes embedded in proprietary E-Plates. In this xCELLigence assay, hCLEC2D OE PC3 tumor cells are adhered to the surface of interdigitated gold microelectrodes that are embedded in the bottoms of microtiter plates (E-Plates). The interaction of target cells with gold sensors produces an impedance signal that reflects the target cells' number, size, and attachment strength. The fact that the effector TALL-104 cells are not naturally adherent simplifies this system because they generate a low impedance signal that can be easily subtracted. However, when TALL-104 attacks and kills PC3 cells via cytolysis, the viability of the target PC3 cells is reflected by real-time changes in impedance, providing a kinetic assessment of killing. TALL-104's cytolytic activity causes adherent cells to round up and detach, leading to a lower CI value

The RTCA system uses cellular impedance readout to monitor real-time changes in cell number, cell size, and cell-substrate attachment strength as a single parameter called Cell Index (CI) to reflect the viability of target PC3 cells. To demonstrate the effectiveness of this approach as a potency assay for TALL-104 cell-mediated cytolysis of target PC3 cells. First, PC3 prostate cancer cells, engineered to be either mock as a control (Mock-PC3) or human CLEC2D overexpressing cells (CLEC2D OE PC3), were cultured in E-Plates and their proliferation was measured using xCELLigence. The day after seeding the PC3 target cells, TALL-104 effector cells were added to the wells at an E:T ratio of 1:1, and the target PC3 cells' viability was monitored. PC3 cells treated with effector cell growth media alone served as a negative control. The addition of TALL-104 cells to PC3 cells resulted in an immediate and time-dependent decrease in CI. The PC3 signal dropped approximately 24 hours after the addition of effector cells due to cytolysis.

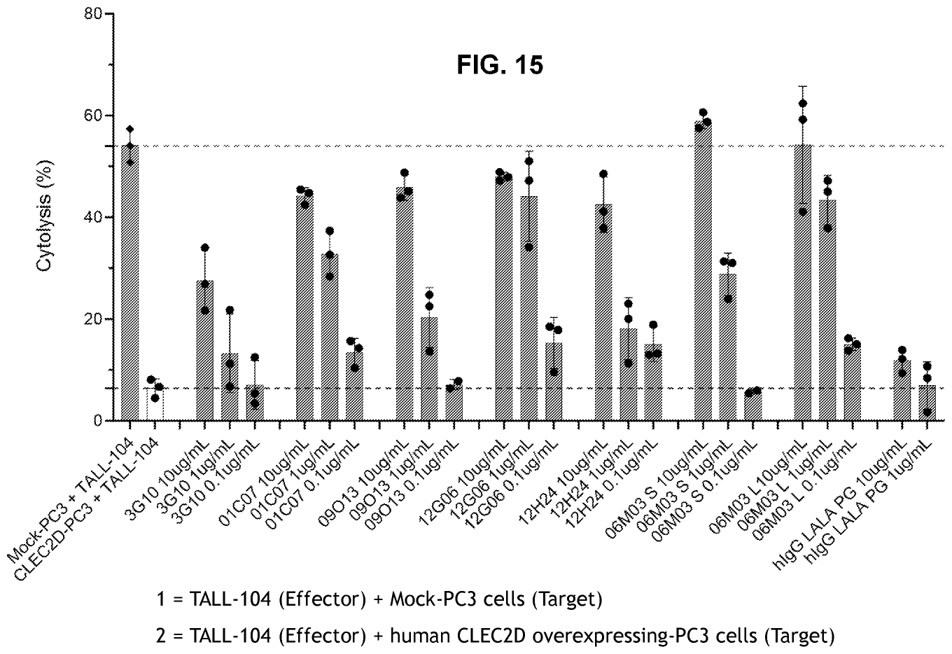

Each anti-CD161 mAbs showed a dose dependent increase in cytolysis only in human CLEC2D OE-PC3 cells (FIG. 15), indicating that TALL-104-based PC3 killing is influenced by human CLEC2D and can be rescued with an anti-CD161 antibody that blocks the interaction between CD161-receptor (TALL-104) and its ligand CLEC2D express on PC3 cells.

### Example 5. Anti-CD161 mAbs Increase NK Cytotoxicity

The anti-CD161 mAbs were assayed for effects on primary human NK cell cytotoxicity. Human NK cells were activated overnight with 200 U/mL IL-2, then co-cultured with cell trace violet (CTV) labeled PC-3 CLEC2D-OE (overexpressing CLEC2D) or Raji cells for 18 hours in the presence of Fc block plus either 1 μg/mL of an anti-CD161 mAb or an hlgG1-LALAPG control antibody. Cytotoxicity on tumor cells was measured by tumor cell viability (Zombie+) within the CTV+ population.

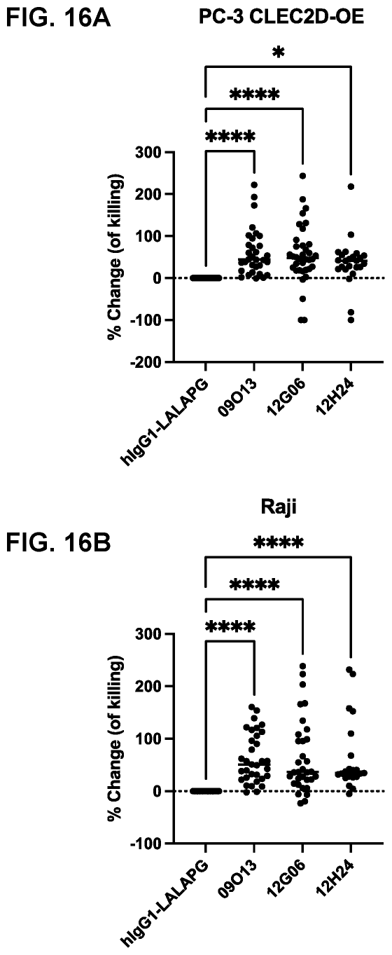

Contact with the anti-CD161 mAbs resulted in increased NK cytotoxicity of PC-3 CLEC2D-OE (FIG. 16A) or Raji (FIG. 16B) cells compared to isotype control, as assessed via relative tumor killing capacity. Relative tumor killing capacity was calculated by normalizing tumor cell death (%) in the presence of the anti-CD161 mAb to isotype. Data is representative of nine healthy donors for PC-3 CLEC2D-OE (FIG. 16A) and seven healthy donors for Raji (FIG. 16B) across 4 or 3 independent assays for each anti-CD161 clone.

### Example 6. Anti-CD161 mAbs Enhance Inhibition of Tumor Growth by NK Cells

Healthy naive female mice (6-8 weeks; NOD.Cg-Prkdc^{scid}II2^{tm1Wjl}/SzJ NSG; Jackson Laboratory) were subcutaneously injected with 2×10⁶ PC3-CLEC2D-OE cells. Mice were randomized to treatment groups (n=4) when tumor size reached ~70 mm³ (Day 4). The first two doses of the anti-CD161 antibody 12G06 (10 mg/kg) were injected intravenously, and the following six doses (15 mg/kg) were injected intraperitoneally. Treatment started on Day 4 and repeated every 3 days.

All mice received two administrations of 5×10⁶ human NK cells intratumorally on Day 4 and Day 10. NK cells were freshly isolated from frozen human PBMC and cultured with recombinant human IL-2 100U/mL in NK MACS media (Miltenyi Biotech) the night before injection.

Tumor growth was measured with calipers in two dimensions, and tumor volume (mm³) was calculated using the formula (width²×length)/2.

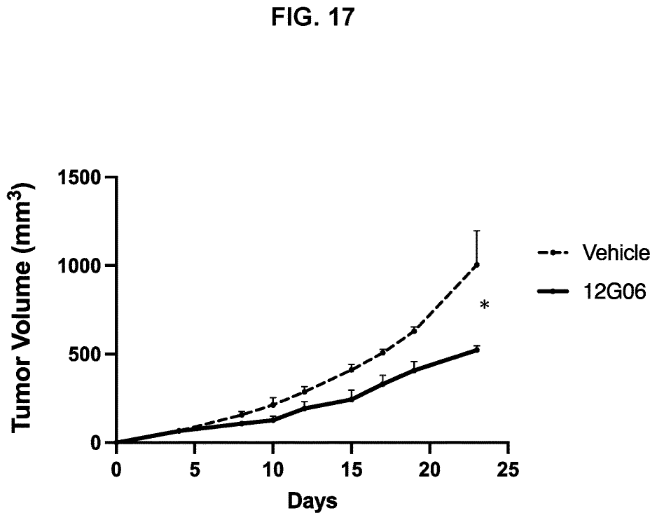

Administration of the 12G06 antibody significantly enhanced the tumor growth inhibition in the presence of NK cells, as compared to a vehicle control group, in the xenograft model (FIG. 17). These experiments will be repeated with additional anti-CD161 antibodies (e.g., 01C07, 09013, 12H24, 06M03 S, and/or 06M03 L).

### Example 7. Anti-CD161 mAbs Enhance Inhibition of Tumor Growth by Co-Implanted PBMCs

Healthy mice (NOD.Cg-Prkdc^{scid}II2^{tm1Wjl}/SzJ NSG; Jackson Laboratory) were subcutaneously injected into the right flank with 4.5×10⁶ cells of a 1:4 mixture of PBMCs and U-87 MG (glioblastoma) human tumor cells, the anti-CD161 antibody 12G06 was administered intraperitoneally 1 hour after implantation, and the mice received a total of eight doses of 20 mg/kg 12G06 every 3 days.

Tumor growth was measured with calipers in two dimensions, and tumor volume (mm³) was calculated using the formula (width²× length)/2.

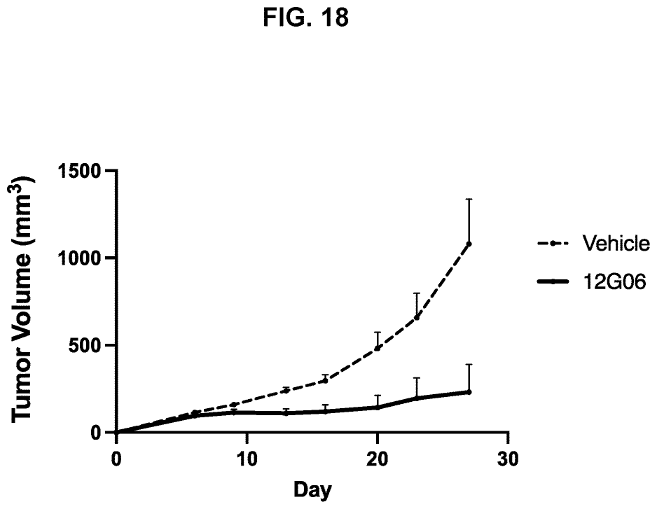

Administration of the 12G06 antibody significantly enhanced the tumor growth inhibition in the presence of human PBMC, as compared to a vehicle control group, in xenograft model (FIG. 18). These experiments will be repeated with additional anti-CD161 antibodies (e.g., 01C07, 09013, 12H24, 06M03 S, and/or 06M03 L).

### Example 8. In Vivo Analysis of Anti-CD161 Antibodies in Human Cancer Patients (Prophetic)

The safety and efficacy of the anti-CD161 antibodies will be tested in human cancer patients. Patients will be administered a dose of the 12G06, 01C07, 09013, 12H24, 06M03 S, and/or 06M03 L antibody. Patients will be monitored for any adverse events, and tumor burden, progression free survival, and overall survival will be determined.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, U.S. or foreign patents or patent applications, and websites) that are cited throughout this application are hereby expressly incorporated by reference as if written herein in their entireties for any purpose, as are the references cited therein. Where any inconsistencies arise, material literally disclosed herein controls.

What is claimed is:

1. An antibody or an antigen-binding portion thereof that specifically binds CD161, comprising a heavy chain variable region (VH) and a light chain variable region (VL); wherein the VH comprises a VH complementarity determining region 1 (VH-CDR1), a VH-CDR2, and a VH-CDR3;
wherein the VL comprises a VL-CDR1, a VL-CDR2, and a VL-CDR3; and
wherein the VH-CDR3 comprises an amino acid sequence selected from the sequences set forth in SEQ ID NOs: 3, 13, 23, 33, 43, 53, 63, 73, 83, 93, 103, 113, 123, 133, 143, 153, 163, 173, 183, 193, 203, 213, 223, 233, 243, 253, 263, 273, 283, 293, 303, 313, 323, and 333.

2. The antibody or antigen-binding portion thereof of claim 1, wherein the VH-CDR2 comprises an amino acid sequence selected from the sequences set forth in SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, and 332.

3. The antibody or antigen-binding portion thereof of claim 1 or 2, wherein the VH-CDR1 comprises an amino acid sequence selected from the sequences set forth in SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, and 331.

4. The antibody or antigen-binding portion thereof of any one of claims 1 to 3, wherein the VL-CDR3 comprises an amino acid sequence selected from the sequences set forth in SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, and 336.

5. The antibody or antigen-binding portion thereof of any one of claims 1 to 4, wherein the VL-CDR2 comprises an amino acid sequence selected from the sequences set forth in SEQ ID NOs: 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, and 335.

6. The antibody or antigen-binding portion thereof of any one of claims 1 to 5, wherein the VL-CDR1 comprises an amino acid sequence selected from the sequences set forth in SEQ ID NOs: 4, 14, 24, 34, 44, 54, 64, 74, 84, 94, 104, 114, 124, 134, 144, 154, 164, 174, 184, 194, 204, 214, 224, 234, 244, 254, 264, 274, 284, 294, 304, 314, 324, and 334.

7. The antibody or antigen-binding portion thereof of any one of claims 1 to 6, comprising:
(i) a VH-CDR1 comprising the amino acid sequence set forth in SEQ ID NO: 1,
a VH-CDR2 comprising the amino acid sequence set forth in SEQ ID NO: 2,
a VH-CDR3 comprising the amino acid sequence set forth in SEQ ID NO: 3,
a VL-CDR1 comprising the amino acid sequence set forth in SEQ ID NO: 4,
a VL-CDR2 comprising the amino acid sequence set forth in SEQ ID NO: 5, and a VL-CDR3 comprising the amino acid sequence set forth in SEQ ID NO: 6;
(ii) a VH-CDR1 comprising the amino acid sequence set forth in SEQ ID NO: 91,
a VH-CDR2 comprising the amino acid sequence set forth in SEQ ID NO: 92,
a VH-CDR3 comprising the amino acid sequence set forth in SEQ ID NO: 93,
a VL-CDR1 comprising the amino acid sequence set forth in SEQ ID NO: 94,
a VL-CDR2 comprising the amino acid sequence set forth in SEQ ID NO: 95, and
a VL-CDR3 comprising the amino acid sequence set forth in SEQ ID NO: 96;
(iii) a VH-CDR1 comprising the amino acid sequence set forth in SEQ ID NO: 111,
a VH-CDR2 comprising the amino acid sequence set forth in SEQ ID NO: 112,
a VH-CDR3 comprising the amino acid sequence set forth in SEQ ID NO: 113,
a VL-CDR1 comprising the amino acid sequence set forth in SEQ ID NO: 114,
a VL-CDR2 comprising the amino acid sequence set forth in SEQ ID NO: 115, and
a VL-CDR3 comprising the amino acid sequence set forth in SEQ ID NO: 116;
(iv) a VH-CDR1 comprising the amino acid sequence set forth in SEQ ID NO: 141,
a VH-CDR2 comprising the amino acid sequence set forth in SEQ ID NO: 142,
a VH-CDR3 comprising the amino acid sequence set forth in SEQ ID NO: 143,
a VL-CDR1 comprising the amino acid sequence set forth in SEQ ID NO: 144,
a VL-CDR2 comprising the amino acid sequence set forth in SEQ ID NO: 145, and
a VL-CDR3 comprising the amino acid sequence set forth in SEQ ID NO: 146;
(v) a VH-CDR1 comprising the amino acid sequence set forth in SEQ ID NO: 211,
a VH-CDR2 comprising the amino acid sequence set forth in SEQ ID NO: 212,
a VH-CDR3 comprising the amino acid sequence set forth in SEQ ID NO: 213,
a VL-CDR1 comprising the amino acid sequence set forth in SEQ ID NO: 214,
a VL-CDR2 comprising the amino acid sequence set forth in SEQ ID NO: 215, and
a VL-CDR3 comprising the amino acid sequence set forth in SEQ ID NO: 216;
(vi) a VH-CDR1 comprising the amino acid sequence set forth in SEQ ID NO: 221,
a VH-CDR2 comprising the amino acid sequence set forth in SEQ ID NO: 222,
a VH-CDR3 comprising the amino acid sequence set forth in SEQ ID NO: 223,
a VL-CDR1 comprising the amino acid sequence set forth in SEQ ID NO: 224,
a VL-CDR2 comprising the amino acid sequence set forth in SEQ ID NO: 225, and
a VL-CDR3 comprising the amino acid sequence set forth in SEQ ID NO: 226;
(vii) a VH-CDR1 comprising the amino acid sequence set forth in SEQ ID NO: 271,
a VH-CDR2 comprising the amino acid sequence set forth in SEQ ID NO: 272,
a VH-CDR3 comprising the amino acid sequence set forth in SEQ ID NO: 273,
a VL-CDR1 comprising the amino acid sequence set forth in SEQ ID NO: 274,
a VL-CDR2 comprising the amino acid sequence set forth in SEQ ID NO: 275, and
a VL-CDR3 comprising the amino acid sequence set forth in SEQ ID NO: 276;
(viii) a VH-CDR1 comprising the amino acid sequence set forth in SEQ ID NO: 281,
a VH-CDR2 comprising the amino acid sequence set forth in SEQ ID NO: 282,
a VH-CDR3 comprising the amino acid sequence set forth in SEQ ID NO: 283,
a VL-CDR1 comprising the amino acid sequence set forth in SEQ ID NO: 284,
a VL-CDR2 comprising the amino acid sequence set forth in SEQ ID NO: 285, and
a VL-CDR3 comprising the amino acid sequence set forth in SEQ ID NO: 286;
(ix) a VH-CDR1 comprising the amino acid sequence set forth in SEQ ID NO: 291,
a VH-CDR2 comprising the amino acid sequence set forth in SEQ ID NO: 292,
a VH-CDR3 comprising the amino acid sequence set forth in SEQ ID NO: 293,
a VL-CDR1 comprising the amino acid sequence set forth in SEQ ID NO: 294,
a VL-CDR2 comprising the amino acid sequence set forth in SEQ ID NO: 295, and
a VL-CDR3 comprising the amino acid sequence set forth in SEQ ID NO: 296;
(x) a VH-CDR1 comprising the amino acid sequence set forth in SEQ ID NO: 301,
a VH-CDR2 comprising the amino acid sequence set forth in SEQ ID NO: 302,
a VH-CDR3 comprising the amino acid sequence set forth in SEQ ID NO: 303,
a VL-CDR1 comprising the amino acid sequence set forth in SEQ ID NO: 304,
a VL-CDR2 comprising the amino acid sequence set forth in SEQ ID NO: 305, and
a VL-CDR3 comprising the amino acid sequence set forth in SEQ ID NO: 306; or
(xi) any combination of (i) to (x).

8. The antibody or antigen-binding portion thereof of any one of claims 1 to 7, wherein the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 707, 317, 327, and 337.

9. The antibody or antigen-binding portion thereof of any one of claims 1 to 8, wherein the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, and 338.

10. An antibody or an antigen-binding portion thereof that specifically binds CD161, comprising a variable heavy (VH) domain and a variable light (VL) domain, wherein the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 707, 317, 327, and 337; and wherein the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, and 338.

11. The antibody or antigen-binding portion thereof of any one of claims 1 to 10, wherein the VH comprises an amino acid sequence selected from SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 707, 317, 327, and 337.

12. The antibody or antigen-binding portion thereof of any one of claims 1 to 11, wherein the VL comprises an amino acid sequence selected from SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, and 338.

13. The antibody or antigen-binding portion thereof of any one of claims 1 to 12, wherein
(i) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 7; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 8;
(ii) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 97; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 98;
(iii) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 117; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 118;
(iv) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 147; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 148;
(v) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 217; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 218;
(vi) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 227; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 228;
(vii) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 277; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 278;
(viii) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 287; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 288;
(ix) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 297; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 298;
(x) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 307; and the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 308; or
(xi) any combination of (i) to (x).

14. The antibody or antigen-binding portion thereof of any one of claims 1 to 13, comprising:
(i) a VH comprising the amino acid sequence set forth in SEQ ID NO: 7, and a VL comprising the amino acid sequence set forth in SEQ ID NO: 8;
(ii) a VH comprising the amino acid sequence set forth in SEQ ID NO: 97, and a VL comprising the amino acid sequence set forth in SEQ ID NO: 98;
(iii) a VH comprising the amino acid sequence set forth in SEQ ID NO: 117, and a VL comprising the amino acid sequence set forth in SEQ ID NO: 118;
(iv) a VH comprising the amino acid sequence set forth in SEQ ID NO: 147, and a VL comprising the amino acid sequence set forth in SEQ ID NO: 148;
(v) a VH comprising the amino acid sequence set forth in SEQ ID NO: 217, and a VL comprising the amino acid sequence set forth in SEQ ID NO: 218;
(vi) a VH comprising the amino acid sequence set forth in SEQ ID NO: 227, and a VL comprising the amino acid sequence set forth in SEQ ID NO: 228;
(vii) a VH comprising the amino acid sequence set forth in SEQ ID NO: 277, and a VL comprising the amino acid sequence set forth in SEQ ID NO: 278;
(viii) a VH comprising the amino acid sequence set forth in SEQ ID NO: 287, and a VL comprising the amino acid sequence set forth in SEQ ID NO: 288;
(ix) a VH comprising the amino acid sequence set forth in SEQ ID NO: 297, and a VL comprising the amino acid sequence set forth in SEQ ID NO: 298;
(x) a VH comprising the amino acid sequence set forth in SEQ ID NO: 307, and a VL comprising the amino acid sequence set forth in SEQ ID NO: 308;
(xi) any combination of (i) to (x).

15. An antibody or antigen-binding portion thereof that binds the same epitope as the antibody or antigen-binding portion thereof of any one of claims 1 to 14.

16. An antibody or antigen-binding portion thereof that cross-competes for binding CD161 with the antibody or antigen-binding portion thereof of any one of claims 1 to 14.

17. The antibody or antigen-binding portion thereof of any one of claims 1 to 16, which binds CD161 with a K_{D} of less than about 1000 nM, less than about 500 nM, less than about 100 nM, less than about 50 nM, or less than about 10 nM.

18. The antibody or antigen-binding portion thereof of any one of claims 1 to 17, which binds CD161 with a K_{D} of less than about 50 nM.

19. The antibody or antigen-binding portion thereof of any one of claims 1 to 18, which binds CD161 with a K_{D} of less than about 10 nM.

20. The antibody or antigen-binding portion thereof of any one of claims 1 to 19, which inhibits the interaction between CD161 and C-type lectin domain family 2 member D (CLEC2D).

21. The antibody or antigen-binding portion thereof of any one of claims 1 to 20, which is capable of inducing or enhancing production of one or more cytokine by an immune cell.

22. The antibody or antigen-binding portion thereof of claim 21, wherein the one or more cytokine comprises IL2, TNFa, IFNg, or any combination thereof.

23. The antibody or antigen-binding portion thereof of any one of claims 1 to 22, wherein the antigen-binding portion of the antibody comprises a VHH, a vNAR, a microbody, a nanobody, an scFv, or any combination thereof.

24. A multi-specific antibody comprising the antibody or antigen-binding portion thereof of any one of claims 1 to 23.

25. A bispecific antibody comprising the antibody or antigen-binding portion thereof of any one of claims 1 to 23.

26. A nucleic acid molecule or a set of nucleic acid molecules encoding the antibody or antigen-binding portion thereof of any one of claims 1 to 23, the multi-specific antibody of claim 24, or the bispecific antibody of claim 25.

27. A vector or a set of vectors comprising the nucleic acid molecule or the set of nucleic acid molecules of claim 26.

28. The vector or the set of vectors of claim 36, which is a viral vector.

29. A host cell comprising the nucleic acid molecule or the set of nucleic acid molecules of claim 26 or the vector or the set of vectors of claim 27 or 28.

30. A pharmaceutical composition comprising the antibody or antigen-binding portion thereof of any one of claims 1 to 23, the multi-specific antibody of claim 24, the bispecific antibody of claim 25, the nucleic acid molecule or the set of nucleic acid molecules of claim 26, the vector or the set of vectors of claim 27 or 28, or the host cell of claim 29 and a pharmaceutically acceptable carrier.

31. A method of treating a disease or disorder in a subject in need thereof, comprising administering to the subject the antibody or antigen-binding portion thereof of any one of claims 1 to 23, the multi-specific antibody of claim 24, the bispecific antibody of claim 25, the nucleic acid molecule or the set of nucleic acid molecules of claim 26, the vector or the set of vectors of claim 27 or 28, the host cell of claim 29, or the pharmaceutical composition of claim 30.

32. The method of claim 31, wherein the disease or disorder comprises a cancer.

33. A method of inducing an immune response in a subject in need thereof, comprising administering to the subject the antibody or antigen-binding portion thereof of any one of claims 1 to 23, the multi-specific antibody of claim 24, the bispecific antibody of claim 25, the nucleic acid molecule or the set of nucleic acid molecules of claim 26, the vector or the set of vectors of claim 27 or 28, the host cell of claim 29, or the pharmaceutical composition of claim 30.

34. The method of claim 33, wherein the subject is afflicted with a cancer.

35. A method of treating a cancer in a subject in need thereof, comprising administering to the subject the antibody or antigen-binding portion thereof of any one of claims 1 to 23, the multi-specific antibody of claim 24, the bispecific antibody of claim 25, the nucleic acid molecule or the set of nucleic acid molecules of claim 26, the vector or the set of vectors of claim 27 or 28, the host cell of claim 29, or the pharmaceutical composition of claim 30.

36. The method of any one of claims 32, 34, and 35, wherein the cancer is selected from acoustic neuroma, acute lymphocytic leukemia, acute myelocytic leukemia, adenocarcinoma, and cancer of the urinary system, and carcinomas, angiosarcoma, astrocytoma, basal cell carcinoma, bile duct carcinoma, biliary tract cancer, bladder cancer, bone cancer, brain cancer, brain stem glioma, breast cancer, bronchogenic carcinoma, Burkitt's lymphoma and marginal zone B cell lymphoma, cancer of the adrenal gland, cancer of the anal region, cancer of the digestive system, cancer of the endocrine system, cancer of the esophagus, cancer of the parathyroid gland, cancer of the penis, cancer of the respiratory system, cancer of the small intestine, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, central nervous system (CNS) cancer, cervical cancer, chondrosarcoma, chordoma, choriocarcinoma, chronic leukemia, chronic lymphocytic leukemia, chronic myelocytic (granulocytic) leukemia, colon carcinoma, colon sarcoma, colorectal cancer, connective tissue cancer, craniopharyngioma, cystadenocarcinoma, embryonal carcinoma, endometrial cancer, endotheliosarcoma, environmentally-induced cancers including those induced by asbestos, ependymoma, epidermoid cancer, epithelial carcinoma, esophageal cancer, esophageal carcinoma, Ewing's tumor, eye cancer, fibrosarcoma, gastric cancer, gastrointestinal cancer, germ cell tumor, glioblastoma (e.g. glioblastoma multiforme), glioma, head and neck cancer, heavy chain disease, hemangioblastoma, hepatoma, Hodgkin's disease, intraepithelial neoplasm, Kaposi's sarcoma, kidney cancer (e.g.renal cell carcinoma (RCC)), larynx cancer, leiomyosarcoma, leukemia, liposarcoma, liver cancer, lung cancer (small cell, large cell), lung carcinoma, lymphangioendotheliosarcoma, lymphangiosarcoma, mantle cell lymphoma, medullary carcinoma, medulloblastoma, melanoma, menangioma, mesothelioma, multiple myeloma, myeloblasts promyelocyte myelomonocytic monocytic erythroleukemia, myxosarcoma, nasopharyngeal carcinoma, neoplasm of the central nervous system (CNS), neuroblastoma, non-Hodgkin's disease, non-small cell lung cancer (NSCLC), non-small cell lung carcinoma, oligodendroglioma, oral cavity cancer (for example lip, tongue, mouth and pharynx), osteogenic sarcoma, osteosarcoma, ovarian cancer, pancreatic cancer, papillary adenocarcinomas, papillary carcinoma, pediatric sarcoma, pinealoma, pituitary adenoma, Polycythemia vera Lymphoma, primary CNS lymphoma, prostate cancer (e.g. hormone refractory prostate adenocarcinoma), rectal cancer, renal cancer (e.g. clear cell carcinoma), retinoblastoma, rhabdomyosarcoma, sarcoma, sarcoma of soft tissue, sebaceous gland carcinoma, seminoma, sinonasal natural killer, skin cancer, small-cell lung cancer (SCLC), solid tumors of childhood, spinal axis tumor, squamous cell cancer, squamous cell carcinoma, stomach cancer, sweat gland carcinoma, synovioma, testicular cancer, thyroid cancer, tumor angiogenesis, uterine cancer, virus-related cancers or cancers of viral origin (e.g. human papilloma virus (HPV-related or- originating tumors)), Waldenstrom's macroglobulinemia, and Wilm's tumor; and any combinations of said cancers.

37. A method of treating an infectious disease in a subject in need thereof, comprising administering to the subject the antibody or antigen-binding portion thereof of any one of claims 1 to 23, the multi-specific antibody of claim 24, the bispecific antibody of claim 25, the nucleic acid molecule or the set of nucleic acid molecules of claim 26, the vector or the set of vectors of claim 27 or 28, the host cell of claim 29, or the pharmaceutical composition of claim 30.

38. The method of claim 37, wherein the infectious disease comprises:
(i) infection by Influenza, Herpes, Giardia, Malaria, Leishmania, or any combination thereof;
(ii) infection by human immunodeficiency virus (HIV), Hepatitis virus herpes virus, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus, or arboviral encephalitis virus, or any combination thereof;
(iii) infection by chlamydia, rickettsial bacteria, mycobacteria, staphylococci, streptococci, pneumonococci, meningococci, conococci, klebsiella, proteus, serratia, pseudomonas, legionella, diphtheria, salmonella, bacilli, cholera, tetanus, botulism, anthrax, plague, leptospirosis, and Lyme's disease bacteria, or any combination thereof;
(iv) infection by Candida, Cryptococcus neoformans, Aspergillus, genus Mucorales, Sporothrix schenkii, Blastomyces dermatitidis, Paracoccidioides brasiliensis, Coccidioides immitis, or Histoplasma capsulatum, or any combination thereof;
(v) infection by Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, or Nippostrongylus brasiliensis, or any combination thereof, or
(vi) any combination of (i) to (v).

39. A method of treating an autoimmune disease in subject in need thereof, comprising administering to the subject the antibody or antigen-binding portion thereof of any one of claims 1 to 23, the multi-specific antibody of claim 24, the bispecific antibody of claim 25, the nucleic acid molecule or the set of nucleic acid molecules of claim 26, the vector or the set of vectors of claim 27 or 28, the host cell of claim 29, or the pharmaceutical composition of claim 30.

40. A method of activating an immune cell, comprising contacting the immune cell with the antibody or antigen-binding portion thereof of any one of claims 1 to 23, the multi-specific antibody of claim 24, the bispecific antibody of claim 25, the nucleic acid molecule or the set of nucleic acid molecules of claim 26, the vector or the set of vectors of claim 27 or 28, the host cell of claim 29, or the pharmaceutical composition of claim 30.


SEQUENCE LISTING (341 sequences)

SEQ ID NO: 1 - protein, 5 aa, synthetic construct
  source 1..5: note = VH-CDR1
SNYWT

SEQ ID NO: 2 - protein, 16 aa, synthetic construct
  source 1..16: note = VH-CDR2
YIYYIGTTNYNPSLKS

SEQ ID NO: 3 - protein, 15 aa, synthetic construct
  source 1..15: note = VH-CDR3
ARDGGYSGTYWGLDP

SEQ ID NO: 4 - protein, 12 aa, synthetic construct
  source 1..12: note = VL-CDR1
RASQSVSSSHLA

SEQ ID NO: 5 - protein, 7 aa, synthetic construct
  source 1..7: note = VL-CDR2
GASSRAT

SEQ ID NO: 6 - protein, 9 aa, synthetic construct
  source 1..9: note = VL-CDR3
QQYGSSPIT

SEQ ID NO: 7 - protein, 121 aa, synthetic construct
  source 1..121: note = VH
QVQLQESGPGLVKPSETLSLTCTVSGGSISSNYWTWIRQPPGKGLEWIGYIYYIGTTNYNPSLKSRVTISLDTSKNHFSLRLSSVTAADTAVYYCARDGGYSGTYWGLDPWGQGTLVTVSS

SEQ ID NO: 8 - protein, 108 aa, synthetic construct
  source 1..108: note = VL
EIVLTQSPGTLSLSPGERATLSCRASQSVSSSHLAWYQQKPGQAPRLLIYGASSRATGIPDRFSGSGSGTDFTLTISRLEPEDFAVYNCQQYGSSPITFGQGTRLEIK

SEQ ID NO: 9-10 - skipped (2 entries)

SEQ ID NO: 11 - protein, 5 aa, synthetic construct
  source 1..5: note = VH-CDR1
SYYWS

SEQ ID NO: 12 - protein, 16 aa, synthetic construct
  source 1..16: note = VH-CDR2
YVYNSESTYYNPSLKS

SEQ ID NO: 13 - protein, 13 aa, synthetic construct
  source 1..13: note = VH-CDR3
DGGIVGLTAAFDI

SEQ ID NO: 14 - protein, 12 aa, synthetic construct
  source 1..12: note = VL-CDR1
RASQSVSSNYLA

SEQ ID NO: 15 - protein, 7 aa, synthetic construct
  source 1..7: note = VL-CDR2
GASSRAT

SEQ ID NO: 16 - protein, 9 aa, synthetic construct
  source 1..9: note = VL-CDR3
QQYVNSPIT

SEQ ID NO: 17 - protein, 121 aa, synthetic construct
  source 1..121: note = VH
QVHLQESGPGLVKPSETLSLTCTVSGGSIISYYWSWIRQPPGKGLEWIGYVYNSESTYYNPSLKSRVTISEDTSKSQFSLKLSSVTAADTAVYYCARDGGIVGLTAAFDIWGQGTMVTVSS

SEQ ID NO: 18 - protein, 108 aa, synthetic construct
  source 1..108: note = VL
EIVLTQSPGTLSLSPGERATLSCRASQSVSSNYLAWYQQKPGQAPRLLIYGASSRATGIPDRFSGNGSGTDFTLTISRLEPEDFAVYYCQQYVNSPITFGPGTKVDIK

SEQ ID NO: 19-20 - skipped (2 entries)

SEQ ID NO: 21 - protein, 5 aa, synthetic construct
  source 1..5: note = VH-CDR1
SYYWS

SEQ ID NO: 22 - protein, 16 aa, synthetic construct
  source 1..16: note = VH-CDR2
YIYYIGSTNYNPSLKS

SEQ ID NO: 23 - protein, 8 aa, synthetic construct
  source 1..8: note = VH-CDR3
DRMGALDY

SEQ ID NO: 24 - protein, 16 aa, synthetic construct
  source 1..16: note = VL-CDR1
RSSQSLVHSDGNTYLS

SEQ ID NO: 25 - protein, 7 aa, synthetic construct
  source 1..7: note = VL-CDR2
KISNRFS

SEQ ID NO: 26 - protein, 8 aa, synthetic construct
  source 1..8: note = VL-CDR3
MQAAQFPT

SEQ ID NO: 27 - protein, 116 aa, synthetic construct
  source 1..116: note = VH
QVQLQESGPGLVKPSETLSLTCTVSGGSISSYYWSWIRQPPGKGLEWIGYIYYIGSTNYNPSLKSRVTISGDTSKNQFSLKLTSVTAADTAVYYCARDRMGALDYWGQGTLVTVSS

SEQ ID NO: 28 - protein, 111 aa, synthetic construct
  source 1..111: note = VL
DIVMTQTPLSSPVTLGQPASISCRSSQSLVHSDGNTYLSWLQQRPGQPPRLLIYKISNRFSGVPDRFSGSGAGTDFTLIISRVEAEDVGVYYCMQAAQFPTFGGGTKVEIK

SEQ ID NO: 29-30 - skipped (2 entries)

SEQ ID NO: 31 - protein, 5 aa, synthetic construct
  source 1..5: note = VH-CDR1
SYYWS

SEQ ID NO: 32 - protein, 16 aa, synthetic construct
  source 1..16: note = VH-CDR2
RIYTIGTTNYNPSLKS

SEQ ID NO: 33 - protein, 9 aa, synthetic construct
  source 1..9: note = VH-CDR3
DPLGSFFDY

SEQ ID NO: 34 - protein, 14 aa, synthetic construct
  source 1..14: note = VL-CDR1
TGTSSDVGGYNYVS

SEQ ID NO: 35 - protein, 7 aa, synthetic construct
  source 1..7: note = VL-CDR2
EVSKRPS

SEQ ID NO: 36 - protein, 10 aa, synthetic construct
  source 1..10: note = VL-CDR3
SSYAGSNNLV

SEQ ID NO: 37 - protein, 117 aa, synthetic construct
  source 1..117: note = VH
QVQLQESGPGLVKPSETLSLTCTVSGGSISSYYWSWIRQPAGKGLEWIGRIYTIGTTNYNPSLKSRVTMSVDTSKNQFSLKLSSVTAADTAVYYCARDPLGSFFDYWGQGSLVTVSS

SEQ ID NO: 38 - protein, 110 aa, synthetic construct
  source 1..110: note = VL
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVFGGGTKLTVL

SEQ ID NO: 39-40 - skipped (2 entries)

SEQ ID NO: 41 - protein, 5 aa, synthetic construct
  source 1..5: note = VH-CDR1
SYYWS

SEQ ID NO: 42 - protein, 16 aa, synthetic construct
  source 1..16: note = VH-CDR2
RTYTSGSTNYNPSLKS

SEQ ID NO: 43 - protein, 9 aa, synthetic construct
  source 1..9: note = VH-CDR3
DPLGTFFDY

SEQ ID NO: 44 - protein, 14 aa, synthetic construct
  source 1..14: note = VL-CDR1
TGTSSDVGGYNYVS

SEQ ID NO: 45 - protein, 7 aa, synthetic construct
  source 1..7: note = VL-CDR2
EVSKRPS

SEQ ID NO: 46 - protein, 10 aa, synthetic construct
  source 1..10: note = VL-CDR3
SSYAGINNVV

SEQ ID NO: 47 - protein, 117 aa, synthetic construct
  source 1..117: note = VH
QVQLQESGPGLVKPSETLSLTCTVSGDSINSYYWSWIRQPAGKGLEWIGRTYTSGSTNYNPSLKSRVTMSVDTSKKQISLKLSSVTAADTAVYYCARDPLGTFFDYWGQGILVTVSS

SEQ ID NO: 48 - protein, 110 aa, synthetic construct
  source 1..110: note = VL
QSALTQPPSASGSPGQSVTISCTGTSSDVGGYNYVSWYQQHPGKAPKFMIYEVSKRPSGVPDRFSGSKSGNTASLTVSGLQAEDEADYYCSSYAGINNVVFGGGTKLTVL

SEQ ID NO: 49-50 - skipped (2 entries)

SEQ ID NO: 51 - protein, 5 aa, synthetic construct
  source 1..5: note = VH-CDR1
SYYWS

SEQ ID NO: 52 - protein, 16 aa, synthetic construct
  source 1..16: note = VH-CDR2
YIYYIGTTNYNPSLKS

SEQ ID NO: 53 - protein, 8 aa, synthetic construct
  source 1..8: note = VH-CDR3
DRMGALDY

SEQ ID NO: 54 - protein, 16 aa, synthetic construct
  source 1..16: note = VL-CDR1
RSSQSLVHSDGNTYLS

SEQ ID NO: 55 - protein, 7 aa, synthetic construct
  source 1..7: note = VL-CDR2
EISNRFS

SEQ ID NO: 56 - protein, 8 aa, synthetic construct
  source 1..8: note = VL-CDR3
MQAAQFPT

SEQ ID NO: 57 - protein, 116 aa, synthetic construct
  source 1..116: note = VH
QVQLQESGPGLVKPSETLSLTCTVSGGSISSYYWSWIRQPPGKGLEWIGYIYYIGTTNYNPSLKSRVTISVDTSKNQFSLKLSSVTAADTAVYYCARDRMGALDYWGQGTLVTVSS

SEQ ID NO: 58 - protein, 111 aa, synthetic construct
  source 1..111: note = VL
DIVMTQTPLSSPVTLGQPASISCRSSQSLVHSDGNTYLSWLQQRPGQPPRLLIYEISNRFSGVPDRFSGSGAGTDFTLKISRVEAEDVGVYYCMQAAQFPTFGGGTKVEIK

SEQ ID NO: 59-60 - skipped (2 entries)

SEQ ID NO: 61 - protein, 5 aa, synthetic construct
  source 1..5: note = VH-CDR1
NYYWS

SEQ ID NO: 62 - protein, 16 aa, synthetic construct
  source 1..16: note = VH-CDR2
RIYTSGSNNYNPSLKS

SEQ ID NO: 63 - protein, 9 aa, synthetic construct
  source 1..9: note = VH-CDR3
DPLGTFFDY

SEQ ID NO: 64 - protein, 14 aa, synthetic construct
  source 1..14: note = VL-CDR1
TGTSSDVGGYNYVS

SEQ ID NO: 65 - protein, 7 aa, synthetic construct
  source 1..7: note = VL-CDR2
EVSKRPS

SEQ ID NO: 66 - protein, 10 aa, synthetic construct
  source 1..10: note = VL-CDR3
SSYADTNNVV

SEQ ID NO: 67 - protein, 117 aa, synthetic construct
  source 1..117: note = VH
QVQLQESGPGLVKPSETLSLTCTVSGGSISNYYWSWIRQPAGKGLEWIGRIYTSGSNNYNPSLKSRVTMSVDTSKKQFSLKLSSVTAADTAVYYCARDPLGTFFDYWGQGTLVTVSS

SEQ ID NO: 68 - protein, 110 aa, synthetic construct
  source 1..110: note = VL
QSALTQPPSASGSPGQSVTISCTGTSSDVGGYNYVSWYQQHPGKAPKLMIYEVSKRPSGVPDRFSGSKSGNTASLTVSGLQAEDEADYYCSSYADTNNVVFGGGTKLTVL

SEQ ID NO: 69-70 - skipped (2 entries)

SEQ ID NO: 71 - protein, 5 aa, synthetic construct
  source 1..5: note = VH-CDR1
SYFWS

SEQ ID NO: 72 - protein, 16 aa, synthetic construct
  source 1..16: note = VH-CDR2
YIYYNGHTNYNPSLKS

SEQ ID NO: 73 - protein, 10 aa, synthetic construct
  source 1..10: note = VH-CDR3
DSGPWVPLDY

SEQ ID NO: 74 - protein, 12 aa, synthetic construct
  source 1..12: note = VL-CDR1
RASQSVISSYLA

SEQ ID NO: 75 - protein, 7 aa, synthetic construct
  source 1..7: note = VL-CDR2
GASSRAT

SEQ ID NO: 76 - protein, 9 aa, synthetic construct
  source 1..9: note = VL-CDR3
QQYGSSPPT

SEQ ID NO: 77 - protein, 118 aa, synthetic construct
  source 1..118: note = VH
QVQLQESGPGLVKPSETLSLTCTVSGGSISSYFWSWIRQPPGKGLEWIGYIYYNGHTNYNPSLKSRVTISVDTSKNQFSLKLSSVTAADTAVYYCARDSGPWVPLDYWGQGTLVTVSS

SEQ ID NO: 78 - protein, 108 aa, synthetic construct
  source 1..108: note = VL
EIVLTQSPGTLSLSPGERATLSCRASQSVISSYLAWYQQKPGQAPRLLIYGASSRATGIPDRFSGSGSGTDFTLTISRLEPEDFAVYFCQQYGSSPPTFGQGTKVEIK

SEQ ID NO: 79-80 - skipped (2 entries)

SEQ ID NO: 81 - protein, 5 aa, synthetic construct
  source 1..5: note = VH-CDR1
FYYWS

SEQ ID NO: 82 - protein, 16 aa, synthetic construct
  source 1..16: note = VH-CDR2
YIYHSGSTNYNPSLKS

SEQ ID NO: 83 - protein, 13 aa, synthetic construct
  source 1..13: note = VH-CDR3
DGGIVGVTEAIDI

SEQ ID NO: 84 - protein, 12 aa, synthetic construct
  source 1..12: note = VL-CDR1
RASQVISSSYLA

SEQ ID NO: 85 - protein, 7 aa, synthetic construct
  source 1..7: note = VL-CDR2
GASSRAT

SEQ ID NO: 86 - protein, 9 aa, synthetic construct
  source 1..9: note = VL-CDR3
QQYGSSPLT

SEQ ID NO: 87 - protein, 121 aa, synthetic construct
  source 1..121: note = VH
QVQLQESGPGLVKPSETLSLTCTVSGGSISFYYWSWIRQPPGKGLEWIGYIYHSGSTNYNPSLKSRVTISVDTSKNQFSLKLSSVTAADTAVYYCARDGGIVGVTEAIDIWGQGTVVTVSS

SEQ ID NO: 88 - protein, 108 aa, synthetic construct
  source 1..108: note = VL
EVVLTQTPGTLSLSPGERATLSCRASQVISSSYLAWYQQKPGQAPRLLIFGASSRATGIPDRFSGSGSGTDFTLTISRLEPADFAVYYCQQYGSSPLTFGGGTKVEIK

SEQ ID NO: 89-90 - skipped (2 entries)

SEQ ID NO: 91 - protein, 5 aa, synthetic construct
  source 1..5: note = VH-CDR1
SYYWS

SEQ ID NO: 92 - protein, 16 aa, synthetic construct
  source 1..16: note = VH-CDR2
YIYYSGSANYNPSLKS

SEQ ID NO: 93 - protein, 13 aa, synthetic construct
  source 1..13: note = VH-CDR3
ASDRGPWGGGFDY

SEQ ID NO: 94 - protein, 12 aa, synthetic construct
  source 1..12: note = VL-CDR1
RASQSVSSSYLA

SEQ ID NO: 95 - protein, 7 aa, synthetic construct
  source 1..7: note = VL-CDR2
GASSRAT

SEQ ID NO: 96 - protein, 9 aa, synthetic construct
  source 1..9: note = VL-CDR3
QQYGNSPLT

SEQ ID NO: 97 - protein, 119 aa, synthetic construct
  source 1..119: note = VH
QVQLLESGPGLVKPSETLSLTCTVSNGSIRSYYWSWIRQPPGKGLEWIGYIYYSGSANYNPSLKSRVTISVDTSETQFSLKLSSVTAADTAVYYCASDRGPWGGGFDYWGQGTLVTVSS

SEQ ID NO: 98 - protein, 108 aa, synthetic construct
  source 1..108: note = VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGNSPLTFGGGTKVEIK

SEQ ID NO: 99-100 - skipped (2 entries)

SEQ ID NO: 101 - protein, 5 aa, synthetic construct
  source 1..5: note = VH-CDR1
SYYWS

SEQ ID NO: 102 - protein, 16 aa, synthetic construct
  source 1..16: note = VH-CDR2
YIYYNGNTNYNPSLKS

SEQ ID NO: 103 - protein, 10 aa, synthetic construct
  source 1..10: note = VH-CDR3
DSGPWVPLDY

SEQ ID NO: 104 - protein, 12 aa, synthetic construct
  source 1..12: note = VL-CDR1
RASQSVSSSYLA

SEQ ID NO: 105 - protein, 7 aa, synthetic construct
  source 1..7: note = VL-CDR2
GASSRAT

SEQ ID NO: 106 - protein, 9 aa, synthetic construct
  source 1..9: note = VL-CDR3
QQYGSSPPT

SEQ ID NO: 107 - protein, 118 aa, synthetic construct
  source 1..118: note = VH
QVQLQESGPGLVQPSETLSLTCTVSGSSISSYYWSWIRQPPGKGLEWIGYIYYNGNTNYNPSLKSRVTLSVDTSKNQFSLKVRSVTAADTAVYYCARDSGPWVPLDYWGQGTLVTVSS

SEQ ID NO: 108 - protein, 108 aa, synthetic construct
  source 1..108: note = VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPTFGQGTKVEIK

SEQ ID NO: 109-110 - skipped (2 entries)

SEQ ID NO: 111 - protein, 5 aa, synthetic construct
  source 1..5: note = VH-CDR1
TYDMH

SEQ ID NO: 112 - protein, 16 aa, synthetic construct
  source 1..16: note = VH-CDR2
TIGTAGDTKYSGSVKG

SEQ ID NO: 113 - protein, 11 aa, synthetic construct
  source 1..11: note = VH-CDR3
TRAPVGAISDY

SEQ ID NO: 114 - protein, 13 aa, synthetic construct
  source 1..13: note = VL-CDR1
TRSSGSIASNYVQ

SEQ ID NO: 115 - protein, 7 aa, synthetic construct
  source 1..7: note = VL-CDR2
EDNQRPS

SEQ ID NO: 116 - protein, 10 aa, synthetic construct
  source 1..10: note = VL-CDR3
SYDSINHVVF

SEQ ID NO: 117 - protein, 117 aa, synthetic construct
  source 1..117: note = VH
EVQLVESGGGLVQPGGSLRLSCAASGFTFSTYDMHWVRQVTGKGLEWVSTIGTAGDTKYSGSVKGRFTISRENAKNSLYLQMHSLRVGDTAVYYCTRAPVGAISDYWGQGTLVTVSS

SEQ ID NO: 118 - protein, 111 aa, synthetic construct
  source 1..111: note = VL
NFMLTQPHSVSESPGKTVTISCTRSSGSIASNYVQWYQQRPGNSPTIVIYEDNQRPSGVPDRFSGSIDSSSNSASLTISGLKTEDEADYYCQSYDSINHVVFGGGTKLTVL

SEQ ID NO: 119-120 - skipped (2 entries)

SEQ ID NO: 121 - protein, 5 aa, synthetic construct
  source 1..5: note = VH-CDR1
SGGYY

SEQ ID NO: 122 - protein, 17 aa, synthetic construct
  source 1..17: note = VH-CDR2
IGYIYNSGSTYYNPSLK

SEQ ID NO: 123 - protein, 13 aa, synthetic construct
  source 1..13: note = VH-CDR3
DNGIVGATGGMDV

SEQ ID NO: 124 - protein, 12 aa, synthetic construct
  source 1..12: note = VL-CDR1
RASQSVSSSYLA

SEQ ID NO: 125 - protein, 7 aa, synthetic construct
  source 1..7: note = VL-CDR2
GASSRAT

SEQ ID NO: 126 - protein, 9 aa, synthetic construct
  source 1..9: note = VL-CDR3
QQYGSSPLT

SEQ ID NO: 127 - protein, 123 aa, synthetic construct
  source 1..123: note = VH
QVQLQESGPGLVKPSQTLSLTCTVSGGSISSGGYYWSWIRQHPGKGLEWIGYIYNSGSTYYNPSLKSRVTISVDTSKNQFSLKLSSVTAADTAVYFCAGDNGIVGATGGMDVWGQGTTVTVSS

SEQ ID NO: 128 - protein, 108 aa, synthetic construct
  source 1..108: note = VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIK

SEQ ID NO: 129-130 - skipped (2 entries)

SEQ ID NO: 131 - protein, 5 aa, synthetic construct
  source 1..5: note = VH-CDR1
SYYWS

SEQ ID NO: 132 - protein, 16 aa, synthetic construct
  source 1..16: note = VH-CDR2
YIYYIGSTNYNPSLKS

SEQ ID NO: 133 - protein, 8 aa, synthetic construct
  source 1..8: note = VH-CDR3
DRMGALDY

SEQ ID NO: 134 - protein, 16 aa, synthetic construct
  source 1..16: note = VL-CDR1
RSSQSLVHSDGNTYLT

SEQ ID NO: 135 - protein, 7 aa, synthetic construct
  source 1..7: note = VL-CDR2
EISNRFS

SEQ ID NO: 136 - protein, 8 aa, synthetic construct
  source 1..8: note = VL-CDR3
MQAAQFPT

SEQ ID NO: 137 - protein, 116 aa, synthetic construct
  source 1..116: note = VH
QVQLQESGPGLVKPSETLSLTCTVSGGSINSYYWSWIRQPPGKGLEWIGYIYYIGSTNYNPSLKSRVTISMDTSKNQFSLKLSSVTAADTAVYYCARDRMGALDYWGQGTLVTVSS

SEQ ID NO: 138 - protein, 111 aa, synthetic construct
  source 1..111: note = VL
DIVMTQTPLSSPVTLGQPASISCRSSQSLVHSDGNTYLTWLQQRPGQPPRLLIYEISNRFSGVPDRFSGSGAGTDFTLKISRVEVEDVGVYYCMQAAQFPTFGQGTKLEIK

SEQ ID NO: 139-140 - skipped (2 entries)

SEQ ID NO: 141 - protein, 5 aa, synthetic construct
  source 1..5: note = VH-CDR1
YYYWS

SEQ ID NO: 142 - protein, 16 aa, synthetic construct
  source 1..16: note = VH-CDR2
YIYHSGSTNYNPSLKS

SEQ ID NO: 143 - protein, 15 aa, synthetic construct
  source 1..15: note = VH-CDR3
ARDGGIVGATGGFDY

SEQ ID NO: 144 - protein, 12 aa, synthetic construct
  source 1..12: note = VL-CDR1
RASQSVSSSYLA

SEQ ID NO: 145 - protein, 7 aa, synthetic construct
  source 1..7: note = VL-CDR2
GASSRAT

SEQ ID NO: 146 - protein, 9 aa, synthetic construct
  source 1..9: note = VL-CDR3
QQYGSSPIT

SEQ ID NO: 147 - protein, 121 aa, synthetic construct
  source 1..121: note = VH
QVQLQESGPGLVKPSETLSLTCTVSGGSISYYYWSWIRQPPGKGLEWIGYIYHSGSTNYNPSLKSRVTISIDTSENQFSLKLTSVTAADTAVYYCARDGGIVGATGGFDYWGQGTLVTVSS

SEQ ID NO: 148 - protein, 108 aa, synthetic construct
  source 1..108: note = VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTRLEIK

SEQ ID NO: 149-150 - skipped (2 entries)

SEQ ID NO: 151 - protein, 5 aa, synthetic construct
  source 1..5: note = VH-CDR1
SYYWS

SEQ ID NO: 152 - protein, 16 aa, synthetic construct
  source 1..16: note = VH-CDR2
RIYTSGSTNKNPSLKS

SEQ ID NO: 153 - protein, 9 aa, synthetic construct
  source 1..9: note = VH-CDR3
DPLGSFFDY

SEQ ID NO: 154 - protein, 14 aa, synthetic construct
  source 1..14: note = VL-CDR1
TGTSSDVGGYNYVS

SEQ ID NO: 155 - protein, 7 aa, synthetic construct
  source 1..7: note = VL-CDR2
EVSERPS

SEQ ID NO: 156 - protein, 10 aa, synthetic construct
  source 1..10: note = VL-CDR3
SSYAGSNNVV

SEQ ID NO: 157 - protein, 117 aa, synthetic construct
  source 1..117: note = VH
QVQLQESGPGLVKPSETLSLTCTVSGGSISSYYWSWIRQPAGKGLEWIGRIYTSGSTNKNPSLKSRITMSEDRSKNQFSLKLSSVTAADTAVYYCARDPLGSFFDYWGQGTLVIVSS

SEQ ID NO: 158 - protein, 110 aa, synthetic construct
  source 1..110: note = VL
QSALTQPPSASGSPGQSVTISCTGTSSDVGGYNYVSWYQQHPGKAPKFMIYEVSERPSGVPDRFSGSKSGNTASLTVSGLQAEDEADYYCSSYAGSNNVVFGGGTKLTVL

SEQ ID NO: 159-160 - skipped (2 entries)

SEQ ID NO: 161 - protein, 5 aa, synthetic construct
  source 1..5: note = VH-CDR1
SYYWS

SEQ ID NO: 162 - protein, 16 aa, synthetic construct
  source 1..16: note = VH-CDR2
RIYTIGTTNYNPSLKS

SEQ ID NO: 163 - protein, 9 aa, synthetic construct
  source 1..9: note = VH-CDR3
DPLGSFFDY

SEQ ID NO: 164 - protein, 14 aa, synthetic construct
  source 1..14: note = VL-CDR1
TGTSSDVGGYNYVS

SEQ ID NO: 165 - protein, 7 aa, synthetic construct
  source 1..7: note = VL-CDR2
EVSKRPS

SEQ ID NO: 166 - protein, 10 aa, synthetic construct
  source 1..10: note = VL-CDR3
SSYAGINNLV

SEQ ID NO: 167 - protein, 117 aa, synthetic construct
  source 1..117: note = VH
QVQLQESGPGLVKPSETLSLTCTVSGGSISSYYWSWIRQPAGKGLEWIGRIYTIGTTNYNPSLKSRVTMSVDTSKNQFSLKLSSVTAADAAVYYCARDPLGSFFDYWGQGSLVTVSS

SEQ ID NO: 168 - protein, 110 aa, synthetic construct
  source 1..110: note = VL
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGINNLVFGGGTKLTVL

SEQ ID NO: 169-170 - skipped (2 entries)

SEQ ID NO: 171 - protein, 5 aa, synthetic construct
  source 1..5: note = VH-CDR1
SYYWS

SEQ ID NO: 172 - protein, 16 aa, synthetic construct
  source 1..16: note = VH-CDR2
YIYYIGSTNYNPSLKS

SEQ ID NO: 173 - protein, 8 aa, synthetic construct
  source 1..8: note = VH-CDR3
DMMGALDY

SEQ ID NO: 174 - protein, 16 aa, synthetic construct
  source 1..16: note = VL-CDR1
RSSESLVHSDGNTYLS

SEQ ID NO: 175 - protein, 7 aa, synthetic construct
  source 1..7: note = VL-CDR2
KISNRFS

SEQ ID NO: 176 - protein, 8 aa, synthetic construct
  source 1..8: note = VL-CDR3
MQAAQFPT

SEQ ID NO: 177 - protein, 116 aa, synthetic construct
  source 1..116: note = VH
QVQLQESGPGLVKPSETLSLTCTVSGGSISSYYWSWIRQPPGRGLEWIGYIYYIGSTNYNPSLKSRVTISLDTSKNQFSLKLNSVTAADTAVYYCARDMMGALDYWGQGTLVTVSS

SEQ ID NO: 178 - protein, 111 aa, synthetic construct
  source 1..111: note = VL
DIVMTQTPLSSPVTLGQPASISCRSSESLVHSDGNTYLSWLQQRPGQPPRLLIYKISNRFSGVPDRFSGSGAGTDFTLKISRVEAEDVGVYYCMQAAQFPTFGQGTRLEIK

SEQ ID NO: 179-180 - skipped (2 entries)

SEQ ID NO: 181 - protein, 5 aa, synthetic construct
  source 1..5: note = VH-CDR1
SYYWS

SEQ ID NO: 182 - protein, 16 aa, synthetic construct
  source 1..16: note = VH-CDR2
RIYTSGSTNFKPSLKS

SEQ ID NO: 183 - protein, 9 aa, synthetic construct
  source 1..9: note = VH-CDR3
DPLGSFFDY

SEQ ID NO: 184 - protein, 14 aa, synthetic construct
  source 1..14: note = VL-CDR1
TGTSSDVGGYNYVS

SEQ ID NO: 185 - protein, 7 aa, synthetic construct
  source 1..7: note = VL-CDR2
EVSKRPS

SEQ ID NO: 186 - protein, 10 aa, synthetic construct
  source 1..10: note = VL-CDR3
SSYADINNLV

SEQ ID NO: 187 - protein, 117 aa, synthetic construct
  source 1..117: note = VH
QVQLQESGPGLVKPSETLSLTCTVSGGSISSYYWSWIRQPAGKGLEWIGRIYTSGSTNFKPSLKSRVTMSVDTSKNQFSLKLRSMTAADTAVYYCARDPLGSFFDYWGQGTLVTVSS

SEQ ID NO: 188 - protein, 110 aa, synthetic construct
  source 1..110: note = VL
QSALTQPPSASGSPGQSVTISCTGTSSDVGGYNYVSWYQQHPGKAPKLMIYEVSKRPSGVPDRFSGSKSGNTASLTVSGLQAEDEADYYCSSYADINNLVFGTGTKVTVL

SEQ ID NO: 189-190 - skipped (2 entries)

SEQ ID NO: 191 - protein, 5 aa, synthetic construct
  source 1..5: note = VH-CDR1
GYYMH

SEQ ID NO: 192 - protein, 17 aa, synthetic construct
  source 1..17: note = VH-CDR2
WINPNSGGTNYAQKFQG

SEQ ID NO: 193 - protein, 6 aa, synthetic construct
  source 1..6: note = VH-CDR3
GAWGSN

SEQ ID NO: 194 - protein, 11 aa, synthetic construct
  source 1..11: note = VL-CDR1
RASQGISNYLA

SEQ ID NO: 195 - protein, 7 aa, synthetic construct
  source 1..7: note = VL-CDR2
AASSLQS

SEQ ID NO: 196 - protein, 9 aa, synthetic construct
  source 1..9: note = VL-CDR3
QQYNSYPYT

SEQ ID NO: 197 - protein, 115 aa, synthetic construct
  source 1..115: note = VH
QVQLVQSGAEVKKPGASVKVSCKASGYTFTGYYMHWVRQAPGQGLEWMGWINPNSGGTNYAQKFQGRVTMTRDTSINTAYMELSRLRSDDTAVYYCARGAWGSNWGQGTLVTVSS

SEQ ID NO: 198 - protein, 107 aa, synthetic construct
  source 1..107: note = VL
DIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFGQGTKLEIK

SEQ ID NO: 199-200 - skipped (2 entries)

SEQ ID NO: 201 - protein, 5 aa, synthetic construct
  source 1..5: note = VH-CDR1
YFYWT

SEQ ID NO: 202 - protein, 16 aa, synthetic construct
  source 1..16: note = VH-CDR2
YIYHSGSTNYNPSLKS

SEQ ID NO: 203 - protein, 13 aa, synthetic construct
  source 1..13: note = VH-CDR3
DGGIVGATNALDI

SEQ ID NO: 204 - protein, 12 aa, synthetic construct
  source 1..12: note = VL-CDR1
RASQSVSSSYLA

SEQ ID NO: 205 - protein, 7 aa, synthetic construct
  source 1..7: note = VL-CDR2
GASSRAT

SEQ ID NO: 206 - protein, 9 aa, synthetic construct
  source 1..9: note = VL-CDR3
QQYGSSPLT

SEQ ID NO: 207 - protein, 121 aa, synthetic construct
  source 1..121: note = VH
QVQLQESGPGLVKPSETLSLTCTVSGGSIDYFYWTWIRQSPGKGLEWIGYIYHSGSTNYNPSLKSRVTMSLDTSKNQFSLKLTSVTTADTAVYYCARDGGIVGATNALDIWGQGTMVTVSS

SEQ ID NO: 208 - protein, 108 aa, synthetic construct; identical to SEQ ID NO: 128
  source 1..108: note = VL

SEQ ID NO: 209-210 - skipped (2 entries)

SEQ ID NO: 211 - protein, 5 aa, synthetic construct
  source 1..5: note = VH-CDR1
GYYWS

SEQ ID NO: 212 - protein, 16 aa, synthetic construct
  source 1..16: note = VH-CDR2
YIYYSVSSNYNPSLKS

SEQ ID NO: 213 - protein, 10 aa, synthetic construct
  source 1..10: note = VH-CDR3
ARDETGYFDY

SEQ ID NO: 214 - protein, 12 aa, synthetic construct
  source 1..12: note = VL-CDR1
RASQSVRSSYLA

SEQ ID NO: 215 - protein, 7 aa, synthetic construct
  source 1..7: note = VL-CDR2
GASSRAT

SEQ ID NO: 216 - protein, 8 aa, synthetic construct
  source 1..8: note = VL-CDR3
QQYGSSLT

SEQ ID NO: 217 - protein, 116 aa, synthetic construct
  source 1..116: note = VH
QVQLQESGPGLVKPSETLSLTCTVSGGSISGYYWSWIRQTPGKGLEWIGYIYYSVSSNYNPSLKSRVTISVDTSKNQFSLRLSSVTAADTAVYYCARDETGYFDYWGQGTLVTVSS

SEQ ID NO: 218 - protein, 107 aa, synthetic construct
  source 1..107: note = VL
EIVLTQSPGTLSLSPGERATLSCRASQSVRSSYLAWYQQKPGQAPRLLIYGASSRATGIPDRFSGSGSGTDFTLTISRLEPEDFAVYYCQQYGSSLTFGGGTKVEIK

SEQ ID NO: 219-220 - skipped (2 entries)

SEQ ID NO: 221 - protein, 5 aa, synthetic construct
  source 1..5: note = VH-CDR1
SYYWS

SEQ ID NO: 222 - protein, 16 aa, synthetic construct
  source 1..16: note = VH-CDR2
RLYTSGSTNYNPSLKS

SEQ ID NO: 223 - protein, 11 aa, synthetic construct
  source 1..11: note = VH-CDR3
ARDPLGSFFDY

SEQ ID NO: 224 - protein, 14 aa, synthetic construct
  source 1..14: note = VL-CDR1
TGTSSDVGGYNYVS

SEQ ID NO: 225 - protein, 7 aa, synthetic construct
  source 1..7: note = VL-CDR2
EVSKRPS

SEQ ID NO: 226 - protein, 10 aa, synthetic construct
  source 1..10: note = VL-CDR3
SSYAGSNNVV

SEQ ID NO: 227 - protein, 117 aa, synthetic construct
  source 1..117: note = VH
QVQLQESGPGLVKPSETLSLTCTVSGDSFSSYYWSWIRQPAGKGLEWIGRLYTSGSTNYNPSLKSRVTVSVDTSKNQFSLKLNSVTAADTAVYYCARDPLGSFFDYWGQGTLVTVSS

SEQ ID NO: 228 - protein, 110 aa, synthetic construct
  source 1..110: note = VL
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNVVFGGGTKLTVL

SEQ ID NO: 229-230 - skipped (2 entries)

SEQ ID NO: 231 - protein, 7 aa, synthetic construct
  source 1..7: note = VH-CDR1
TSGVGVG

SEQ ID NO: 232 - protein, 16 aa, synthetic construct
  source 1..16: note = VH-CDR2
LIYWDDDKLYSPSLKS

SEQ ID NO: 233 - protein, 9 aa, synthetic construct
  source 1..9: note = VH-CDR3
LGMRHAFDI

SEQ ID NO: 234 - protein, 14 aa, synthetic construct
  source 1..14: note = VL-CDR1
TGSSSNIGAGYDVH

SEQ ID NO: 235 - protein, 7 aa, synthetic construct
  source 1..7: note = VL-CDR2
GNRYRPS

SEQ ID NO: 236 - protein, 10 aa, synthetic construct
  source 1..10: note = VL-CDR3
QSYDSSLSVV

SEQ ID NO: 237 - protein, 119 aa, synthetic construct
  source 1..119: note = VH
QITLKESGPTLVKPTQTLTLTCTFSGFSLSTSGVGVGWIRQPPEKALEWLALIYWDDDKLYSPSLKSRLTITKDTSKNQVVLTLTNMDPVDTATYYCARLGMRHAFDIWGQGTMVTVSS

SEQ ID NO: 238 - protein, 110 aa, synthetic construct
  source 1..110: note = VL
QSVLTQPPSVSGAPGQRVTISCTGSSSNIGAGYDVHWFQQLPGTAPKLLIYGNRYRPSGVPDRFSGSKSGTSASLAIAGLQAEDEADYYCQSYDSSLSVVFGGGTKLTVL

SEQ ID NO: 239-240 - skipped (2 entries)

SEQ ID NO: 241 - protein, 5 aa, synthetic construct
  source 1..5: note = VH-CDR1
GYYWS

SEQ ID NO: 242 - protein, 16 aa, synthetic construct
  source 1..16: note = VH-CDR2
YVYYSVSTDYNPSLKS

SEQ ID NO: 243 - protein, 8 aa, synthetic construct
  source 1..8: note = VH-CDR3
DETGYFDY

SEQ ID NO: 244 - protein, 12 aa, synthetic construct
  source 1..12: note = VL-CDR1
RASQSVRSSYLA

SEQ ID NO: 245 - protein, 7 aa, synthetic construct
  source 1..7: note = VL-CDR2
GASSRAT

SEQ ID NO: 246 - protein, 8 aa, synthetic construct
  source 1..8: note = VL-CDR3
QQYGSSLT

SEQ ID NO: 247 - protein, 116 aa, synthetic construct
  source 1..116: note = VH
QVQLQESGPGLVKPSETLSLTCTVSGGSISGYYWSWIRQTPGKGLEWIGYVYYSVSTDYNPSLKSRVTISVDTSKNQFSLRLSSVTAADTAVYYCARDETGYFDYWGQGTLVTVSS

SEQ ID NO: 248 - protein, 107 aa, synthetic construct; identical to SEQ ID NO: 218
  source 1..107: note = VL

SEQ ID NO: 249-250 - skipped (2 entries)

SEQ ID NO: 251 - protein, 5 aa, synthetic construct
  source 1..5: note = VH-CDR1
TYYWS

SEQ ID NO: 252 - protein, 16 aa, synthetic construct
  source 1..16: note = VH-CDR2
RIYTSGTTNYNPSLKS

SEQ ID NO: 253 - protein, 9 aa, synthetic construct
  source 1..9: note = VH-CDR3
DPLGSFFDY

SEQ ID NO: 254 - protein, 14 aa, synthetic construct
  source 1..14: note = VL-CDR1
TGTSSDVGGYNYVS

SEQ ID NO: 255 - protein, 7 aa, synthetic construct
  source 1..7: note = VL-CDR2
EVSKRPS

SEQ ID NO: 256 - protein, 10 aa, synthetic construct
  source 1..10: note = VL-CDR3
SSYAGINNVV

SEQ ID NO: 257 - protein, 117 aa, synthetic construct
  source 1..117: note = VH
QVQLQESGPGLVKPSETLSLTCTVSGDSIRTYYWSWIRQPAGKGLEWIGRIYTSGTTNYNPSLKSRVTMSEDTSNNQFSLKLSSVTAADTAVYYCARDPLGSFFDYWGQGTLVTVSS

SEQ ID NO: 258 - protein, 110 aa, synthetic construct
  source 1..110: note = VL
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGINNVVFGGGTKLTVL

SEQ ID NO: 259-260 - skipped (2 entries)

SEQ ID NO: 261 - protein, 5 aa, synthetic construct
  source 1..5: note = VH-CDR1
SYDMH

SEQ ID NO: 262 - protein, 16 aa, synthetic construct
  source 1..16: note = VH-CDR2
AIGTAGDTYYPGSVKG

SEQ ID NO: 263 - protein, 11 aa, synthetic construct
  source 1..11: note = VH-CDR3
DSGGLSYAFDI

SEQ ID NO: 264 - protein, 11 aa, synthetic construct
  source 1..11: note = VL-CDR1
RASQGINNYLA

SEQ ID NO: 265 - protein, 7 aa, synthetic construct
  source 1..7: note = VL-CDR2
AASSLLS

SEQ ID NO: 266 - protein, 9 aa, synthetic construct
  source 1..9: note = VL-CDR3
LQHNSYPPT

SEQ ID NO: 267 - protein, 119 aa, synthetic construct
  source 1..119: note = VH
EVQLVESGGGLVQPGGSLRLSCAASGFTFSSYDMHWVRQATGKGLEWVSAIGTAGDTYYPGSVKGRFTISRENAKSSLYLQMNSLRAGDTAVYYCARDSGGLSYAFDIWGQGTMVTVSS

SEQ ID NO: 268 - protein, 107 aa, synthetic construct
  source 1..107: note = VL
DIQMTQSPSAMSASVGDRVAITCRASQGINNYLAWFQQKPGKVPKRLIYAASSLLSGVPSRFSGSGSGTEFTLTISSLQPEDFATYYCLQHNSYPPTFGQGTKVEIK

SEQ ID NO: 269-270 - skipped (2 entries)

SEQ ID NO: 271 - protein, 5 aa, synthetic construct
  source 1..5: note = VH-CDR1
SNYWT

SEQ ID NO: 272 - protein, 16 aa, synthetic construct
  source 1..16: note = VH-CDR2
YIYYIGSTNYNPSLKS

SEQ ID NO: 273 - protein, 15 aa, synthetic construct
  source 1..15: note = VH-CDR3
ARDGGYSGSYWGLDP

SEQ ID NO: 274 - protein, 12 aa, synthetic construct
  source 1..12: note = VL-CDR1
RASQSVSSRSLA

SEQ ID NO: 275 - protein, 7 aa, synthetic construct
  source 1..7: note = VL-CDR2
GASSRAT

SEQ ID NO: 276 - protein, 9 aa, synthetic construct
  source 1..9: note = VL-CDR3
QQYVTSPIT

SEQ ID NO: 277 - protein, 121 aa, synthetic construct
  source 1..121: note = VH
QVQLQESGPGLVKPSETLSLTCTVSGGSINSNYWTWIRQPPGKGLEWIGYIYYIGSTNYNPSLKSRVTISIDTSKNHFSLKLSSVTAADTAVYYCARDGGYSGSYWGLDPWGQGTLVTVSS

SEQ ID NO: 278 - protein, 108 aa, synthetic construct
  source 1..108: note = VL
EIVLTQSPGTLSLSPGERATLSCRASQSVSSRSLAWYQQKPGQAPRLLIYGASSRATGIPDRFSGSGSGTDFTLTISRLEPEDFAVYYCQQYVTSPITFGQGTRLEIK

SEQ ID NO: 279-280 - skipped (2 entries)

SEQ ID NO: 281 - protein, 5 aa, synthetic construct
  source 1..5: note = VH-CDR1
YYYWS

SEQ ID NO: 282 - protein, 16 aa, synthetic construct
  source 1..16: note = VH-CDR2
YIYFSGSTNYNPSLKS

SEQ ID NO: 283 - protein, 15 aa, synthetic construct
  source 1..15: note = VH-CDR3
ARDGGIVGATEAFDI

SEQ ID NO: 284 - protein, 12 aa, synthetic construct
  source 1..12: note = VL-CDR1
RASQSISGSYLA

SEQ ID NO: 285 - protein, 7 aa, synthetic construct
  source 1..7: note = VL-CDR2
GASSRAT

SEQ ID NO: 286 - protein, 9 aa, synthetic construct
  source 1..9: note = VL-CDR3
QQYGSSPIT

SEQ ID NO: 287 - protein, 121 aa, synthetic construct
  source 1..121: note = VH
QVQLQESGPGLVKPSETLSLTCTVSGGSISYYYWSWIRQPPGKGLEWIGYIYFSGSTNYNPSLKSRVTMSVDTSKNQFSLKLTSVTAADTAVYYCARDGGIVGATEAFDIWGQGTMVTVSS

SEQ ID NO: 288 - protein, 108 aa, synthetic construct
  source 1..108: note = VL
EIVLTQSPGTLSLSPGERATLSCRASQSISGSYLAWYQQKPGQAPRLLIYGASSRATGIPDRFSGSGSGTDFTLTISRLEPEDFAVYYCQQYGSSPITFGQGTRLEIK

SEQ ID NO: 289-290 - skipped (2 entries)

SEQ ID NO: 291 - protein, 5 aa, synthetic construct
  source 1..5: note = VH-CDR1
SYYWT

SEQ ID NO: 292 - protein, 16 aa, synthetic construct
  source 1..16: note = VH-CDR2
YIYYNGNTNYNPSLKS

SEQ ID NO: 293 - protein, 12 aa, synthetic construct
  source 1..12: note = VH-CDR3
ARDGGPWVPLDY

SEQ ID NO: 294 - protein, 12 aa, synthetic construct
  source 1..12: note = VL-CDR1
RASQSVSSSYLA

SEQ ID NO: 295 - protein, 7 aa, synthetic construct
  source 1..7: note = VL-CDR2
GASSRAT

SEQ ID NO: 296 - protein, 9 aa, synthetic construct
  source 1..9: note = VL-CDR3
QQYGSSPPT

SEQ ID NO: 297 - protein, 118 aa, synthetic construct
  source 1..118: note = VH
QVQLQESGPGLVKPSETLSLTCTVSGGSISSYYWTWIRQPPGKGLEWIGYIYYNGNTNYNPSLKSRVTMSVETSKNQFSLKLRSVTAADTAVYYCARDGGPWVPLDYWGQGTLVTVSS

SEQ ID NO: 298 - protein, 108 aa, synthetic construct
  source 1..108: note = VL
EIVLTQSPGTLSLSPGERATLSCRASQSVSSSYLAWYQQKPGQAPRLLIYGASSRATGIPDRFSGSGSGTDFTLTISRLEPEAFAVYYCQQYGSSPPTFGQGTKVEIK

SEQ ID NO: 299-300 - skipped (2 entries)

SEQ ID NO: 301 - protein, 5 aa, synthetic construct
  source 1..5: note = VH-CDR1
FYYWS

SEQ ID NO: 302 - protein, 16 aa, synthetic construct
  source 1..16: note = VH-CDR2
RIYTSGSTNYNPSLKS

SEQ ID NO: 303 - protein, 11 aa, synthetic construct
  source 1..11: note = VH-CDR3
ARDPLGTFFDY

SEQ ID NO: 304 - protein, 14 aa, synthetic construct
  source 1..14: note = VL-CDR1
TGTSSDVGGYNYVS

SEQ ID NO: 305 - protein, 7 aa, synthetic construct
  source 1..7: note = VL-CDR2
EVSKRPS

SEQ ID NO: 306 - protein, 10 aa, synthetic construct
  source 1..10: note = VL-CDR3
SSYAGINNVV

SEQ ID NO: 307 - protein, 117 aa, synthetic construct
  source 1..117: note = VH
QVQLQESGPGLVKPSETLSLTCTVSGSSISFYYWSWIRQPAGKGLEWIGRIYTSGSTNYNPSLKSRLTMSLDTSKNQFSLRLSSVTAADTAVYYCARDPLGTFFDYWGQGTLVTVSS

SEQ ID NO: 308 - protein, 110 aa, synthetic construct; identical to SEQ ID NO: 258
  source 1..110: note = VL

SEQ ID NO: 309-310 - skipped (2 entries)

SEQ ID NO: 311 - protein, 5 aa, synthetic construct
  source 1..5: note = VH-CDR1
SYYWS

SEQ ID NO: 312 - protein, 16 aa, synthetic construct
  source 1..16: note = VH-CDR2
RIYTIGTTNYNPSLKS

SEQ ID NO: 313 - protein, 9 aa, synthetic construct
  source 1..9: note = VH-CDR3
DPLGTFFDY

SEQ ID NO: 314 - protein, 14 aa, synthetic construct
  source 1..14: note = VL-CDR1
TGTSSDVGGHNYVS

SEQ ID NO: 315 - protein, 7 aa, synthetic construct
  source 1..7: note = VL-CDR2
EVTKRPS

SEQ ID NO: 316 - protein, 10 aa, synthetic construct
  source 1..10: note = VL-CDR3
SSYAGINNLV

SEQ ID NO: 317 - protein, 117 aa, synthetic construct
  source 1..117: note = VH
QVQLQESGPGLVKPSETLSLTCTVSGDSISSYYWSWIRQPAGKGLEWIGRIYTIGTTNYNPSLKSRVTMSVDTSKNQFSLKLNSVTAADAAVYYCARDPLGTFFDYWGQGFLVTVSS

SEQ ID NO: 318 - protein, 110 aa, synthetic construct
  source 1..110: note = VL
QSALTQPPSASGSPGQSVTISCTGTSSDVGGHNYVSWYQQHPGKAPKFMIYEVTKRPSGVPDRFSGSKSGNTASLTVSGLQAEDEADYYCSSYAGINNLVFGGGTKLTVL

SEQ ID NO: 319-320 - skipped (2 entries)

SEQ ID NO: 321 - protein, 5 aa, synthetic construct
  source 1..5: note = VH-CDR1
FYYWT

SEQ ID NO: 322 - protein, 16 aa, synthetic construct
  source 1..16: note = VH-CDR2
YIYHSGSTNYNPSLKS

SEQ ID NO: 323 - protein, 13 aa, synthetic construct
  source 1..13: note = VH-CDR3
DGGIVGATNAFDI

SEQ ID NO: 324 - protein, 12 aa, synthetic construct
  source 1..12: note = VL-CDR1
RASQSVTSSYLA

SEQ ID NO: 325 - protein, 7 aa, synthetic construct
  source 1..7: note = VL-CDR2
GASSRAT

SEQ ID NO: 326 - protein, 9 aa, synthetic construct
  source 1..9: note = VL-CDR3
QQYGSSPLT

SEQ ID NO: 327 - protein, 121 aa, synthetic construct
  source 1..121: note = VH
QVQLQESGPGLVKPSETLSLSCTVSGGSISFYYWTWIRQPPGKGLEWIGYIYHSGSTNYNPSLKSRVTISVDTSKNQFSLKLSSVTAADTAVYYCARDGGIVGATNAFDIWGQGTMVTVSS

SEQ ID NO: 328 - protein, 108 aa, synthetic construct
  source 1..108: note = VL
EIVLTQSPGTLSLSPGERATLSCRASQSVTSSYLAWYQQKPGQAPRLLIYGASSRATGIPDRFSGSGSGTDFTLTISRLEPEDFAVYYCQQYGSSPLTFGGGTKVEIK

SEQ ID NO: 329-330 - skipped (2 entries)

SEQ ID NO: 331 - protein, 5 aa, synthetic construct
  source 1..5: note = VH-CDR1
SYYWS

SEQ ID NO: 332 - protein, 16 aa, synthetic construct
  source 1..16: note = VH-CDR2
YIYYIGTTNYNPSLKS

SEQ ID NO: 333 - protein, 10 aa, synthetic construct
  source 1..10: note = VH-CDR3
DLGIEGGFDY

SEQ ID NO: 334 - protein, 11 aa, synthetic construct
  source 1..11: note = VL-CDR1
RASQSVSSSLA

SEQ ID NO: 335 - protein, 7 aa, synthetic construct
  source 1..7: note = VL-CDR2
GASTRAT

SEQ ID NO: 336 - protein, 9 aa, synthetic construct
  source 1..9: note = VL-CDR3
QQYNNWPLT

SEQ ID NO: 337 - protein, 118 aa, synthetic construct
  source 1..118: note = VH
QVQLQESGPGLVKPSETLSLTCTVSAVSINSYYWSWIRQPPGKGLEWIGYIYYIGTTNYNPSLKSRVTILLDTSKNQFSLKLSSVTAADTAVYYCARDLGIEGGFDYWGQGTLVTVSS

SEQ ID NO: 338 - protein, 107 aa, synthetic construct
  source 1..107: note = VL
EIVMTQSPASLSVSPGERATLSCRASQSVSSSLAWYQQKVGQAPRLLISGASTRATGIPARFSGSGSGTEFTLTISSLQSEDFAVYYCQQYNNWPLTFGGGTKVEIK

SEQ ID NO: 339-340 - skipped (2 entries)

SEQ ID NO: 341 - protein, 225 aa, Homo sapiens
  source 1..225: note = Human CD161
MDQQAIYAELNLPTDSGPESSSPSSLPRDVCQGSPWHQFALKLSCAGIILLVLVVTGLSVSVTSLIQKSSIEKCSVDIQQSRNKTTERPGLLNCPIYWQQLREKCLLFSHTVNPWNNSLADCSTKESSLLLIRDKDELIHTQNLIRDKAILFWIGLNFSLSEKNWKWINGSFLNSNDLEIRGDAKENSCISISQTSVYSEYCSTEIRWICQKELTPVRNKVYPDS